(19) United States
(12) Patent Application Publication
(10) Pub. No.: US 2025/0188183 A1
(43) Pub. Date: Jun. 12, 2025

(54) USE OF PROTOCADHERINS IN METHODS OF DIAGNOSING AND TREATING CANCER

(71) Applicant: Reverse Bioengineering, Inc., Alameda, CA (US)

(72) Inventor: Michael D. West, Mill Valley, CA (US)

(21) Appl. No.: 18/279,962

(22) PCT Filed: Mar. 2, 2022
(86) PCT No.: PCT/US2022/018556

§ 371 Date: Sep. 1, 2023

Related U.S. Application Data
(60) Provisional application No. 63/274,731, filed on Nov. 2, 2021.
(60) Provisional application No. 63/155,631, filed on Mar. 2, 2021.

(51) Int. Cl. C07K 16/30; A61K 39/00; A61P 35/00; C12Q 1/6886; G01N 33/574

(52) U.S. Cl. CPC C07K 16/3069; A61P 35/00; C12Q 1/6886; G01N 33/57492; A61K 2039/505; C12Q 2600/106; C12Q 2600/156

(57) ABSTRACT

Compositions and methods are disclosed for targeting the clustered protocadherins for therapeutic effect. More specifically, compositions and methods are disclosed to modify select isoforms in the α, β, and γ cluster to promote tissue regeneration or to target and reduce tumor burden in diverse cancer types. The methods have application in veterinary and human medicine.

### RELATED APPLICATIONS

The instant application claims priority to U.S. Provisional Application No. 63/155,631, filed Mar. 2, 2021; and U.S. Provisional Application No. 63/274,731, filed Nov. 2, 2021; entire contents of each of which are expressly incorporated by reference herein in their entireties.

### FIELD OF THE INVENTION

The present invention relates to compositions and methods for modulating of the activity of clustered protocadherin isoforms in cells within a mammal for therapeutic effect. More specifically, methods and formulations are described that alter the relative levels of said isoforms in mammalian cells to induce tissue regeneration or diagnose and treat cancer. Additionally, compositions and methods are disclosed to stimulate the body's immune response to said isoforms to generate an immunotherapeutic response to cancer.

### BACKGROUND

Advances in stem cell technology, such as the isolation and propagation in vitro of human pluripotent stem (hPS) cells constitute an important new area of medical research. hPS cells have a demonstrated potential to be propagated in the undifferentiated state and then to be induced subsequently to differentiate into any and all of the cell types in the human body, including complex tissues. This has led, for example, to the prediction that many diseases resulting from the dysfunction of cells may be amenable to treatment by the administration of human embryonic stem cell-derived of various differentiated types¹⁷.

In regard to differentiating hPS cells into desired cell types, the potential to clonally isolate lines of human embryonic progenitor cells provide a means to propagate novel highly purified cell lineages with a prenatal, more specifically, a pre-fetal (embryonic) pattern of gene expression useful for regenerating tissues such as skin in a scarless manner. Such cell types have important applications in research, and for the manufacture of cell-based therapies (see PCT application Ser. No. PCT/US2006/013519; U.S. patent application Ser. No. 11/604,047; and U.S. patent application Ser. No. 12/504,630, each of which is incorporated by reference herein in its entirety).

More recently, the potential of pluripotent stem cells and derived embryoid bodies for in vitro self-assembly into 3-dimensional organoids has generated interest as a potential pathway for both obtaining tissue for transplantation (Singh et al, Stem Cells Dev. 2015. 24(23): 2778-95) as well as modeling human embryonic development. In contrast to embryonic cells, fetal and adult-derived cells often show reduced potential for such organoid formation, organogenesis in vitro, and epimorphic regeneration in vivo. Epimorphic regeneration, sometimes referred to as “epimorphosis,” refers to a type of tissue regeneration wherein a blastema of relatively undifferentiated mesenchyme proliferates at the site of an injury followed by scarless regeneration of the original tissue histology.

The developmental timing of the loss of epimorphic potential cannot be fixed precisely, and likely varies with tissue type, nevertheless, the embryonic-fetal transition (EFT), or eight weeks of human development (Carnegie Stage 23; O'Rahilly, R., F. Müller (1987) Developmental Stages in Human Embryos, Including a Revision of Streeter's ‘Horizons’ and a Survey of the Carnegie Collection. Washington, Carnegie Institution of Washington) appears to temporally correspond to the loss of skin regeneration in placental mammals (Walmsley, G. G. et al 2015. Scarless Wound Healing: Chasing the Holy Grail Plast Reconstr Surg. 135(3):907-17). We previously disclosed that tissue regeneration, as opposed to scarring, reflects the presence of an embryonic as opposed to fetal or adult phenotype (see PCT Patent Application Ser. Nos. PCT/US2014/040601, PCT/US2017/036452, and PCT/US2020/025512, each of which is incorporated by reference herein in its entirety). Early research into the cellular basis of embryology suggested that the complexities of tissue morphogenesis must employ precise mechanisms for cell-cell recognition^{1, 2}. Such a program seems necessary for the generation of a uniformity of cell types, association with specific neighboring cells, and the formation of discrete boundaries. Evidence for such cell-cell recognition and adhesion began with the pioneering studies of H. V. Wilson who first reported the spontaneous reassociation and regeneration of sponges from dissociated cells³.

The evolution of more complex organisms carrying an increasingly broad array of differentiated cell types arguably would depend to an even greater extent on this phenomenon. Johannes (Hans) Holtfreter referred to this selective adhesion as “tissue affinity” and in 1939 theorized that, “ . . . attraction and repulsion phenomenon is operating between various cell types during development and that information on this system will yield valuable information concerning the shiftings and segregations of tissues during organogenesis.” As a result of such early studies, the elucidation of the role of differential cell-cell adhesion in morphogenetic process became widely recognized as a milestone in developmental biology comparable to the discovery of organizers by Spemann and Mangold⁴.

In the 1950s, experimental embryologists extended these studies to chick embryonic development. The regenerative nature of embryonic anlagen was widely utilized in experimental embryo transplant studies, such as the cross-transplantation of embryonic tissues from quail to chicken. Studies of disaggregated limb bud chondrogenic and myogenic cells as well as embryonic mesonephros and retina could re-associate and regenerate scarlessly, however, aggregation markedly decreased later in the course of development^{5,6}. This led to the question of the “why” and “how” of the developmental repression of cell-cell adhesion regulation during embryogenesis and the loss of regenerative potential.

In an attempt to answer the question as to “why” natural selection led to the repression of regeneration during development followed by aging, George Williams suggested the model of “antagonistic pleiotropy”, in part as an explanation of why “after a seemingly miraculous feat of morphogenesis a complex metazoan should be unable to perform the much simpler task of merely maintaining what is already formed⁷.” The theory posits that some traits selected for based on their value early in the reproductive period of the life cycle, have adverse effects in the post-reproductive years. One example is the nearly global repression of telomerase expression early in embryonic development and the re-expression of the gene (TERT) in ˜90% of cancer types⁸. The repression of TERT may reduce the risk of malignancy early in life, but may lead to cell senescence late in life.

The “how” of the regulation of differential embryonic cell adhesion was first envisioned by William Dreyer, William Gray, and Leroy Hood in 1967 in a largely theoretical paper on the molecular basis of immunoglobulin diversity. They proposed a complex family of cell adhesion molecules may have been co-opted during evolution to create diverse antibodies.⁹” In 1999, William Dreyer proposed that retrotransposition events evolved a complex family of “area code” cell adhesion molecules that potentially include immunoglobulins, protocadherins, and olfactory receptors that regulate the complexities of development¹⁰.

The characterization of the clustered protocadherin locus (CPL), revealed such a predicted complex family of contiguous genes that like the immunoglobulin loci, contains numerous variable exons that can combine with constant exons to create a vastly complex array of cell adhesion interfaces¹¹. The superfamily member genes are arranged in three clusters designated Pcdhα, Pcdhβ, and Pcdhγ. The α and γ clusters possess variable exons encoding cadherin ectodomains, one transmembrane, and short cytoplasmic domains. In contrast, the β cluster contains unique single exon genes. In Homo sapiens there are 15 α, 15 β, and 22 γ domains. Each variable domains is preceded by a promoter region. These proteins generate homophilic associations in trans as evidenced by the induction of cell aggregation when PCDH constructs are expressed in K562 cells^{12, 13} In neurons, a vast complexity can be generated by stochastic activation of isoforms in a biallelic fashion¹⁴. Cis-combinations of the PCDH γ genes alone have been estimated to generate a diversity of >10⁵ diversity in unique cell interfaces.

Following up on his hypothesis, William Dreyer proposed in 1999 that retrotransposition events evolved a complex family of adhesion molecules that regulate the development of the CNS, including the synapses in the olfactory bulb. Subsequently, the majority of research on CPL homophilic interactions has focused on neuronal self-recognition¹⁵ and circuit assembly¹². While the role of cadherins in cell adhesion and development is well-established¹⁶, the potential role of the CPL in morphogenesis and epimorphic regeneration outside of the CNS has not been elucidated.

The isolation of human embryonic stem cell lines provides an in vitro source of diverse human cell types including those capable of embryonic cell adhesion as evidenced by their spontaneous formation of embryoid bodies and organoids^{17,18}. Utilizing clonally-purified progenitor cell lines as a model the early regenerative phenotype of cells before the embryonic-fetal transition (EFT), we applied deep neural network algorithms to transcriptomic data from a relatively large numbers of embryonic vs adult cell and tissue samples and reported putative genes differentially expressed before and after the EFT¹⁹ (see PCT Application Ser. No. PCT/US2017/036452, incorporated herein by reference in its entirety). Among the pre-EFT genes was the cell adhesion gene, PCDHB2 (see id.).

We previously disclosed compositions and methods related to markers of the EFT in mammalian species and their use in modulating tissue regeneration (See, e.g. PCT Patent Application Ser. Nos. PCT/US2014/040601, PCT/US2017/036452, PCT/US2020/025512 and U.S. patent application Ser. No. 14/896,664, each of which is incorporated by reference herein in its entirety) the disclosures of which are hereby incorporated by reference in their entirety. Here we disclose the surprising differential expression of CPL isoforms in embryonic progenitors compared to adult normal and cancer cell lines and compositions and methods to modify CPL expression for therapeutic effect.

### SUMMARY

The present disclosure provides compounds, compositions, and methods useful for modifying the levels of select isoforms of the clustered protocadherin gene locus in mammalian cells. The compositions and methods utilized to modify said isoform expression are intended to alter the embryonic or fetal/adult phenotype of said cells and/or tissue to alter the natural potential of regeneration in cells and/or tissues, to modify aging in said cells and/or tissues, and to alter cancer cells by inducing the maturation of cancer cells (induced cancer maturation (iCM)) or reprogramming matured cancer cells back to an embryonic-like state to induce senolysis in cancer stem cells (iS-CSC). The compositions and methods of the present invention also are used for screening molecules for efficacy in modulating the embryonic or fetal/adult phenotype of cells, and for generating animal models for said research. The methods utilized in modifying gene expression in the present invention include methods of modifying regulatory noncoding RNAs and mRNAs involved in the embryonic-fetal transition (EFT) including gene therapy, RNA and miRNA-based therapy, and small molecule-based therapy.

According to one aspect, the present disclosure provides a method for inducing mammalian, tissue regeneration in a cell or tissue of a subject, comprised of the steps: 1) contacting the cell and/or tissue with an agent to induce a euchromatic state within one or more clustered protocadherin loci; and 2) contacting the cell and/or tissue with one or more factors selected from the group consisting of: OCT4, SOX2, KLF4, NANOG, ESRRB, NR5A2, CEBPA, MYC, LIN28A, LIN28B and TERT, wherein the one or more factors are capable of restoring an embryonic pattern of gene expression without inducing pluripotency.

In some embodiments, the agent is an histone H3K9 methyltransferase inhibitor. In some embodiments, the histone H3K9 methyltransferase inhibitor is SUV39H1, SUV39H2, SETDB1, or a combination thereof.

In some embodiments, the histone H3K9 methyltransferase inhibitor is siRNA against one or more histone H3K9 methyltransferases. In some embodiments, a vector comprises a sequence encoding the siRNA. In some embodiments, the vector is a plasmid. In some embodiments, the vector is a viral vector. In some embodiments, the viral vector is an adeno-associated viral vector.

In some embodiments, the subject is human.

In another aspect, the present disclosure provides a method for inducing mammalian, tissue regeneration in a cell or tissue of a subject, comprised of the steps: 1) contacting the cell and/or tissue with an agent to induce a euchromatic state within one or more clustered protocadherin loci using an inhibitor of histone H3K9 methyltransferases; and 2) contacting the cell and/or tissue with chemical iTR inducers, said iTR inducers selected from the group consisting of inhibitors of glycogen synthase 3 (GSK3), inhibitors of TGF-beta signaling, HDAC inhibitors, inhibitors of H3K4/9 histone demethylase LSD1, inhibitors of Dot1L, inhibitors of G9a, inhibitors of Ezh2, inhibitors of DNA methyltransferase, activators of 3′ phosphoinositide-dependent kinase 1, promoters of glycolysis, RAR agonists, agents that mimic hypoxia, activators of telomerase, inhibitors of the MAPK/ERK pathway, or combination thereof, thereby reverting fetal or adult-derived cells to their embryonic counterpart without reverting the cells in said tissue to pluripotent stem cells. In some embodiments, steps 1 and 2 are in vitro. In some embodiments, steps 1 and 2 are in vivo. In some embodiments, the iTR inducers include those capable in other conditions of improving the efficiency of inducing pluripotency in somatic cell types, that is, in generating iPS cells.

In some embodiments, the histone H3K9 methyltransferase inhibitor is SUV39H1, SUV39H2, SETDB1, or a combination thereof.

In some embodiments, the histone H3K9 methyltransferase inhibitor is siRNA against one or more histone H3K9 methyltransferases. In some embodiments, a vector comprises a sequence encoding the siRNA. In some embodiments, the vector is a plasmid. In some embodiments, the vector is a viral vector. In some embodiments, the viral vector is an adeno-associated viral vector.

In some embodiments, the subject is human.

In another aspect of the present disclosure, a method is disclosed for inducing mammalian tissue regeneration, comprised of the steps: 1) contacting the cell and/or tissue with an agent to induce a euchromatic state within one or more clustered protocadherin loci using an inhibitor of histone H3K9 methyltransferases; 2) contacting the cell and/or tissue with one or more nucleic acids encoding TERT; and contacting the cell and/or tissue with chemical iTR inducers, said iTR inducers selected from the group consisting of combinations of inhibitors of glycogen synthase 3 (GSK3), inhibitors of TGF-beta signaling, HDAC inhibitors, inhibitors of H3K4/9 histone demethylase LSD1, inhibitors of Dot1L, inhibitors of G9a, inhibitors of Ezh2, inhibitors of DNA methyltransferase, activators of 3′ phosphoinositide-dependent kinase 1, promoters of glycolysis, RAR agonists, agents that mimic hypoxia, activators of telomerase, inhibitors of the MAPK/ERK pathway, or combinations thereof, thereby reverting fetal or adult-derived cells to their embryonic counterpart without reverting the cells in said tissue to pluripotent stem cells. In some embodiments, steps 1 and 2 are in vitro. In some embodiments, steps 1 and 2 are in vivo. In some embodiments, the inducers include those capable in other conditions of improving the efficiency of inducing pluripotency in somatic cell types, that is, in generating iPS cells.

In some embodiments, the subject is human.

In another aspect, the present disclosure provide for a method for treating cancer in a subject, a method comprising steps: 1) obtaining a biological sample from the subject comprising cancer cells; 2) identifying cancer cells that exhibit an embryonic phenotype; 3) identifying one or more isoforms of a clustered protocadherin locus expressed in the cancer cell that exhibit an embryonic phenotype; and 4) administering to the subject an anti-cancer vaccine comprising an antigen, thereby inducing an immune response.

In some embodiments, the identified one or more isoforms are members of the alpha cluster protocadherins and/or beta cluster protocadherins.

In some embodiments, the one or more isoforms are PCDHA1, PCDHA3, PCDHA6, PCDHB3, or a combination thereof.

In some embodiments, the antigen is one or more isoforms of the alpha cluster protocadherins and/or beta cluster protocadherins.

In some embodiments, the anti-cancer vaccine is mRNA.

In some embodiments, the mRNA encodes one or more isoforms of the alpha cluster protocadherins and/or beta cluster protocadherins.

In some embodiments, the mRNA encodes PCDHA1.

In some embodiments, the mRNA encodes PCDHA3.

In some embodiments, the mRNA encodes PCDHA6.

In some embodiments, the mRNA encodes PCDHB3.

In some embodiments, the anti-cancer vaccine is one or more polypeptides of isoforms of the alpha cluster protocadherins and/or beta cluster protocadherins, or fragments thereof.

In some embodiments, the one or more polypeptides are PCDHA1, PCDHA3, PCDHA6, PCDHB3, fragments thereof, or a combination thereof.

In some embodiments, the one or more polypeptides are PCDHA1, or fragments thereof.

In some embodiments, the one or more polypeptides are PCDHA3, or fragments thereof.

In some embodiments, the one or more polypeptides are PCDHA6, or fragments thereof.

In some embodiments, the one or more polypeptides are PCDHB3, or fragments thereof.

In some embodiments, a vector comprises the mRNA.

In some embodiments, the vector is a plasmid.

In some embodiments, the vector is a viral vector.

In some embodiments, the viral vector is an adeno-associated viral vector.

In some embodiments, a lipid formulation comprises the anti-cancer vaccine.

In another aspect, the present disclosure provide for a method is disclosed for inducing an immune response against mammalian cancer cells in a subject, comprised of the steps: 1) obtaining a biological sample from the subject comprising cancer cells; 2) identifying cancer cells that exhibit an embryonic phenotype; 3) identifying one or more isoforms of a clustered protocadherin locus expressed in the cancer cell that exhibit an embryonic phenotype; and 4) administering to the subject genetically-modified immune cells capable of generating an immune response to the cancer in the subject. In some embodiments, the one or more isoforms are PCDHA1, PCDHA3, PCDHA6, PCDHB3, or a combination thereof.

In some embodiments, the genetically-modified immune cells are derived from pluripotent stem cells. In some embodiments, the pluripotent stem cells are derived from cells from the subject. In some embodiments, the pluripotent stem cells are derived from cells from a donor.

In some embodiments, the genetically-modified immune cells are Chimeric Antigen Receptor T-cells (CAR T-cells). In some embodiments, the CAR comprises an antigen binding domain that binds an isoform of the clustered protocadherin locus expressed in the cancer cell. In some embodiments, the isoform is a member of the alpha cluster protocadherins and/or beta cluster protocadherins.

In some embodiments, the isoform are PCDHA1, PCDHA3, PCDHA6, PCDHB3, fragment thereof, or a combination thereof.

In some embodiments, the CAR T-cells are derived from pluripotent stem cells. In some embodiments, the pluripotent stem cells are derived from cells from the subject. In some embodiments, the pluripotent stem cells are derived from cells from a donor.

In another aspect, the present disclosure provide for a method is disclosed for inducing an immune response against mammalian cancer cells in a subject, comprised of the steps: 1) obtaining a biological sample from the subject comprising cancer cells; 2) identifying cancer cells that exhibit an embryonic phenotype; 3) identifying one or more isoforms of a clustered protocadherin locus expressed in the cancer cell that exhibit an embryonic phenotype; and 4) administering to the subject an immunoglobulin superfamily member to direct an immune response specifically to the cancer cells. In some embodiments, the one or more isoforms are PCDHA1, PCDHA3, PCDHA6, PCDHB3, or a combination thereof.

In some embodiments., the immunoglobulin superfamily member is a monoclonal or polyclonal antibody. In some embodiments, the antibody binds PCDHA3. In some embodiments, the antibody binds PCDHB3.

In another aspect, the present disclosure provide for a method is disclosed for inducing an immune response against mammalian cancer cells in a subject, comprised of the steps: 1) obtaining a biological sample from the subject comprising cancer cells; 2) identifying cancer cells that exhibit an embryonic phenotype; 3) identifying one or more isoforms of a clustered protocadherin locus expressed in the cancer cell that exhibit an embryonic phenotype; and 4) administering to the subject a T-cell activating bispecific antigen-binding molecule wherein first antigen-binding moiety binds one or more polypeptides, or fragments thereof, encoded by the alpha and/or beta clustered protocadherin loci and the second antigen-binding molecule binds CD3, thereby activating T-cells.

In some embodiments, the one or more isoforms are encoded by the alpha and/or beta clustered protocadherin loci. In some embodiments, the one or more isoforms are PCDHA1, PCDHA3, PCDHA6, PCDHB3, or a combination thereof.

In another aspect, the present disclosure provide for a method is disclosed for inducing an immune response against mammalian cancer cells in a subject, comprised of the steps: 1) obtaining a biological sample from the subject comprising cancer cells; 2) identifying cancer cells that exhibit an embryonic phenotype; 3) identifying one or more isoforms of a clustered protocadherin locus expressed in the cancer cell that exhibit an embryonic phenotype; and 4) administering to the subject genetically-modified dendritic cells presenting one or more isoforms of a clustered protocadherin locus expressed in the cancer cell, thereby inducing an immune response to the cancer in the subject.

In some embodiments, the one or more isoforms of a clustered protocadherin locus expressed in the cancer cell are isoforms from the alpha and/or beta clustered protocadherin loci. In some embodiments, the one or more isoforms are PCDHA1, PCDHA3, PCDHA6, PCDHB3, or a combination thereof.

In some embodiments, the genetically-modified dendritic cells are derived from pluripotent stem cells. In some embodiments, the pluripotent stem cells are derived from cells from the subject. In some embodiments, the pluripotent stem cells are derived from cells from a donor.

In another aspect, the present disclosure provide for a method for treating cancer in a subject, a method comprising steps: 1) obtaining a biological sample from the subject comprising cancer cells; 2) identifying cancer cells that exhibit an embryonic phenotype; 3) identifying one or more isoforms of a clustered protocadherin locus expressed in the cancer cell that exhibit an embryonic phenotype; and 4) administering to the subject peptide sequences from said isoforms of the clustered protocadherin locus, thereby competitively interfering with cancer cell-cell adhesion in the subject.

In some embodiments, the peptide sequences are from polypeptides, or fragments thereof, encoded by the alpha and/or beta clustered protocadherin loci. In some embodiments, the one or more isoforms are PCDHA1, PCDHA3, PCDHA6, PCDHB3, or a combination thereof.

In another aspect, the present disclosure provide for a method for treating cancer in a subject, a method comprising steps: 1) obtaining a biological sample from the subject comprising cancer cells; 2) identifying cancer cells that exhibit an embryonic phenotype; 3) identifying one or more isoforms of a clustered protocadherin locus expressed in the cancer cell that exhibit an embryonic phenotype; and 4) administering to the subject small molecules that interfere with homologous interactions of said isoforms of the clustered protocadherin locus, thereby competitively interfering with cancer cell-cell adhesion in the subject.

In some embodiments, the method further comprises administering a chemotherapeutic agent to the subject.

In some embodiments, the chemotherapeutic agent is a DNA damaging agent, checkpoint inhibitor, antibody, alkylating agent, antimetabolites, anthracyclines, nitrosoureas, topisomerase inhibitor, isomerase inhibitor, mitotic inhibitor, tyrosine kinase inhibitors, protease inhibitor, or a combination thereof.

In some embodiments, the DNA damaging agent is high dose platinum-based alkylating chemotherapy, platinum compounds, thiotepa, cyclophosphamide, iphosphamide, nitrosureas, nitrogen mustard derivatives, mitomycins, epipodophyllotoxins, camptothecins, anthracyclines, poly(ADP-ribose) polymerase (PARP) inhibitors, ionizing radiation, ABT-888, olaparib (AZT-2281), gemcitabine, CEP-9722, AG014699, AG014699 with Temozolomide, BSI-201, or a combination thereof.

In some embodiments, the cancer cells that exhibit an embryonic phenotype express one or more of SOX2, KLF4, OCT4, MYC, NANOG, LIN28A, LIN28B, ESRRB, NR5A2, TERT, SSEA, TRA, and CEBPA. In some embodiments, the cancer cells that exhibit an embryonic phenotype expresses low level or no COX7A1.

In some embodiments, the one or more isoforms of protocadherin cluster proteins expressed in the cancer cell that exhibits an embryonic phenotype is PCDHA2, PCDHA4, PCDHA10, PCDHA12, PCDHB2, PCDHB5, PCDHB9, PCDHB10, PCDHB13, PCDHB14, PCDHB16, PCDHGB4, PCDHGB6, or a combination thereof.

In some embodiments, the subject is human.

In some embodiments, the cancer is B cell cancer, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, bone cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, polycythemia vera, lymphoma, multiple myeloma, bladder cancer, colon cancer, gynecologic cancers, renal cancer, laryngeal cancer, oral cancer, head and neck cancer, or skin cancer. In other embodiments, the cancer is breast cancer, prostate cancer, lung cancer, or colon cancer.

In some embodiments, the biological sample is from breast cancer or lung cancer.

Numerous aspects of aging and age-related disease are taught in the present invention to be addressable by modifying the expression of the isoforms of the clustered protocadherin locus disclosed herein. This breadth of application reflects the pan-tissue alteration in expression associated with the loss of regeneration during development and oncogenesis. These manifestations of aging include age-related vascular dysfunction including peripheral vascular, coronary, and cerebrovascular disease; musculoskeletal disorders including osteoarthritis, intervertebral disc degeneration, bone fractures, tendon and ligament tears, and limb regeneration; neurological disorders including stroke and spinal cord injuries; muscular disorders including muscular dystrophy, sarcopenia, myocardial infarction, and heart failure; endocrine disorders including Type I diabetes, Addison's disease, hypothyroidism, and pituitary insufficiency; digestive disorders including pancreatic exocrine insufficiency; ocular disorders including macular degeneration, retinitis pigmentosa, and neural retinal degeneration disorders; dermatological conditions including skin burns, lacerations, surgical incisions, alopecia, graying of hair, and skin aging; pulmonary disorders including emphysema and interstitial fibrosis of the lung; and auditory disorders including hearing loss.

In another aspect, the disclosure provides methods of modifying the expression of the isoforms of the clustered protocadherin locus in microbiopsies ex vivo to restore them to a state wherein they are capable of regenerating tissue scarlessly when transplanted.

In another aspect, the disclosure provides methods of modifying the expression of the isoforms of the clustered protocadherin locus in vivo to restore them to a state wherein they are capable of participating in Induced Tissue Regeneration (iTR).

In another aspect of the invention, methods are provided to cause iTR in tissues afflicted with degenerative disease including, but not limited to age-related disease wherein the means of effecting iTR in the diseased tissue utilizes a gene expression vector or vectors that cause the exogenous expression of the isoforms of the clustered protocadherin locus disclosed herein including but not limited to a member of the α and β cluster together with telomerase catalytic component, such as human TERT.

In another aspect of the invention, methods are provided to cause iTR in tissues afflicted with degenerative disease including, but not limited to age-related disease and cancer wherein the means of effecting iTR in the diseased tissue utilizes a gene expression vector or vectors that cause the exogenous expression of the isoforms of the clustered protocadherin locus disclosed herein including but not limited to a member of the α and β cluster.

In another aspect of the invention, methods are provided to cause iTR in tissues afflicted with degenerative disease including, but not limited to age-related disease and cancer wherein the means of effecting iTR in the diseased tissue utilizes a gene expression vector or vectors that inhibit the expression of the isoforms of the clustered protocadherin locus disclosed herein including but not limited to a member of the Q cluster.

In another aspect of the invention, methods are provided to cause iTR in tissues afflicted with degenerative disease including, but not limited to age-related disease and cancer wherein the means of effecting iTR in the diseased tissue utilizes a gene expression vector or vectors that inhibit the expression of the isoforms of the clustered protocadherin locus disclosed herein including but not limited to PCDHGA12.

In another aspect, the disclosure provides a method of identifying a candidate modulator of clustered protocadherin isoform activity comprising: (a) the candidate modulator or multiplicity of modulators of said isoform activity in a purified state or in a mixture with other molecules; (b) somatic cells exhibiting a fetal or adult pattern of gene expression as opposed to an embryonic pattern of gene expression; (c) a reporter construct present within the somatic cells or within extracts of said cells incapable of otherwise expressing embryonic isoforms of interest wherein the promoter of a gene differentially regulated in somatic cells in the embryonic phases of development compared to fetal and adult stages drives the expression of a reporter gene; and (ii) determining whether the candidate modulator or a multiplicity of modulators affect expression of the reporter gene, wherein altered expression of the reporter gene as compared with expression of the gene in the absence of the candidate modulator indicates that the compound modulates the isoform activity to resemble embryonic expression.

In another aspect, the disclosure provides a method of identifying a candidate modulator of clustered protocadherin isoform activity comprising: (a) the candidate modulator or multiplicity of modulators of said isoform activity in a purified state or in a mixture with other molecules; (b) somatic cells exhibiting an embryonic pattern of gene expression of a clustered protocadherin gene isoform of interest as opposed to an adult pattern of gene expression; (c) a reporter construct present within the somatic cells or within extracts of said cells incapable of otherwise expressing adult isoforms of interest wherein the promoter of a gene differentially regulated in somatic cells in the embryonic phases of development compared to fetal and adult stages drives the expression of a reporter gene; and (ii) determining whether the candidate modulator or a multiplicity of modulators affect expression of the reporter gene, wherein altered expression of the reporter gene as compared with expression of the gene in the absence of the candidate modulator indicates that the compound modulates the isoform activity to resemble adult expression.

In some embodiments, a method of identifying a candidate modulator of clustered protocadherin isoform expression further comprises administering a candidate compound or multiplicity of compounds identified as modulators of clustered protocadherin isoform expression to a subject.

In some embodiments, a method of identifying a candidate global modulator of clustered protocadherin isoform expression further comprises administering a candidate compound for induced tissue regeneration to cells derived from fetal or adult sources and assaying the expression clustered protocadherin isoform from the α or β cluster expression through the use of an easily measured readout such as fluorescence generated from GFP driven by the promoter of said isoform.

In some embodiments, a method of identifying a candidate modulator of clustered protocadherin isoform expression further comprises administering a candidate compound for induced tissue regeneration to cells derived from fetal or adult sources and assaying the expression of α or β cluster expression through the assay of the degree of methylation of the CpG island associated with said isoform.

In some embodiments, a method of identifying a compound further comprises administering the compound to a subject. In some embodiments, the subject is a non-human animal, e.g., a non-human animal that serves as a model for tissue regeneration, wound healing, or cancer. In some embodiments, the subject is a human.

In another aspect, the present disclosure provides a pharmaceutical composition comprising: (a) a modulator of clustered protocadherin isoform expression; and (b) a pharmaceutically acceptable carrier.

In another aspect, genes regulating clustered protocadherin isoform expression are altered such that cancer cells are treated with agents that alter the expression of the genes from that of an embryonic state to that of a fetal or adult state to cause induced cancer maturation (iCM).

In another aspect, genes regulating clustered protocadherin isoform expression are altered such that cancer cells are treated with agents that alter the expression of the genes from that of a fetal or adult state to that of an embryonic state to cause Induced Senolysis of Cancer Stem Cells (iS-CSC).

Certain conventional techniques of cell biology, cell culture, molecular biology, microbiology, recombinant nucleic acid (e.g., DNA) technology, immunology, etc., which are within the skill of the art, may be of use in aspects of the invention. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., editions as of 2008; Sambrook, Russell, and Sambrook, Molecular Cloning: A Laboratory Manual, 3.sup.rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Burns, R., Immunochemical Protocols (Methods in Molecular Biology) Humana Press; 3rd ed., 2005, Monoclonal antibodies: a practical approach (P. Shepherd and C Dean, eds., Oxford University Press, 2000); Freshney, R. I., “Culture of Animal Cells, A Manual of Basic Technique”, 5th ed., John Wiley & Sons, Hoboken, N J, 2005). All patents, patent applications, websites, databases, scientific articles, and other publications mentioned herein are incorporated herein by reference in their entirety.

In another aspect, the present invention provides a means of engineering an animal model, preferably a mouse model capable of robust regenerative potential, said mouse being in a common laboratory strain of mice thereby facilitating molecular genetics and animal preclinical studies. Said robustly regenerating mouse is produced by creating mice that express either inducibly in all tissues or select tissues, or constitutively expressing various combinations of isoforms from the α, β, and γ clusters wherein said mice and breeding said mice together, provide for mouse models of regeneration, aging, and cancer.

In another aspect, the present invention provides a method of identifying cancer cells in a subject, a method comprising a) providing a sample from a subject, wherein the sample comprises cells; b) determining one or more of the cells in a sample exhibit an embryonic phenotype; and c) identifying one or more isoforms of a clustered protocadherin locus expressed in the one or more of the cells in a sample exhibit an embryonic phenotype, wherein the one or more cells that exhibit embryonic phenotype and express one or more isoforms of a clustered protocadherin locus are cancer cells.

In some embodiments, the method provides further administering to the subject a) nucleotides encoding the one or more isoforms of a clustered protocadherin locus or a polypeptide, or a fragment thereof, of the one or more isoforms of a clustered protocadherin locus; or b) a genetically-modified immune cell capable of generating a immune response to the cancer in the subject.

### BRIEF DESCRIPTION OF THE DRAWINGS

The patent or application file contains at least one drawing executed in color. Copies of this patent or patent application publication with color drawing(s) will be provided to the Office upon request and payment of the necessary fee.

FIG. 1 depicts a volcano plot of differentially-expressed transcript isoforms determined by RNA-seq. Log₂ fold-change (FC) versus −log₁₀ (Bonferroni-adjusted p-value) is plotted. The underlying data is derived from RNA sequencing FPKM values for 42 diverse human clonal embryonic progenitor cell lines and 92 diverse adult-derived stromal and parenchymal cell types. The horizontal dotted line indicates a linear x-adjusted p-value of 0.05 and vertical dotted lines indicate a linear fold-change (FC) of 2. Transcripts passing the cutoff criteria of p<0.05 and FC>2 (elevated expression in adult) are represented with red points; transcripts with p<0.05 and FC<−2 (elevated expression in embryonic) are represented with green points. Points labelled in blue are CPL isoforms.

FIG. 2A depicts differential expression of CPL genes in embryonic vs adult cell types. Mean expression in FPKM of RNA-seq reads of PCDHA2, PCDHA4, PCDHA10, and PCDHA12 in diverse pluripotent stem cell (PC), hES-derived clonal embryonic progenitors (EP), fetal-derived cells (FC), adult-derived cells (AC), and neuronal cell (NC) types.

FIG. 2B depicts the mean expression of PCDHB2, PCDHB2, PCDHB2, PCDHB2, PCDHB2, and PCDHB2 in embryonic vs adult cell types.

FIG. 2C depicts mean expression of PCDHGB4, PCDHGB5, PCDHGB6, and PCDHGA12 in diverse embryonic, adult, and cancer cell types. ES and iPS cell lines include four different human ES cell lines and two iPS cell lines; Diverse EPs include 42 diverse human clonal embryonic progenitor cell lines, fetal-derived cells include three cultured of brown preadipocytes and five fetal arm skin fibroblast cultures from 8-16 weeks of gestation; Diverse Normal Somatic Cells include 87 diverse stromal and parenchymal cell types; five neuronal cell cultures including neurons and diverse astrocyte cell types; Epithelial cells include 25 diverse human epithelial cell types; Sarcomas include 39 diverse human sarcoma cell lines; Carcinomas include 33 diverse carcinoma and adenocarcinoma cell lines; (See Supplementary Table I for complete cell type descriptions.) (ns—not significant) (*p<0.05) (**p<0.01) (***p<0.001) (****p<0.0001). Error bars S.E.M.

FIG. 3 depicts differential expression of PCDHA4, PCDHB2, and PCDHGA12 in diverse embryonic, adult, and cancer cell types. Mean expression in FPKM of RNA-seq reads from pluripotent stem cell lines include four different human ES cell lines and two iPS cell lines (PC); 42 diverse hES-derived clonal embryonic progenitors (EP); Adult non-epithelial (ANE) cells include 97 diverse stromal and parenchymal cell types; Adult epithelial cells (AEC) include 25 diverse human epithelial cell types; Sarcoma cell (SC) lines include 39 diverse sarcoma cell types; Carcinomas and adenocarcinoms (CAC) include 33 diverse carcinoma and adenocarcinoma cell lines. (See Supplementary Table III for complete cell type descriptions.) (ns—not significant) (*p<0.05) (**p<0.01) (***p<0.001) (****p<0.0001). Error bars S.E.M.

FIGS. 4A-4C depicts correlations of embryonic and adult markers in sarcoma cell lines. FPKM values of the embryonic markers FIG. 4A) PCDHA4, FIG. 4B) PCDHB2 and FIG. 4C) PCDHGA12 are plotted against the adult marker COX7A1 in 39 diverse sarcoma cell lines.

FIG. 5 depicts IGV view of chromosomal features and RNA-sequence reads from the CPL. Rows represent: 1) ATAC-seq of the embryonic progenitor osteogenic mesenchymal line 4D20.8 (Embr Mesen); the embryonic vascular endothelial line 30-MV2-6 (Embr Endo); adult-derived osteogenic mesenchyme (MSCs); and adult-derived human aortic endothelial cells (HAECs). Blue asterisks make region of apparent decreased accessibility in adult cells; 2) CpG islands (CpGIs) downloaded from UCSC (cpgIslandExt.hg38.bed) where length>200 bp and >60% of expected CpGs based on G and C content; 3) CTCF binding sites in Embr Mesen, Embry Endo, Adult Mesen, and Adult Endo based on TOBIAS analysis of ATAC footprints (TOB-CTCF); 4) Differentially Methylated Regions (DMRs) are shown where elevated green represents hypermethylation in embryonic cells and depressed red signal represents relative hypomethylation in embryonic compared to adult cells; and 5) RNA sequence reads for the alpha, beta, and gamma loci. (hg38 Chromosome position: Chr5: 140,650,000-141,700,000), image captured from IGV.

FIG. 6 depicts percent methylation of representative significant DMRs within the CPL. Percent methylated CpGs is compiled for representative DMRs of the (α, β, and γ clusters (PCDHA4, PCDHB2, and PCDHGA12 respectively) for embryonic progenitor cells (EPs), Adult non-epithelial stromal and parenchymal cell types (ANE), and sarcoma cell lines (SC). Error bars S.E.M.

FIG. 7 depicts chromatin architecture in the CPL. Rows represent: 1) chromatin cis-interactions determined by Hi-C analysis showing associations between the location of superenhancers in the CPL region with the promoters of the gamma locus of isoforms in adult cells (arrows); 2) ATAC-seq of the embryonic progenitor osteogenic mesenchymal line 4D20.8 (Embr Mesen) and adult-derived osteogenic mesenchyme (MSCs). Blue asterisks make region of apparent decreased accessibility in adult cells; 3) Differentially Methylated Regions (DMRs) are shown where elevated green represents hypermethylation in embryonic cells and depressed red signal represents relative hypomethylation in embryonic compared to adult cells; 4) CTCF binding sites in Embr Mesen and Adult Mesen based on TOBIAS analysis of ATAC footprints (TOB-CTCF); 5) ChIP-seq values for CTCF; ChIP-seq results for LMNB1, LMNA (micrococcal nuclease treated (MNase), LMNA (sonicated); 6) location of superenhancers in the CPL region. 7) ChIP-seq data in paired embryonic and adult cells resulting from precipitation of chromatin with antibodies directed to H3K27me3, H3K27Ac, H2K4me3, H2K9me3, and H4K20me3, and H3K9Ac for the entire CPL. (hg38 Chromosome position: Chr5: 140,650,000-141,700,000), image captured from IGV.

FIG. 8 depicts heat map of expression of all CPL genes in human ES cells, diverse embryonic progenitor cell types, and adult cells. RNA-seq FPKM values for isoform expression of every isoform of the α, β, and γ clusters in the CPL is heat mapped for select pluripotent stem cells (human ES cells), EP cell lines and adult cell types.

FIG. 9 depicts PCDHGB4 and PCDHGA12 gene expression during aging in vitro and in HGPS. RNA-seq FPKM values for PCDHGB4 (blue) and PCDHGA12 (red) isoform expression in synchronized diverse dermal adult fibroblasts (aged 11-83 years); young (P19) and senescent (P38) GM00498 (3 year-old door) dermal fibroblasts, dermal fibroblasts from Hutchinson-Gilford progeria syndrome (HGPS) patients and age-matched controls. (*** p<0.001) (****p<0.0001) Error bars represent mean and standard error of the mean.

FIG. 10 depicts a model of potential transition in chromatin architecture during development, cellular aging, and cancer. During embryogenesis (up to approximately Carnegie Stage 23 of mammalian development), Lamin B1 predominates and facilitates CTCF binding, topological domains, and expression of cell type-specific CPL isoforms from the α and β clusters. Once organogenesis is complete, up-regulation of Lamin A facilitates an alteration in topological domains such that only isoforms from the γ cluster are expressed. In replicative senescence, LMNB1 expression is down-regulated together with expression of genes in the γ cluster. The majority of cancer cell lines show a CpG Island Methylator Phenotype (CIMP) in the CPL locus designated herein as CIMP-E.

FIG. 11 shows cell counts of the breast cancer cell line BT-20 which expresses the CPL isoform PCDHB3 following treatment with isotype antibody control, or polyclonal antibody directed to the beta cluster CPL isoform PCDHB3 or a combination of polyclonal antibody to PCDHA3, A6, and B3 together with PBMCs. (* indicated statistical significance p<0.05)

FIG. 12 shows cell counts of the lung cancer cell line NCI-H358 which expresses the CPL isoform PCDHA3 following treatment with isotype antibody control, or polyclonal antibody directed to the beta cluster CPL isoform PCDHA3 or a combination of polyclonal antibody to PCDHA1, A3, and B6 together with PBMCs. (* indicated statistical significance p<0.05)

FIG. 13 shows cell counts of the normal human dermal fibroblast cell strain MDW-1 which does not express alpha or beta CPL isoforms following treatment with isotype antibody control, or polyclonal antibody directed to the beta cluster CPL isoform PCDHA3, PCDHB3, a combination of polyclonal antibody to PCDHA1, A3, and B6, a combination of polyclonal antibody to PCDHA3, A6, and B3 together with PBMCs.

### DETAILED DESCRIPTION

### Abbreviations

Ab—Antibody
AC—Adult-derived cells
AEC—Adult epithelial cells
AMH—Anti-Mullerian Hormone
ANE—Adult non-epithelial cells
ASC—Adult stem cells
ATAC-seq—Transposase-Accessible Chromatin followed by sequencing
CAC—Carcinoma and adenocarcinoma cells
CAR T-Cells—Chimeric antigen receptor modified T-cells
cGMP—Current Good Manufacturing Processes
CIMP—CpG Island Methylator Phenotype
CIMP-E—CpG Island Methylator Phenotype refers to the unique DMRs of hypermethylated sites in embryonic cells compared to their fetal and adult counterparts
CM—Cancer Maturation
CNS—Central Nervous System
CpG—CpG dinucleotide
CPL—Clustered protocadherin locus which includes all isoforms encoded by the alpha, beta, and gamma loci
DMEM—Dulbecco's modified Eagle's medium
DMR—Differentially-methylated region
DMSO—Dimethyl sulphoxide
DNAm—Changes in the methylation of DNA that provide a marker or “clock” of the age of cells and tissue.
DPBS—Dulbecco's Phosphate Buffered Saline
ED Cells—Embryo-derived cells; hED cells are human ED cells
EDTA—Ethylenediamine tetraacetic acid
EFT—Embryonic-Fetal Transition
EPs—Embryonic progenitor cells
ES Cells—Embryonic stem cells; hES cells are human ES cells
ESC—Embryonic Stem Cells
EVs—Extracellular Vesicles
FACS—Fluorescence activated cell sorting
FBS—Fetal bovine serum
FCs—Fetal cells
FPKM—Fragments Per Kilobase of transcript per Million mapped reads from RNA sequencing.
GFP—Green fluorescent protein
GMP—Good Manufacturing Practices
HAEC—Human Aortic Endothelial Cell
hEC Cells—Human Embryonal Carcinoma Cells
hED Cells—Human embryo-derived cells
hEG Cells—“Human embryonic germ cells” are stem cells derived from the primordial germ cells of fetal tissue.
hEP cells—human embryonic progenitor cells
hES cells—human Embryonic Stem Cells including human ES-like cells, therefore “hES cells or “hESCs) as used herein refer to both primed and naïve pluripotent stem cells.
hiPS Cells—“Human induced pluripotent stem cells” are cells with properties similar to hES cells obtained from somatic cells after exposure to hES-specific transcription factors such as SOX2, KLF4, OCT4, MYC, or NANOG, LIN28, OCT4, and SOX2 or other means that restore aged somatic differentiated cells to pluripotency.
hPS Cells—human pluripotent stem cells such as hES cells, hiPS cells, EC cells, and human parthenogenic stem cells.
HSE—“Human skin equivalents” are mixtures of cells and biological or synthetic matrices manufactured for testing purposes or for therapeutic application in promoting wound repair.
iCM—Induced Cancer Maturation.
iPS Cells—“Induced pluripotent stem cells” are cells with properties similar to hES cells obtained from somatic cells after exposure to ES-specific transcription factors such as SOX2, KLF4, OCT4, MYC, or NANOG, LIN28, OCT4, and SOX2, SOX2, KLF4, OCT4, MYC, and (LIN28A or LIN28B), or other combinations of OCT4, SOX2, KLF4, NANOG, ESRRB, NR5A2, CEBPA, MYC, LIN28A and LIN28B or other factors capable of reversing the developmental aging of differentiated cells back to a pluripotent stem cell state essentially matching the gene expression profile of hES cells.
iS-CSC—“Induced Senolysis of Cancer Stem Cells” refers to the treatment of cells in malignant tumors that are refractory to ablation by chemotherapeutic agents or radiation therapy wherein said iS-CSC treatment causes said refractory cells to revert to a pre-fetal pattern of gene expression and become sensitive to chemotherapeutic agents or radiation therapy.
iTM—Induced Tissue Maturation
iTR—Induced Tissue Regeneration
MEM—Minimal essential medium
MSC—Mesenchymal stem cell
NCs—Neuronal cells, such as the cells of the CNS and peripheral nervous systems including neurons and glial cells such as astocytes and oligodendrocytes.
NT—Neonatal Transition
PBS—Phosphate buffered saline
PCs—Pluripotent stem cells
PCDH—Clustered Protocadherin
PCR—Polymerase Chain Reaction
PS fibroblasts—“Pre-scarring fibroblasts” are fibroblasts derived from the skin of early gestational skin or derived from ED cells that display a prenatal pattern of gene expression in that they promote the rapid healing of dermal wounds without scar formation.
PT—Pluripotency Transition
qRT-PCR—quantitative Real-Time PCR
RFU—Relative Fluorescence Units
RNAi—RNA Interference
RNA-seq—RNA sequencing
SC—Sarcoma Cells
SFM—Serum-Free Medium
siRNA—Small interfering RNA
St. Dev.—Standard Deviation
TR—Tissue Regeneration

### Definitions

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein, the term “differentiated cells” when used in reference to cells having reduced potential to differentiate when compared to the parent pluripotent stem cells. Although, cells with a reduced potential to differentiate can further differentiate if not terminally differentiated.

The term “at least” prior to a number or series of numbers (e.g., “at least two”) is understood to include the number adjacent to the term “at least”, and all subsequent numbers or integers that could logically be included, as clear from context. When “at least” is present before a series of numbers or a range, it is understood that “at least” can modify each of the numbers in the series or range.

As used herein, “up to” as in “up to 10” is understood as up to and including 10, i.e., 0, 1, 2, 3, 4, 5, 6, 7, 8, 9, or 10.

The term “administer” as used herein mean to give or to apply. The term “administering” as used herein includes in vivo administration, as well as administration directly to tissue ex vivo. “Administering” may be accomplished by any route as disclosed below.

The term “antibody” as used herein refers to a polypeptide or group of polypeptides comprised of at least one binding domain that is formed from the folding of polypeptide chains having a binding surface complementary to the features of the antigenic determinant of an antigen.

The basic structural unit of an antibody consists of four polypeptide chains, two identical light chains (L) and two identical heavy chains (H). An antibody may be an oligoclonal antibody, a polyclonal antibody, a monoclonal antibody, a chimeric antibody, a CDR-grafted antibody, a multi-specific antibody, a bi-specific antibody, a catalytic antibody, a humanized antibody, a fully human antibody, and anti-idiotypic antibody, as well as fragments, variants, or derivatives thereof, either alone or in combination with other amino acid sequences provided by known techniques.

As used herein, the term “antigen” refers to a substance that elicits an immune response.

The term “antigen binding site” as used herein refers to the site at the tip of each arm of an antibody that makes physical contact with an antigen and binds it noncovalently. The antigen specificity of the antigen-binding site is determined by its shape and the amino acids present.

The term “alpha or beta CPL isoform” refers to one of the genes or products encoded by the alpha cluster genes: PCDHA1, PCDHA2, PCDHA3, PCDHA4, PCDHA5, PCDHA6, PCDHA7, PCDHA8, PCDHA9, PCDHA10, or PCDHA11, or the beta cluster genes: PCDHB1, PCDHB2, PCDHB3, PCDHB4, PCDHB5, PCDHB6, PCDHB7, PCDHB8, PCDHB9, PCDHB10, PCDHB11, PCDHB12, PCDHB13, PCDHB14, PCDHB15, PCDHB17P, PCDHB18P, or PCDHB19P.

The term “anti-cancer vaccine” or “vaccine” as used herein refer to nucleic acids, e.g., mRNA, or polypeptides, e.g., proteins or fragments thereof, which induce an immune response to cancer cells in the subject. For example, mRNA encoding an antigen identified to be present on the cancer cells of a subject can be administered to the subject to induce an immune response. In some embodiments, the anti-cancer vaccine is mRNA encoding one or more isoforms of the alpha protocadherin cluster and/or the beta protocadherin cluster. In some embodiments, the mRNA encodes PCDHA3 and/or PCDHB3. Another example, polypeptides, or fragments thereof, can be administered to the subject, wherein the polypeptides, or fragments thereof, are one or more isoforms of the alpha protocadherin cluster and/or the beta protocadherin cluster. In some embodiments, the anti-cancer vaccine is polypeptides, or fragments thereof, of one or more isoforms of the alpha protocadherin cluster and/or the beta protocadherin cluster. In some embodiments, the polypeptides, or fragments thereof, are PCDHA3 and/or PCDHB3.

The term “biomarker” or “biosignature” as used herein refers to peptides, proteins, nucleic acids, antibodies, genes, metabolites, or any other substance used as indicators of a biologic state. It is a characteristic that is measured objectively and evaluated as a cellular or molecular indicator of normal biologic processes, pathogenic processes, or pharmalogic responses to a therapeutic composition. The term “indicator” as used herein refers to any substance, number or ratio derived from a series of observed facts that may reveal relative changes as a function of time. A biomarker may be used to diagnose disease risk, presence of disease, or determine treatments for the disease in an individual.

The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

### Chimeric Antigen Receptor Cells

As used herein, the term “chimeric antigen receptor T cell” or “CAR-T cell” refer to engineered T cells expressing a chimeric antigen receptor or CAR.

As used herein, the term “Chimeric antigen receptor” or “CAR” refers to engineered receptors, expressed in immune cells, e.g., T cells, for antigen specificity based on the expressed CAR. The CARs comprise an antigen binding domain also known as antigen targeting region, an extracellular spacer domain or hinge region, a transmembrane domain and at least one intracellular signaling domain or a least one co-stimulatory domain and at least one intracellular signaling domain.

In general, a CAR may comprise an extracellular domain (extracellular part) comprising the antigen binding domain, a transmembrane domain and an intracellular signaling domain. The extracellular domain may be linked to the transmembrane domain by a linker. The extracellular domain may also comprise a signal peptide.

A “signal peptide” refers to a peptide sequence that directs the transport and localization of the protein within a cell, e.g. to a certain cell organelle (such as the endoplasmic reticulum) and/or the cell surface.

An “antigen binding domain” refers to the region of the CAR that specifically binds to an antigen (and thereby is able to target a cell containing an antigen). The CARS may comprise one or more antigen binding domains. Generally, the targeting regions on the CAR are extracellular. The antigen binding domain may comprise an antibody or a fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain.

The term “contact” and its various grammatical forms as used herein refers to a state or condition of touching or of immediate local proximity.

The term “derived from” as used herein refers to any method for receiving, obtaining, or modifying something from a source or origin.

The term “effective amount” is used herein to include the amount of an agent that, when administered to a subject for treating a subject having a disease or disorder (e.g., cancer) is sufficient to effect treatment of the disease (e.g., by diminishing, ameliorating or maintaining the existing disease or one or more symptoms of disease or its related disorders). The “effective amount” may vary depending on the agent, how it is administered, the disease and its severity and the history, age, weight, family history, genetic makeup, stage of pathological processes, the types of preceding or concomitant treatments, if any, and other individual characteristics of the subject to be treated. It is generally preferred that a maximum dose be used, that is, the highest safe dose according to some medical judgment. The terms “dose” and “dosage” are used interchangeably herein.

As used herein, the term “immune cell” or “immune effector cell” refers to a cell that may be part of the immune system and executes a particular effector function such as alpha-beta T cells, NK cells, NKT cells, B cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes or macrophages. Preferred immune cells are cells with cytotoxic effector function such as alpha-beta T cells, NK cells, NKT cells, ILC, CIK cells, LAK cells or gamma-delta T cells. “Effector function” means a specialized function of a cell, e.g. in a T cell an effector function may be cytolytic activity or helper activity including the secretion of cytokines.

As used herein, the term “immune response” refers to an integrated bodily response to an antigen and preferably refers to a cellular immune response or a cellular as well as a humoral immune response. The immune response may be protective, preventive, prophylactic, and/or therapeutic.

As used herein, the term “inducing an immune response” may mean that there was no immune response against a particular antigen before induction, but it may also mean that there was a certain level of immune response against a particular antigen before induction and after induction said immune response is enhanced. Thus, “inducing an immune response” also includes “enhancing an immune response”. Preferably, after inducing an immune response in a subject, said subject is protected from developing a disease such as a cancer disease or the disease condition is ameliorated by inducing an immune response.

As used herein, the term “immunoglobulin superfamily member” is a protein with an immunoglobulin (Ig) domain and include antibodies such as IgA, IgD, IgE, IgG, IgM, nanobodies, T-cell receptors, co-receptor molecules such as CD4, CD8, and CD19, co-stimulatory molecules such as CD28, natural killer cell receptors such as killer cell immunoglobulin-like receptor (KIL), and antigen receptor accessory molecules such as CD3 and CD79a and CD79b. As used herein, the term “nucleic acid” is deoxyribonucleic acid (DNA) or ribonucleic acid (RNA), more preferably RNA, and can be in vitro transcribed RNA (IVT RNA) or synthetic RNA. Nucleic acids include according to the invention genomic DNA, cDNA, mRNA, recombinantly produced and chemically synthesized molecules.

As used herein, the term “protocadherins” refers to a large subfamily of cadherin polypeptides, calcium-dependent cell adhesion molecules. Protocadherins are subdivided into clustered and non-clustered protocadherins and involved in development and disease (e.g., cancer).

As used herein, the term “subject” includes any mammal, preferably a human.

As used herein, the terms “T cell” and “T lymphocyte” are used interchangeably herein and include T helper cells (CD4+ T cells) and cytotoxic T cells (CTLs, CD8+ T cells) which comprise cytolytic T cells. T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptor (TCR). The thymus is the principal organ responsible for the maturation of T cells. Several different subsets of T cells have been discovered, each with a distinct function. The term “analytical reprogramming technology” refers to a variety of methods to reprogram the pattern of gene expression of a somatic cell to that of a more pluripotent state, such as that of an iPS, ES, ED, EC or EG cell, wherein the reprogramming occurs in multiple and discrete steps and does not rely simply on the transfer of a somatic cell into an oocyte and the activation of that oocyte (see PCT Patent Application Ser. Nos. PCT/US02/37899 and PCT/US06/30632, the disclosure of each of which is incorporated by reference herein in its entirety).

The term “blastomere/morula cells” refers to blastomere or morula cells in a mammalian embryo or blastomere or morula cells cultured in vitro with or without additional cells including differentiated derivatives of those cells.

The term “cancer maturation” refers to the alteration of gene expression in premalignant or malignant cancer cells such that said premalignant or malignant cancer cells that initially express markers of embryonic cells, are altered to express markers of fetal or adult cells.

The term “cell expressing gene X”, “gene X is expressed in a cell” (or cell population), or equivalents thereof, means that analysis of the cell using a specific assay platform provided a positive result. The converse is also true (i.e., by a cell not expressing gene X, or equivalents, is meant that analysis of the cell using a specific assay platform provided a negative result). Thus, any gene expression result described herein is tied to the specific probe or probes employed in the assay platform (or platforms) for the gene indicated.

The term “cell line” refers to a mortal or immortal population of cells that is capable of propagation and expansion in vitro.

The term “cellular reconstitution” refers to the transfer of a nucleus of chromatin to cellular cytoplasm so as to obtain a functional cell.

The term “clonal” refers to a population of cells obtained the expansion of a single cell into a population of cells all derived from that original single cells and not containing other cells.

The term “clonal embryonic progenitor cells” refers to embryonic progenitor cells that derived in vitro from a single cell.

The term “cytoplasmic bleb” refers to the cytoplasm of a cell bound by an intact or permeabilized but otherwise intact plasma membrane, but lacking a nucleus.

The term “DNA damaging agent” refers to a therapeutic agent that induces DNA breaks in the genome. In some embodiments, the DNA damaging agent is high dose platinum-based alkylating chemotherapy, platinum compounds, thiotepa, cyclophosphamide, iphosphamide, nitrosureas, nitrogen mustard derivatives, mitomycins, epipodophyllotoxins, camptothecins, anthracyclines, poly(ADP-ribose) polymerase (PARP) inhibitors, ionizing radiation, ABT-888, olaparib (AZT-2281), gemcitabine, CEP-9722, AG014699, AG014699 with Temozolomide, and BSI-201.

As used herein, the term “differentiation” and its various grammatical forms refers to the process by which an immature cell because specialized in order to perform a specific function. The term “differentiated cells” when used in reference to cells made by methods of this invention from pluripotent stem cells refer to cells having reduced potential to differentiate when compared to the parent pluripotent stem cells. The differentiated cells of this invention comprise cells that could differentiate further (i.e., they may not be terminally differentiated).

The term “embryonic” as used herein refers to the state of the differentiation of mammalian cells wherein the cell possess a scarless regenerative phenotype which therefore distinguishes them from that of the cells of the same differentiated type but in in a fetal or adult non-regenerative state of development that have little to no capacity for scarless regeneration. The term “embryonic” generally refers to development up to approximately Carnegie Stage 23, however, depending upon the tissue, may occur later in development. Excluded from the definition are cell types capable of scarless regeneration in the adult state such as hepatocytes and blood cells. In the case of the human species, the transition from embryonic to fetal development occurs at about 8 weeks of prenatal development, in mouse it occurs on or about 16 days, and in the rat species, at approximately 17.5 days post coitum. (see, the web site: www.php.med.unsw.edu. au/embryology/index.php?title=Mouse_Timeline_Detailed).

In some embodiments, genes expressed in embryonic cells include, but are not limited to, SOX2, KLF4, OCT4, MYC, NANOG, LIN28A, LIN28B, ESRRB, NR5A2, TERT, SSEA, TRA, and CEBPA. In some embodiments, genes repressed in embryonic cells include, but are not limited to, COX7A1.

The term “embryonic pattern of CPL isoform expression” refers to a pattern of gene expression characterized by activation of members of the α and β clusters and repression of members of the γ locus with the exception of PCDHGB4 and PCDHGB6 which are relatively highly expressed in embryonic cells. In some embodiments, expression one or more members of the α cluster is elevated in embryonic cells, e.g., PCDHA2, PCDHA4, PCDHA10, and PCDHA12. In some embodiments, expression one or more members of the β cluster is elevated in embryonic cells, e.g., PCDHB2, PCDHB5, PCDHB9, PCDHB10, PCDHB13, PCDHB14, and PCDHB16. In some embodiments, expression one or more members of the γ cluster is elevated in embryonic cells, e.g., PCDHGB4 and PCDHGB6.

The term “fetal-adult pattern of CPL isoform expression” or “adult pattern of CPL isoform expression” refers to a pattern of gene expression characterized by activation of expression of members of the α and β clusters and decreased expression of members of the γ locus with the exception of PCDHGB4 and PCDHGB6 which are relatively highly expressed in embryonic cells.

The term “embryonic progenitor cells” refers to cells of all somatic cell lineages that are more differentiated than pluripotent stem cells (e.g. embryonic stem cells) but have not matured so as to express markers of fetal or adult cell types. In the case of human embryonic progenitor cells, they would express markers of cells of less than eight weeks of gestation, such as relatively low to no expression of COX7A1 compared to fetal or adult-derived cells.

The term “embryonic stem cells” (ES cells) refers to cells derived from the inner cell mass of blastocysts, blastomeres, or morulae that have been serially passaged as cell lines while maintaining an undifferentiated state (e.g. expressing TERT, OCT4, and SSEA and TRA antigens specific for ES cells of the species). The ES cells may be derived from fertilization of an egg cell with sperm or DNA, nuclear transfer, parthenogenesis, or by means to generate hES cells with hemizygosity or homozygosity in the MHC region. While ES cells have historically been defined as cells capable of differentiating into all of the somatic cell types as well as germ line when transplanted into a preimplantation embryo, candidate ES cultures from many species, including human, have a more flattened appearance in culture and typically do not contribute to germ line differentiation, and are therefore called “ES-like cells.” It is commonly believed that human ES cells are in reality “ES-like”, however, in this application we will use the term ES cells to refer to both ES and ES-like cell lines. The ES cells may be derived from fertilization of an egg cell with sperm or DNA, nuclear transfer, parthenogenesis, or by means to generate hES cells with hemizygosity or homozygosity in the MHC region. The term “human embryonic stem cells” (hES cells) refers to human ES cells.

The term “global modulator of TR” or “global modulator of iTR” refers to agents capable of modulating a multiplicity of iTR genes or iTM genes including, but not limited to, agents capable of downregulating COX7A1 while simultaneously up-regulating PCDHB2, or down-regulating NAALADL1 while simultaneously up-regulating AMH in cells derived from fetal or adult sources and are capable of inducing a pattern of gene expression leading to increased scarless tissue regeneration in response to tissue damage or degenerative disease.

The term “human induced pluripotent stem cells” refers to cells with properties similar to hES cells, including the ability to form all three germ layers when transplanted into immunocompromised mice wherein said iPS cells are derived from cells of varied somatic cell lineages following exposure to de-differentiation factors, for example hES cell-specific transcription factor combinations: KLF4, SOX2, MYC; OCT4 or SOX2, OCT4, NANOG, and LIN28; or various combinations of OCT4, SOX2, KLF4, NANOG, ESRRB, NR5A2, CEBPA, MYC, LIN28A and LIN28B or other methods that induce somatic cells to attain a pluripotent stem cell state with properties similar to hES cells. However, the reprogramming of somatic cells by somatic cell nuclear transfer (SCNT) are typically referred to as NT-ES cells as opposed to iPS cells.

The term “induced Cancer Maturation” or “iCM” refers to methods resulting in a change in the phenotype of premalignant or malignant cells such that subsequent to said induction, the cells express markers normally expressed in that cell type in fetal or adult stages of development as opposed to the embryonic stages. The term as used herein, is synonymous with epithelial-mesenchymal transition (EMT) when applied carcinomas and adenocarcinomas.

The term “induced tissue regeneration” refers to the use of the methods of the present invention as well as the methods disclosed in (see PCT Patent Application Ser. No. PCT/US2014/040601 and U.S. patent application Ser. No. 14/896,664) to alter the molecular composition of fetal or adult mammalian cells such that said cells are capable or regenerating functional tissue following damage to that tissue wherein said regeneration would not be the normal outcome in animals of that species. The term “iCM factors” refers to molecules that alter the levels of CM activators and CM inhibitors in a manner leading to CM in a tumor for therapeutic effect. The term “iCM genes” refers to genes that when altered in expression can cause CM in a tumor for therapeutic effect.

The term “isolated” refers to a substance that is (i) separated from at least some other substances with which it is normally found in nature, usually by a process involving the hand of man, (ii) artificially produced (e.g., chemically synthesized), and/or (iii) present in an artificial environment or context (i.e., an environment or context in which it is not normally found in nature).

The term “iTR factors” refers to molecules that alter the levels of TR activators and TR inhibitors in a manner leading to TR in a tissue not naturally capable of TR.

The term “iTR genes” refers to genes that when altered in expression can cause induced tissue regeneration in tissues not normally capable of such regeneration.

The term “nucleic acid” is used interchangeably with “polynucleotide” and encompasses in various embodiments naturally occurring polymers of nucleosides, such as DNA and RNA, and non-naturally occurring polymers of nucleosides or nucleoside analogs. In some embodiments a nucleic acid comprises standard nucleosides (abbreviated A, G, C, T, U). In other embodiments a nucleic acid comprises one or more non-standard nucleosides. In some embodiments, one or more nucleosides are non-naturally occurring nucleosides or nucleotide analogs. A nucleic acid can comprise modified bases (for example, methylated bases), modified sugars (2′-fluororibose, arabinose, or hexose), modified phosphate groups or other linkages between nucleosides or nucleoside analogs (for example, phosphorothioates or 5′-N-phosphoramidite linkages), locked nucleic acids, or morpholinos. In some embodiments, a nucleic acid comprises nucleosides that are linked by phosphodiester bonds, as in DNA and RNA. In some embodiments, at least some nucleosides are linked by non-phosphodiester bond(s). A nucleic acid can be single-stranded, double-stranded, or partially double-stranded. An at least partially double-stranded nucleic acid can have one or more overhangs, e.g., 5′ and/or 3′ overhang(s). Nucleic acid modifications (e.g., nucleoside and/or backbone modifications, including use of non-standard nucleosides) known in the art as being useful in the context of RNA interference (RNAi), aptamer, or antisense-based molecules for research or therapeutic purposes are contemplated for use in various embodiments of the instant invention. See, e.g., Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008. In some embodiments, a modification increases half-life and/or stability of a nucleic acid, e.g., in vivo, relative to RNA or DNA of the same length and strandedness. In some embodiments, a modification decreases immunogenicity of a nucleic acid relative to RNA or DNA of the same length and strandedness. In some embodiments, between 5% and 95% of the nucleosides in one or both strands of a nucleic acid is modified. Modifications may be located uniformly or nonuniformly, and the location of the modifications (e.g., near the middle, near or at the ends, alternating, etc.) can be selected to enhance desired propert(ies). A nucleic acid may comprise a detectable label, e.g., a fluorescent dye, radioactive atom, etc. “Oligonucleotide” refers to a relatively short nucleic acid, e.g., typically between about 4 and about 60 nucleotides long. Where reference is made herein to a polynucleotide, it is understood that both DNA, RNA, and in each case both single- and double-stranded forms (and complements of each single-stranded molecule) are provided.

“Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “oligoclonal” refers to a population of cells that originated from a small population of cells, typically 2-1000 cells, that appear to share similar characteristics such as morphology or the presence or absence of markers of differentiation that differ from those of other cells in the same culture. Oligoclonal cells are isolated from cells that do not share these common characteristics, and are allowed to proliferate, generating a population of cells that are essentially entirely derived from the original population of similar cells.

The term “pluripotent stem cells” refers to animal cells capable of differentiating into more than one differentiated cell type. Such cells include hES cells, blastomere/morula cells and their derived hED cells, hiPS cells, hEG cells, hEC cells, and adult-derived cells including mesenchymal stem cells, neuronal stem cells, and bone marrow-derived stem cells. Pluripotent stem cells may be genetically modified or not genetically modified. Genetically modified cells may include markers such as fluorescent proteins to facilitate their identification within the egg.

The term “polypeptide” refers to a polymer of amino acids. The terms “protein” and “polypeptide” are used interchangeably herein. A peptide is a relatively short polypeptide, typically between about 2 and 60 amino acids in length. Polypeptides used herein typically contain the standard amino acids (i.e., the 20 L-amino acids that are most commonly found in proteins). However, a polypeptide can contain one or more non-standard amino acids (which may be naturally occurring or non-naturally occurring) and/or amino acid analogs known in the art in certain embodiments. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Polypeptides may be purified from natural sources, produced using recombinant DNA technology, synthesized through chemical means such as conventional solid phase peptide synthesis, etc.

The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated. A polypeptide may be cyclic or contain a cyclic portion. Where a naturally occurring polypeptide is discussed herein, it will be understood that the invention encompasses embodiments that relate to any isoform thereof (e.g., different proteins arising from the same gene as a result of alternative splicing or editing of mRNA or as a result of different alleles of a gene, e.g., alleles differing by one or more single nucleotide polymorphisms (typically such alleles will be at least 95%, 96%, 97%, 98%, 99%, or more identical to a reference or consensus sequence). A polypeptide may comprise a sequence that targets it for secretion or to a particular intracellular compartment (e.g., the nucleus) and/or a sequence targets the polypeptide for post-translational modification or degradation. Certain polypeptides may be synthesized as a precursor that undergoes post-translational cleavage or other processing to become a mature polypeptide. In some instances, such cleavage may only occur upon particular activating events. Where relevant, the invention provides embodiments relating to precursor polypeptides and embodiments relating to mature versions of a polypeptide.

The term “prenatal” refers to a stage of embryonic development of a placental mammal prior to which an animal is not capable of viability apart from the uterus.

The term “primordial stem cells” refers collectively to pluripotent stem cells capable of differentiating into cells of all three primary germ layers: endoderm, mesoderm, and ectoderm, as well as neural crest. Therefore, examples of primordial stem cells would include but not be limited by human or non-human mammalian ES cells or cell lines, blastomere/morula cells and their derived ED cells, iPS, and EG cells.

The term “purified” refers to agents or entities (e.g., compounds) that have been separated from most of the components with which they are associated in nature or when originally generated. In general, such purification involves action of the hand of man. Purified agents or entities may be partially purified, substantially purified, or pure. Such agents or entities may be, for example, at least 50%, 60%, 70%, 75%, 80%, 85%, 90%, 95%, 96%, 97%, 98%, 99%, or more than 99% pure. In some embodiments, a nucleic acid or polypeptide is purified such that it constitutes at least 75%, 80%, 855%, 90%, 95%, 96%, 97%, 98%, 99%, or more, of the total nucleic acid or polypeptide material, respectively, present in a preparation. Purity can be based on, e.g., dry weight, size of peaks on a chromatography tracing, molecular abundance, intensity of bands on a gel, or intensity of any signal that correlates with molecular abundance, or any art-accepted quantification method. In some embodiments, water, buffers, ions, and/or small molecules (e.g., precursors such as nucleotides or amino acids), can optionally be present in a purified preparation. A purified molecule may be prepared by separating it from other substances (e.g., other cellular materials), or by producing it in such a manner to achieve a desired degree of purity. In some embodiments, a purified molecule or composition refers to a molecule or composition that is prepared using any art-accepted method of purification. In some embodiments “partially purified” means that a molecule produced by a cell is no longer present within the cell, e.g., the cell has been lysed and, optionally, at least some of the cellular material (e.g., cell wall, cell membrane(s), cell organelle(s)) has been removed.

The terms “small interfering RNA” (siRNA) and “RNA interference” (RNAi) are used interchangeably and otherwise consistently with its meaning in the art to refer to a phenomenon whereby double-stranded RNA (dsRNA) triggers the sequence-specific degradation or translational repression of a corresponding mRNA having complementarity to a strand of the dsRNA. It will be appreciated that the complementarity between the strand of the dsRNA and the mRNA need not be 100% but need only be sufficient to mediate inhibition of gene expression (also referred to as “silencing” or “knockdown”). For example, the degree of complementarity is such that the strand can either (i) guide cleavage of the mRNA in the RNA-induced silencing complex (RISC); or (ii) cause translational repression of the mRNA. In certain embodiments the double-stranded portion of the RNA is less than about 30 nucleotides in length, e.g., between 17 and 29 nucleotides in length. In certain embodiments a first strand of the dsRNA is at least 80%, 85%, 90%, 95%, or 100% complementary to a target mRNA and the other strand of the dsRNA is at least 80%, 85%, 90%, 95%, or 100% complementary to the first strand. In mammalian cells, RNAi may be achieved by introducing an appropriate double-stranded nucleic acid into the cells or expressing a nucleic acid in cells that is then processed intracellularly to yield dsRNA therein. Nucleic acids capable of mediating RNAi are referred to herein as “RNAi agents”. Exemplary nucleic acids capable of mediating RNAi are a short hairpin RNA (shRNA), a short interfering RNA (siRNA), and a microRNA precursor. These terms are well known and are used herein consistently with their meaning in the art. siRNAs typically comprise two separate nucleic acid strands that are hybridized to each other to form a duplex. They can be synthesized in vitro, e.g., using standard nucleic acid synthesis techniques. siRNAs are typically double-stranded oligonucleotides having 16-30, e.g., 16, 17, 18, 19, 20, 21, 22, 23, 24, 25, 26, 27, 28, 29, or 30 nucleotides (nt) in each strand, wherein the double-stranded oligonucleotide comprises a double-stranded portion between 15 and 29 nucleotides long and either or both of the strands may comprise a 3′ overhang between, e.g., 1-5 nucleotides long, or either or both ends can be blunt. In some embodiments, an siRNA comprises strands between 19 and 25 nt, e.g., between 21 and 23 nucleotides long, wherein one or both strands comprises a 3′ overhang of 1-2 nucleotides. One strand of the double-stranded portion of the siRNA (termed the “guide strand” or “antisense strand”) is substantially complementary (e.g., at least 80% or more, e.g., 85%, 90%, 95%, or 100%) complementary to (e.g., having 3, 2, 1, or 0 mismatched nucleotide(s)) a target region in the mRNA, and the other double-stranded portion is substantially complementary to the first double-stranded portion. In many embodiments, the guide strand is 100% complementary to a target region in an mRNA and the other passenger strand is 100% complementary to the first double-stranded portion (it is understood that, in various embodiments, the 3′ overhang portion of the guide strand, if present, may or may not be complementary to the mRNA when the guide strand is hybridized to the mRNA). In some embodiments, a shRNA molecule is a nucleic acid molecule comprising a stem-loop, wherein the double-stranded stem is 16-30 nucleotides long and the loop is about 1-10 nucleotides long. siRNA can comprise a wide variety of modified nucleosides, nucleoside analogs and can comprise chemically or biologically modified bases, modified backbones, etc. Without limitation, any modification recognized in the art as being useful for RNAi can be used. Some modifications result in increased stability, cell uptake, potency, etc. Some modifications result in decreased immunogenicity or clearance. In certain embodiments the siRNA comprises a duplex about 19-23 (e.g., 19, 20, 21, 22, or 23) nucleotides in length and, optionally, one or two 3′ overhangs of 1-5 nucleotides in length, which may be composed of deoxyribonucleotides. shRNA comprise a single nucleic acid strand that contains two complementary portions separated by a predominantly non-selfcomplementary region. The complementary portions hybridize to form a duplex structure and the non-selfcomplementary region forms a loop connecting the 3′ end of one strand of the duplex and the 5′ end of the other strand. shRNAs undergo intracellular processing to generate siRNAs. Typically, the loop is between 1 and 8, e.g., 2-6 nucleotides long.

MicroRNAs (miRNAs) are small, naturally occurring, non-coding, single-stranded RNAs of about 21-25 nucleotides (in mammalian systems) that inhibit gene expression in a sequence-specific manner. They are generated intracellularly from precursors (pre-miRNA) having a characteristic secondary structure comprised of a short hairpin (about 70 nucleotides in length) containing a duplex that often includes one or more regions of imperfect complementarity which is in turn generated from a larger precursor (pri-miRNA). Naturally occurring miRNAs are typically only partially complementary to their target mRNA and often act via translational repression. RNAi agents modelled on endogenous miRNA or miRNA precursors are of use in certain embodiments of the invention. For example, an siRNA can be designed so that one strand hybridizes to a target mRNA with one or more mismatches or bulges mimicking the duplex formed by a miRNA and its target mRNA. Such siRNA may be referred to as miRNA mimics or miRNA-like molecules. miRNA mimics may be encoded by precursor nucleic acids whose structure mimics that of naturally occurring miRNA precursors.

In certain embodiments an RNAi agent is a vector (e.g., a plasmid or virus) that comprises a template for transcription of an siRNA (e.g., as two separate strands that can hybridize to each other), shRNA, or microRNA precursor. Typically the template encoding the siRNA, shRNA, or miRNA precursor is operably linked to expression control sequences (e.g., a promoter), as known in the art. Such vectors can be used to introduce the template into vertebrate cells, e.g., mammalian cells, and result in transient or stable expression of the siRNA, shRNA, or miRNA precursor. Precursors (shRNA or miRNA precursors) are processed intracellularly to generate siRNA or miRNA.

In general, small RNAi agents such as siRNA can be chemically synthesized or can be transcribed in vitro or in vivo from a DNA template either as two separate strands that then hybridize, or as an shRNA which is then processed to generate an siRNA. Often RNAi agents, especially those comprising modifications, are chemically synthesized. Chemical synthesis methods for oligonucleotides are well known in the art.

The term “small molecule” as used herein, is an organic molecule that is less than about 2 kilodaltons (KDa) in mass. In some embodiments, the small molecule is less than about 1.5 KDa, or less than about 1 KDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups. In some embodiments, a small molecule is non-polymeric. In some embodiments, a small molecule is not an amino acid. In some embodiments, a small molecule is not a nucleotide. In some embodiments, a small molecule is not a saccharide.

The term “subject” can be any multicellular animal. Often a subject is a vertebrate, e.g., a mammal or avian. Exemplary mammals include, e.g., humans, non-human primates, rodents (e.g., mouse, rat, rabbit), ungulates (e.g., ovine, bovine, equine, caprine species), canines, and felines. Often, a subject is an individual to whom a compound is to be delivered, e.g., for experimental, diagnostic, and/or therapeutic purposes or from whom a sample is obtained or on whom a diagnostic procedure is performed (e.g., a sample or procedure that will be used to assess tissue damage and/or to assess the effect of a compound of the invention).

The term “tissue damage” is used herein to refer to any type of damage or injury to cells, tissues, organs, or other body structures. The term encompasses, in various embodiments, degeneration due to disease, damage due to physical trauma or surgery, damage caused by exposure to deleterious substance, and other disruptions in the structure and/or functionality of cells, tissues, organs, or other body structures.

The term “tissue regeneration” or “TR” refers to at least partial regeneration, replacement, restoration, or regrowth of a tissue, organ, or other body structure, or portion thereof, following loss, damage, or degeneration, where said tissue regeneration but for the methods described in the present invention would not take place. Examples of tissue regeneration include the regrowth of severed digits or limbs including the regrowth of cartilage, bone, muscle, tendons, and ligaments, the scarless regrowth of bone, cartilage, skin, or muscle that has been lost due to injury or disease, with an increase in size and cell number of an injured or diseased organ such that the tissue or organ approximates the normal size of the tissue or organ or its size prior to injury or disease. Depending on the tissue type, tissue regeneration can occur via a variety of different mechanisms such as, for example, the rearrangement of pre-existing cells and/or tissue (e.g., through cell migration), the division of adult somatic stem cells or other progenitor cells and differentiation of at least some of their descendants, and/or the dedifferentiation, transdifferentiation, and/or proliferation of cells.

The term “TR activator genes” refers to genes whose lack of expression in fetal and adult cells but whose expression in embryonic phases of development facilitate TR.

The term “TR inhibitor genes” refers to genes whose expression in fetal and adult animals inhibit TR.

The term “treat”, “treating”, “therapy”, “therapeutic” and similar terms in regard to a subject refer to providing medical and/or surgical management of the subject. Treatment can include, but is not limited to, administering a compound or composition (e.g., a pharmaceutical composition) to a subject. Treatment of a subject according to the instant invention is typically undertaken in an effort to promote regeneration, e.g., in a subject who has suffered tissue damage or is expected to suffer tissue damage (e.g., a subject who will undergo surgery). The effect of treatment can generally include increased regeneration, reduced scarring, and/or improved structural or functional outcome following tissue damage (as compared with the outcome in the absence of treatment), and/or can include reversal or reduction in severity or progression of a degenerative disease.

The term “variant” as applied to a particular polypeptide refers to a polypeptide that differs from such polypeptide (sometimes referred to as the “original polypeptide”) by one or more amino acid alterations, e.g., addition(s), deletion(s), and/or substitution(s). Sometimes an original polypeptide is a naturally occurring polypeptide (e.g., from human or non-human animal) or a polypeptide identical thereto. Variants may be naturally occurring or created using, e.g., recombinant DNA techniques or chemical synthesis. An addition can be an insertion within the polypeptide or an addition at the N- or C-terminus. In some embodiments, the number of amino acids substituted, deleted, or added can be for example, about 1 to 30, e.g., about 1 to 20, e.g., about 1 to 10, e.g., about 1 to 5, e.g., 1, 2, 3, 4, or 5. In some embodiments, a variant comprises a polypeptide whose sequence is homologous to the sequence of the original polypeptide over at least 50 amino acids, at least 100 amino acids, at least 150 amino acids, or more, up to the full length of the original polypeptide (but is not identical in sequence to the original polypeptide), e.g., the sequence of the variant polypeptide is at least 50%, 60%, 70%, 75%, 80%, 85%, 90%, 95%, 96%, 97%, 98%, 99%, or more identical to the sequence of the original polypeptide over at least 50 amino acids, at least 100 amino acids, at least 150 amino acids, or more, up to the full length of the original polypeptide. In some embodiments, a variant comprises a polypeptide at least 50%, 60%, 70%, 75%, 80%, 85%, 90%, 91%, 92%, 93%, 94%, 95%, 96%, 97%, 98%, 99%, 99.5% or more identical to an original polypeptide over at least 50%, 60%, 70%, 80%, 85%, 90%, 91%, 92%, 93%, 94%, 95%, 96%, 97%, 98%, 99%, or 100% of the length of the original polypeptide. In some embodiments a variant comprises at least one functional or structural domain, e.g., a domain identified as such in the Conserved Domain Database (CDD) of the National Center for Biotechnology Information (www.ncbi.nih.gov), e.g., an NCBI-curated domain.

In some embodiments one, more than one, or all biological functions or activities of a variant or fragment is substantially similar to that of the corresponding biological function or activity of the original molecule. In some embodiments, a functional variant retains at least 10%, 20%, 30%, 40%, 50%, 60%, 70%, 80%, 90%, 95%, 96%, 97%, 98%, 99%, or more of the activity of the original polypeptide, e.g., about equal activity. In some embodiments, the activity of a variant is up to approximately 100%, approximately 125%, or approximately 150% of the activity of the original molecule. In other nonlimiting embodiments an activity of a variant or fragment is considered substantially similar to the activity of the original molecule if the amount or concentration of the variant needed to produce a particular effect is within 0.5 to 5-fold of the amount or concentration of the original molecule needed to produce that effect.

In some embodiments amino acid “substitutions” in a variant are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Within a particular group, certain substitutions may be of particular interest, e.g., replacements of leucine by isoleucine (or vice versa), serine by threonine (or vice versa), or alanine by glycine (or vice versa). Of course non-conservative substitutions are often compatible with retaining function as well. In some embodiments, a substitution or deletion does not alter or delete an amino acid important for activity. Insertions or deletions may range in size from about 1 to 20 amino acids, e.g., 1 to 10 amino acids. In some instances larger domains may be removed without substantially affecting function. In certain embodiments of the invention the sequence of a variant can be obtained by making no more than a total of 5, 10, 15, or 20 amino acid additions, deletions, or substitutions to the sequence of a naturally occurring enzyme. In some embodiments no more than 1%, 5%, 10%, or 20% of the amino acids in a polypeptide are insertions, deletions, or substitutions relative to the original polypeptide. Guidance in determining which amino acid residues may be replaced, added, or deleted without eliminating or substantially reducing activities of interest, may be obtained by comparing the sequence of the particular polypeptide with that of homologous polypeptides (e.g., from other organisms) and minimizing the number of amino acid sequence changes made in regions of high homology (conserved regions) or by replacing amino acids with those found in homologous sequences since amino acid residues that are conserved among various species are more likely to be important for activity than amino acids that are not conserved.

In some embodiments, a variant of a polypeptide comprises a heterologous polypeptide portion. The heterologous portion often has a sequence that is not present in or homologous to the original polypeptide. A heterologous portion may be, e.g., between 5 and about 5,000 amino acids long, or longer. Often it is between 5 and about 1,000 amino acids long. In some embodiments, a heterologous portion comprises a sequence that is found in a different polypeptide, e.g., a functional domain. In some embodiments, a heterologous portion comprises a sequence useful for purifying, expressing, solubilizing, and/or detecting the polypeptide. In some embodiments, a heterologous portion comprises a polypeptide “tag”, e.g., an affinity tag or epitope tag. For example, the tag can be an affinity tag (e.g., HA, TAP, Myc, 6×His, Flag, GST), fluorescent or luminescent protein (e.g., EGFP, ECFP, EYFP, Cerulean, DsRed, mCherry), solubility-enhancing tag (e.g., a SUMO tag, NUS A tag, SNUT tag, or a monomeric mutant of the Ocr protein of bacteriophage T7). See, e.g., Esposito D and Chatterjee D K. Curr Opin Biotechnol.; 17(4):353-8 (2006). In some embodiments, a tag can serve multiple functions. A tag is often relatively small, e.g., ranging from a few amino acids up to about 100 amino acids long. In some embodiments a tag is more than 100 amino acids long, e.g., up to about 500 amino acids long, or more. In some embodiments, a polypeptide has a tag located at the N- or C-terminus, e.g., as an N- or C-terminal fusion. The polypeptide could comprise multiple tags. In some embodiments, a 6.times. His tag and a NUS tag are present, e.g., at the N-terminus. In some embodiments, a tag is cleavable, so that it can be removed from the polypeptide, e.g., by a protease. In some embodiments, this is achieved by including a sequence encoding a protease cleavage site between the sequence encoding the portion homologous to the original polypeptide and the tag. Exemplary proteases include, e.g., thrombin, TEV protease, Factor Xa, PreScission protease, etc. In some embodiments, a “self-cleaving” tag is used. See, e.g., PCT/US05/05763. Sequences encoding a tag can be located 5′ or 3′ with respect to a polynucleotide encoding the polypeptide (or both). In some embodiments a tag or other heterologous sequence is separated from the rest of the polypeptide by a polypeptide linker. For example, a linker can be a short polypeptide (e.g., 15-25 amino acids). Often a linker is composed of small amino acid residues such as serine, glycine, and/or alanine. A heterologous domain could comprise a transmembrane domain, a secretion signal domain, etc.

In certain embodiments of the invention a fragment or variant, optionally excluding a heterologous portion, if present, possesses sufficient structural similarity to the original polypeptide so that when its 3-dimensional structure (either actual or predicted structure) is superimposed on the structure of the original polypeptide, the volume of overlap is at least 70%, preferably at least 80%, more preferably at least 90% of the total volume of the structure of the original polypeptide. A partial or complete 3-dimensional structure of the fragment or variant may be determined by crystallizing the protein, which can be done using standard methods. Alternately, an NMR solution structure can be generated, also using standard methods. A modeling program such as MODELER (Sali, A. and Blundell, T L, J. Mol. Biol., 234, 779-815, 1993), or any other modeling program, can be used to generate a predicted structure. If a structure or predicted structure of a related polypeptide is available, the model can be based on that structure. The PROSPECT-PSPP suite of programs can be used (Guo, J T, et al., Nucleic Acids Res. 32 (Web Server issue):W522-5, Jul. 1, 2004). Where embodiments of the invention relate to variants of a polypeptide, it will be understood that polynucleotides encoding the variant are provided.

The term “vector” is used herein to refer to a nucleic acid or a virus or portion thereof (e.g., a viral capsid or genome) capable of mediating entry of, e.g., transferring, transporting, etc., a nucleic acid molecule into a cell. Where the vector is a nucleic acid, the nucleic acid molecule to be transferred is generally linked to, e.g., inserted into, the vector nucleic acid molecule. A nucleic acid vector may include sequences that direct autonomous replication (e.g., an origin of replication), or may include sequences sufficient to allow integration of part or all of the nucleic acid into host cell DNA. Useful nucleic acid vectors include, for example, DNA or RNA plasmids, cosmids, and naturally occurring or modified viral genomes or portions thereof or nucleic acids (DNA or RNA) that can be packaged into viral) capsids. Plasmid vectors typically include an origin of replication and one or more selectable markers. Plasmids may include part or all of a viral genome (e.g., a viral promoter, enhancer, processing or packaging signals, etc.). Viruses or portions thereof that can be used to introduce nucleic acid molecules into cells are referred to as viral vectors. Useful viral vectors include adenoviruses, adeno-associated viruses, retroviruses, lentiviruses, vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-defective, and such replication-defective viral vectors may be preferable for therapeutic use. Where sufficient information is lacking it may, but need not be, supplied by a host cell or by another vector introduced into the cell. The nucleic acid to be transferred may be incorporated into a naturally occurring or modified viral genome or a portion thereof or may be present within the virus or viral capsid as a separate nucleic acid molecule. It will be appreciated that certain plasmid vectors that include part or all of a viral genome, typically including viral genetic information sufficient to direct transcription of a nucleic acid that can be packaged into a viral capsid and/or sufficient to give rise to a nucleic acid that can be integrated into the host cell genome and/or to give rise to infectious virus, are also sometimes referred to in the art as viral vectors. Vectors may contain one or more nucleic acids encoding a marker suitable for use in the identifying and/or selecting cells that have or have not been transformed or transfected with the vector. Markers include, for example, proteins that increase or decrease either resistance or sensitivity to antibiotics (e.g., an antibiotic-resistance gene encoding a protein that confers resistance to an antibiotic such as puromycin, hygromycin or blasticidin) or other compounds, enzymes whose activities are detectable by assays known in the art (e.g., beta.-galactosidase or alkaline phosphatase), and proteins or RNAs that detectably affect the phenotype of transformed or transfected cells (e.g., fluorescent proteins). Expression vectors are vectors that include regulatory sequence(s), e.g., expression control sequences such as a promoter, sufficient to direct transcription of an operably linked nucleic acid. Regulatory sequences may also include enhancer sequences or upstream activator sequences. Vectors may optionally include 5′ leader or signal sequences. Vectors may optionally include cleavage and/or polyadenylations signals and/or a 3′ untranslated regions. Vectors often include one or more appropriately positioned sites for restriction enzymes, to facilitate introduction into the vector of the nucleic acid to be expressed. An expression vector comprises sufficient cis-acting elements for expression; other elements required or helpful for expression can be supplied by the host cell or in vitro expression system.

Various techniques may be employed for introducing nucleic acid molecules into cells. Such techniques include chemical-facilitated transfection using compounds such as calcium phosphate, cationic lipids, cationic polymers, liposome-mediated transfection, non-chemical methods such as electroporation, particle bombardment, or microinjection, and infection with a virus that contains the nucleic acid molecule of interest (sometimes termed “transduction”). Markers can be used for the identification and/or selection of cells that have taken up the vector and, typically, express the nucleic acid. Cells can be cultured in appropriate media to select such cells and, optionally, establish a stable cell line.

As used herein, the term “vaccine” relates to a pharmaceutical preparation (pharmaceutical composition) or product that upon administration induces an immune response, in particular a cellular immune response, which recognizes and attacks a pathogen or a diseased cell such (e.g., cancer cell). A vaccine may be used for the prevention or treatment of a disease. The term “individualized cancer vaccine” concerns a particular cancer patient and means that a cancer vaccine is adapted to the needs or special circumstances of an individual cancer patient.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

### Genes of the Clustered Protocadherin Locus

The present invention relates to genes of the clustered protocadherin locus (CPL). The locus is comprised of three clusters of genes designated α, β, and γ. The α cluster includes the isoforms PCDHA1, PCDHA2, PCDHA3, PCDHA4, PCDHA5, PCDHA6, PCDHA7, PCDHA8, PCDHA9, PCDHA10, PCDHA11, PCDHA12, PCDHA13, PCDHAC1, and PCDHAC2. The β cluster includes the genes PCDHB1, PCDHB2, PCDHB3, PCDHB4, PCDHB5, PCDHB6, PCDHB7, PCDHB8, PCDHB9, PCDHB10, PCDHB11, PCDHB12, PCDHB13, PCDHB14, PCDHB15, PCDHB17P, and PCDHB18P. The γ cluster includes the CPL isoforms PCDHGA1, PCDHGA2, PCDHGA3, PCDHGA4, PCDHGA5, PCDHGA6, PCDHGA7, PCDHGA8, PCDHGA9, PCDHGA10, PCDHGA11, PCDHGA12, PCDHGB1, PCDHGB2, PCDHGB3, PCDHGB4, PCDHGB5, PCDHGB6, PCDHGB7, PCDHGB8P, PCDHGC3, PCDHGC4, and PCDHGC5. We previously disclosed that certain members of the CPL were differentially-expressed in embryonic (pre-fetal) cells of most somatic cell types and other members were expressed in the fetal and adult stages of development in a similarly unexpectedly large number of somatic cell types (see PCT Patent Application Ser. Nos. PCT/US2014/040601 and PCT/US2017/036452, each of which is incorporated by reference herein in its entirety). We therefore propose herein that the CPL isoforms play a critical role is specific cell-cell adhesion and organogenesis during the embryonic stages of development, that the precise combination of isoforms depend on the particular differentiated cell type, and that the transition to a fetal and adult pattern of expression reflects a post-embryonic inhibition of regenerative potential in that tissue. We furthermore teach that cancers of a similarly diverse and surprisingly large number of cancer types revert to an embryonic pattern of CPL expression, but that said cancer cells are biphasic and can shift between an adult and an embryonic pattern of CPL isoform expression. Furthermore, the present invention teaches that the resulting heterogeneity leads to a spectrum of characteristics within tumors. The embryonic pattern of CPL isoform expression leads to cell-cell aggregation, and is associated with rapid proliferation, increased aerobic glycolysis, and sensitivity to apoptosis such as when exposed to radio- or chemotherapy. The adult pattern of CPL isoform expression leads to a loss of cell-cell aggregation and instead an epithelial-mesenchymal transformation, is associated with slower rates of proliferation, increased oxidative phosphorylation, and relative insensitivity to apoptosis such as when exposed to radio- or chemotherapy. The latter cells are often referred to as cancer cells that have undergone epithelial-mesenchymal transition (EMT) or cancer stem cells (CSCs). Therefore, the present invention teaches the contrary doctrine that CSCs are not more undifferentiated than other cancer cells, but quite the opposite, they are more mature and adult-like. Further, the present invention provides that the transition of the adult status of CPL isoform expression to embryonic expression may occur early in the pathogenesis of cancer. By way of non-limiting example, embryonic isoform expression may occur in intestinal adenomas before the progression to adenocarcinomas occurs. This provides a means of detecting early stages of oncogenesis in all the diverse cell and cancer types disclosed herein as well as means of targeting said cells for therapeutic effect.

In addition, we disclose that the relative high ratio of expression of the lamin B1 (LMNB1) gene compared to lamin A/C (LMNA) organizes chromatin in the CPL leading to hypermethylation of the CPL isoform CGIs, and an embryonic pattern of CPL isoform expression which is characterized by activation of members of the α and β clusters and repression of members of the γ locus with the exception of PCDHGB4 and PCDHGB6 which are relatively highly expressed in embryonic cells. In contrast, the relative high ratio of expression of the gene LMNA compared to LMNB1 organizes chromatin in the CPL leading to decreased methylation of the CPL isoform CGIs, and an adult pattern of CPL isoform expression which is characterized by inhibition of expression of members of the α and β clusters and increased expression of members of the γ locus with the exception of PCDHGB4 and PCDHGB6 which are relatively highly expressed in embryonic cells.

Up-regulation of the α and β CPL isoforms, PCDHGB4, and PCDHGB6 and downregulation of the γ isoforms (with the exception of PCDHGB4 and PCDHGB6) may be achieved by the up-regulation of the LMNB1/LMNA expression ratio. This, in turn, may be effectively accomplished by the exogenously induced expression of LMNB1 with or without the inhibition of expression of LMNA or the inhibition of LMNA with or without the induced expression of LMNB1. This altered gene expression may be accomplished by RNA or DNA-mediated induction of expression or siRNA using the methods described herein.

In some embodiments, expression of LMNA and/or LMNB1 is inhibited by introducing exogenous nucleotides RNA and/or DNA into the cell by transfection. Transfection may be performed using Lipfectamine or equivalent lipid transfection reagent. After introduction of exogenous nucleotides (e.g., RNA and/or DNA) into the cell, RNA, such as siRNA, inhibits expression of the target gene as described herein. Other methods to alter gene expression, can include Clustered Regularly Interspaced Short Palindromic Repeats(CRISPR)/Cas9 directed to the target gene (e.g., LMNA/LMNB1). For example, a tracrRNA and crRNA or a single guide RNA (sgRNA) is constructed to target Cas9 to the gene of interest (e.g., LMNA/LMNB1). Cas9 or a dead-Cas9 enzyme can inhibit expression from the target gene. Cas9 and the guide RNA can be introduced into a cell by transduction of expression constructs into the cell.

In addition to regulating the regenerative and cancer phenotypes of cells, some members to the gamma family of CPL isoforms play a role in cellular aging and the aging of tissues in vivo. For example, we disclose herein that the gene PCDHGC3 is up-regulated in adult cells compared to embryonic (pre-fetal) cells and is further up-regulated 3-5-fold in cells cultured to senescence in vitro. The promoter of the PCDHGC3 gene is relatively methylated in embryonic and cancer cells compared to adult cells and further demethylated in senescent cells. Therefore down-regulating the expression of the gene or introducing RNAi to cells and tissues is capable of inducing tissue regeneration in adult tissues. Furthermore, up-regulating the expression of PCDHGC3 or introducing the RNA or protein encoded by the PCDHGC3 gene to cancer cells can induce cancer maturation and decrease the rate of proliferation of cancer cells.

Therefore these unanticipated results provide novel diagnostic and therapeutic compositions and methods as described below.

### Diagnostic Applications

The identification of altered expression of CPL isoforms in cancer can be utilized in diagnosis and the detection of cancer cells in vivo for targeted removal. In the case of diagnosis, the presence of embryonic CPL isoform expression in a fetus or adult indicates the likelihood of cells progressing towards malignancy or outright malignant cells that are relatively rapidly proliferating and sensitive to radio- or chemotherapy. In contrast, the detection of cancer cells expressing a fetal or adult pattern of CPL isoform expression identifies cells that have undergone EMT, are relatively resistant to radio- or chemotherapy, and are prone to metastasis. Detection of the embryonic vs fetal-adult state of cells can be accomplished through the detection of embryonic hypermethylated DMRs (see PCT Patent Application Ser. No. PCT/US2020/047707, which is incorporated by reference herein in its entirety), or by detecting transcribed RNA for embryonic CPL isoforms as described herein, or by detecting the CPL isoform extracellular antigens such as through Fluorescence-activated Cell Sorting (FACS), biotin-labeled detection antibodies, or equivalent methods to detect cell surface antigens.

Reagents that are capable of detecting embryonic CPL isoform patterns of expression safely in vivo are useful in detecting cancer in real-time wherein a ligand is introduced to the tissue through the circulation, local injection, or topical application wherein said ligand can directly emit light such as with fluorescence allowing a surgeon to precisely demarcate the location of precancerous or cancerous cells for destruction or removal. Methods described in U.S. Pat. No. 9,451,882, which is incorporated by reference herein in its entirety.

### Therapeutic Applications

The teaching of the present invention, in particular, the novel insight that diverse somatic embryonic cells outside of the central nervous system express an embryonic pattern of CPL isoforms, and also the insight that cancer cells of diverse cell types have frequently reverted to an embryonic pattern of CPL isoform expression but said cancer cells can revert to an adult pattern of CPL isoform expression in what is commonly called epithelial-mesenchymal transition (also inappropriately called “cancer stem cells”) allow numerous therapeutic strategies.

Induced Tissue Regeneration (iTR) and Induced Senolysis of Cancer Stem Cells (iS-CSC) Using CPL Isoforms

We previously disclosed the use of PCDHB2 and PCDHB17 (see PCT Patent Application Ser. No. PCT/US2014/040601 is incorporated by reference herein in its entirety) and PCDHA2, PCDHA4, PCDHA5, PCDHA10, PCDHA11, PCDHAC1, PCDHB2, PCDHB5, PCDHB9, PCDHB10, PCDHB14, PCDHB16, PCDHGB3, PCDHGB4, PCDHGB6, PCDHGA6, PCDHGA7, PCDHGA9, PCDHGA10, and PCDHGA12 (see PCT Patent Application Ser. No. PCT/US2017/036452 is incorporated by reference herein in its entirety) for inducing tissue regeneration (iTR) and induced cancer maturation (iCM), herein we provide improved methods of reprogramming diverse adult cell types to a state that promotes scarless regeneration (iTR) or induced senolysis of cancer stem cells (iS-CSC) by means of targeting CPL isoforms.

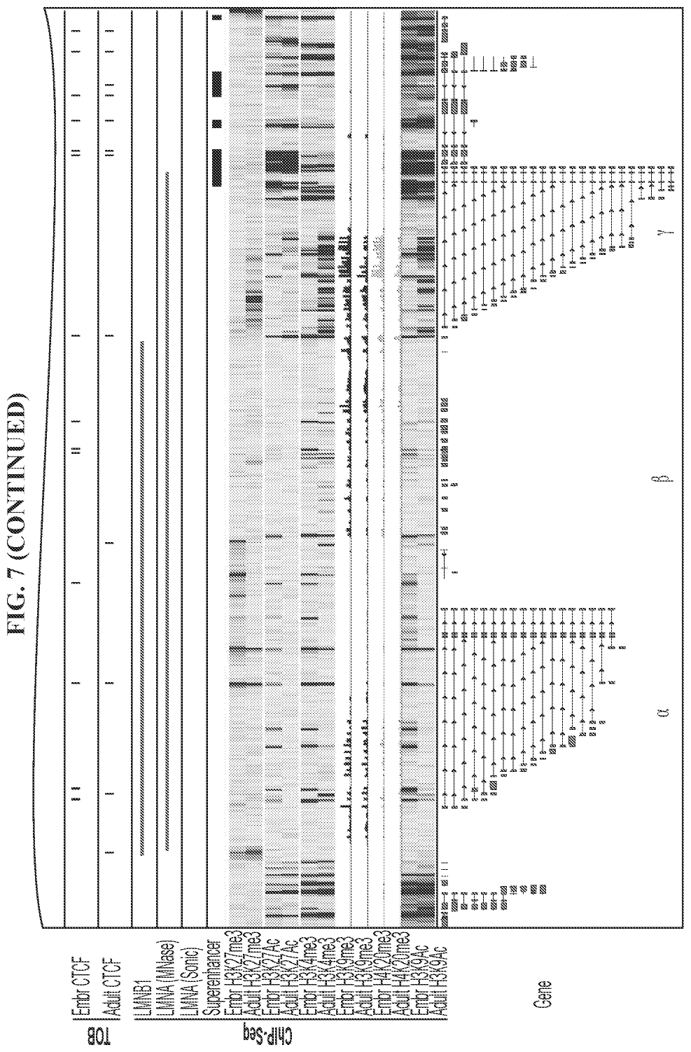

As shown in FIG. 7, the CPL locus is unexpectedly enclosed in unusually tightly-controlled chromatin reflecting an association with lamin B1 in embryonic cells, and an increased amount of lamin A in fetal and adult cells of various somatic cell types. The region has very high relative levels of H3K9me3 and H4K20me3 histone modification characteristic of heterochromatin such as in peri-centromeric or peri-telomeric DNA. Reprogramming factors, even pioneer factors such as those encoded by the genes SOX2, OCT4, KLF4, and NANOG, therefore inefficiently reprogram embryonic CPL isoform expression in adult cells. Therefore, the improved methods of iTR and/or iS-SCS utilizes two steps that can be performed simultaneously or separated in time, preferably step one occurring first. In the first step, H3K9me3 heterochromatin is relaxed through the inhibition of one or more of the methytransferases responsible for H3K9me3 methylation; namely, those encoded by the genes SUV39H1, SUV39H2, and SETDB1. Reduction in the activity of these gene products can be readily achieved by methods known in the art such as the use of siRNA targeting SUV39H1 transcript, or preferably SUV39H1, SUV39H2, and SETDB1 transcripts. Alternatively, or in addition small molecule inhibitors of the enzymes can be used such as the SUV39H1 inhibitor Chaetocin, the SUV39H2 inhibitor OTS186935 hydrochloride, and the SETDB1 inhibitors mithramycin A, demycarosyl-3D-β-D-digitoxosyl-mithramycin SK (DIG-MSK), also known as “EC-8042”, streptonigrin and emetine.

In the second step, the target normal adult or the cancer cells with adult patterns of CPL isoform expression are transiently exposed to factors that in other conditions are capable of reprogramming the cells to pluripotency. The factors may include constructs that introduce RNA into cells either directly or through gene expression vectors that are capable of inducing pluripotency if allowed to react with cells for a sufficient period of time, but for lesser times can cause iTR. Gene expression vectors are known in the art including, but not limited to, linear polynucleotides, polynucleotides associated with ionic or amphiphilic compounds, plasmids, and viral vectors. Methods of introducing gene expression vectors into a cell are known in the art. For example, transfection using Lipofectamine or equivalent reagents can be used. Preferably, the RNAs do not include all of the RNAs needed for reprogramming to pluripotency and instead include only LIN28A or LIN28B optionally together with an agent to increase telomere length such as RNA for the catalytic component of telomerase (TERT). Most preferably, the agents to induce iTR are genes/factors induced by LIN28A or LIN28B-encoded proteins such as GFER, optionally in combination with an agent that increases telomere length such as the RNA or gene encoding TERT, and/or in combination with the factors disclosed herein important for iTR such as 0.05-5 mM valproic acid, preferably 0.5 mM valproic acid, 1-100 ng/mL AMH, preferably 10 ng/mL AMH, and 2-200 ng/mL GFER, preferably 20 ng/mL. When administered in vivo, such factors are preferably administered in a slow-release hydrogel matrix such as one comprised of chemically modified and crosslinked hyaluronic acid and collagen such as HyStem matrices.

More preferably, factors are chosen from agents capable in other conditions of inducing pluripotency in somatic cell types. Such agents include the following compounds individually or in combination: the genes OCT4, SOX2, KLF4, NANOG, ESRRB, NR5A2, CEBPA, MYC, TERT, LIN28A and LIN28B alone and in combination. Nonlimiting examples are the transient expression by AAV vectors transiently expressing from 1-2 weeks the combination of factors LIN28B, OCT4, SOX2, NANOG, and TERT, or alternatively, KLF4, OCT4, SOX2, and TERT at levels comparable to that in normal hES cells. Said factors may also include small molecule compounds such as combinations of the following compounds: inhibitors of glycogen synthase 3 (GSK3) including but not limited to CHIR99021; inhibitors of TGF-beta signaling including but not limited to SB431542, A-83-01, and E616452; HDAC inhibitors including but not limited to aliphatic acid compounds including but not limited to: valproic acid, phenylbutyrate, and n-butyrate; cyclic tetrapeptides including trapoxin B and the depsipeptides; hydroxamic acids such as trichostatin A, vorinostat (SAHA), belinostat (PXD101), LAQ824, panobinostat (LBH589), and the benzamides entinostat (MS-275), CI994, mocetinostat (MGCD0103); those specifically targeting Class I (HDAC1, HDAC2, HDAC3, and HDAC8), IIA (HDAC4, HDAC5, HDAC7, and HDAC9), IIB (HDAC6 and HDAC10), III (SIRT1, SIRT2, SIRT3, SIRT4, SIRT5, SIRT6, or SIRT7) including the sirtuin inhibitors nicotinomide, diverse derivatives of NAD, dihydrocoumarin, naphthopyranone, and 2-hydroxynaphthaldehydes, or IV (HDAC11) deacetylases; inhibitors of H3K4/9 histone demethylase LSD1 including but not limited to parnate; inhibitors of Dot1L including but not limited to EPZ004777; inhibitors of G9a including but not limited to Bix01294; inhibitors of EZH2 including but not limited to DZNep, inhibitors of DNA methyltransferase including but not limited to RG108; 5-aza-2′deoxycyidine (trade name Vidaza and Azadine); vitamin C which can inhibit DNA methylation, increase Tet1 which increases 5hmC which is a first step of demethylation; activators of 3′ phosphoinositide-dependent kinase 1 including but not limited to PS48; promoters of glycolysis including but not limited to Quercetin and fructose 2, 6-bisphosphate (an activator of phosphofructokinase 1); agents that promote the activity of the HIF1 transcription complex including but not limited to Quercetin; RAR agonists including but not limited to AM580, CD437, and TTNPB; agents that mimic hypoxia including but not limited to Resveratrol; agents that increase telomerase activity including but not limited to the exogenous expression of the catalytic component of telomerase (TERT), agents that promote epigenetic modifications via downregulation of LSD1, a H3K4-specific histone demethylase including but not limited to lithium; or inhibitors of the MAPK/ERK pathway including but not limited to PD032590. Such compounds may be administered in diverse combinations, concentrations, and for differing periods of time, to optimize the effect of iTR on cells cultured in vitro using markers of global iTR such as by assaying for decreased expression of COX7A1 or NAALADL1, or other inhibitors of iTR as described herein, and/or assaying for increased expression of PCDHB2 or AMH or other activators or iTR as described herein, or in injured or diseased tissues in vivo, or in modulating the lifespan of animals in vivo.

### Screens, Reporter Molecules, Cells, and Membranes

In general, detectable moieties useful in the reporter molecules of the invention include light-emitting or light-absorbing compounds that generate or quench a detectable fluorescent, chemiluminescent, or bioluminescent signal. In some embodiments, activation of CPL isoform genes or inhibition of other isoform genes causes release of the detectable moiety into a liquid medium, and the signal generated or quenched by the released detectable moiety present in the medium (or a sample thereof) is detected. In some embodiments, the resulting signal causes an alteration in a property of the detectable moiety, and such alteration can be detected, e.g., as an optical signal. For example, the signal may alter the emission or absorption of electromagnetic radiation (e.g., radiation having a wavelength within the infrared, visible or UV portion of the spectrum) by the detectable moiety. In some embodiments, a reporter molecule comprises a fluorescent or luminescent moiety, and a second molecule serves as quencher that quenches the fluorescent or luminescent moiety. Such alteration can be detected using apparatus and methods known in the art.

In many embodiments of the invention, the reporter molecule is a genetically encodable molecule that can be expressed by a cell, and the detectable moiety comprises, e.g., a detectable polypeptide. Thus in some embodiments, the reporter molecule is a polypeptide comprising a fluorescent polypeptides such as green, blue, sapphire, yellow, red, orange, and cyan fluorescent proteins and derivatives thereof (e.g., enhanced GFP); monomeric red fluorescent protein and derivatives such as those known as “mFruits”, e.g., mCherry, mStrawberry, mTomato, etc., and luminescent proteins such as aequorin. (It will be understood that in some embodiments, the fluorescence or luminescence occurs in the presence of one or more additional molecules, e.g., an ion such as a calcium ion and/or a prosthetic group such as coelenterazine.) In some embodiments, the detectable moiety comprises an enzyme that acts on a substrate to produce a fluorescent, luminescent, colored, or otherwise detectable product. Examples of enzymes that may serve as detectable moieties include luciferase; beta-galactosidase; horseradish peroxidase; alkaline phosphatase; etc. (It will be appreciated that the enzyme is detected by detecting the product of the reaction.) In some embodiments, the detectable moiety comprises a polypeptide tag that can be readily detected using a second agent such as a labeled (e.g., fluorescently labeled) antibody. For example, fluorescently labeled antibodies that bind to the HA, Myc, or a variety of other peptide tags are available. Thus the invention encompasses embodiments in which a detectable moiety can be detected directly (i.e., it generates a detectable signal without requiring interaction with a second agent) and embodiments in which a detectable moiety interacts (e.g., binds and/or reacts) with a second agent and such interaction renders the detectable moiety detectable, e.g., by resulting in generation of a detectable signal or because the second agent is directly detectable. In embodiments in which a detectable moiety interacts with a second agent to produce a detectable signal, the detectable moiety may react with the second agent is acted on by a second agent to produce a detectable signal. In many embodiments, the intensity of the signal provides an indication of the amount of detectable moiety present. e.g., in a sample being assessed or in area being imaged. In some embodiments, the amount of detectable moiety is optionally quantified, e.g., on a relative or absolute basis, based on the signal intensity.

The invention provides nucleic acids comprising a sequence that encodes a reporter polypeptide of the invention. In some embodiments, a nucleic acid encodes a precursor polypeptide of a reporter polypeptide of the invention. In some embodiments, the sequence encoding the polypeptide is operably linked to expression control elements (e.g., a promoter or promoter/enhancer sequence) appropriate to direct transcription of mRNA encoding the polypeptide. The invention further provides expression vectors comprising the nucleic acids. Selection of appropriate expression control elements may be based, e.g., on the cell type and species in which the nucleic acid is to be expressed. One of ordinary skill in the art can readily select appropriate expression control elements and/or expression vectors. In some embodiments, expression control element(s) are regulatable, e.g., inducible or repressible. Exemplary promoters suitable for use in bacterial cells include, e.g., Lac, Trp, Tac, araBAD (e.g., in a pBAD vectors), phage promoters such as T7 or T3. Exemplary expression control sequences useful for directing expression in mammalian cells include, e.g., the early and late promoters of SV40, adenovirus or cytomegalovirus immediate early promoter, or viral promoter/enhancer sequences, retroviral LTRs, promoters or promoter/enhancers from mammalian genes, e.g., actin, EF-1 alpha, phosphoglycerate kinase, etc. Regulatable (e.g., inducible or repressible) expression systems such as the Tet-On and Tet-Off systems (regulatable by tetracycline and analogs such as doxycycline) and others that can be regulated by small molecules such as hormones receptor ligands (e.g., steroid receptor ligands, which may or may not be steroids), metal-regulated systems (e.g., metallothionein promoter), etc.

The invention further provides cells and cell lines that comprise such nucleic acids and/or vectors. In some embodiments, the cells are eukaryotic cells, e.g., fungal, plant, or animal cells. In some embodiments, the cell is a vertebrate cell, e.g., a mammalian cell, e.g., a human cell, non-human primate cell, or rodent cell. Often a cell is a member of a cell line, e.g., an established or immortalized cell line that has acquired the ability to proliferate indefinitely in culture (e.g., as a result of mutation or genetic manipulation such as the constitutive expression of the catalytic component of telomerase). Numerous cell lines are known in the art and can be used in the instant invention. Mammalian cell lines include, e.g., HEK-293 (e.g., HEK-293T), CHO, NIH-3T3, COS, and HeLa cell lines. In some embodiments, a cell line is a tumor cell line. In other embodiments, a cell is non-tumorigenic and/or is not derived from a tumor. In some embodiments, the cells are adherent cells. In some embodiments, non-adherent cells are used. In some embodiments, a cell is of a cell type or cell line is used that has been shown to naturally have a subset of iTR reprogramming genes expressed or iTR inhibitor genes not expressed. If a cell lacks one or more TR activator or inhibitor genes, the cell can be genetically engineered to express such protein(s). In some embodiments, a cell line of the invention is descended from a single cell. For example, a population of cells can be transfected with a nucleic acid encoding the reporter polypeptide and a colony derived from a single cell can be selected and expanded in culture. In some embodiments, cells are transiently transfected with an expression vector that encodes the reporter molecule. Cells can be co-transfected with a control plasmid, optionally expressing a different detectable polypeptide, to control for transfection efficiency (e.g., across multiple runs of an assay).

Uses of iTR, iS-CSC, and ICM Factors

### Pharmaceutical Compositions

iTR, iS-CSC, and iCM factors have a variety of different uses. Non-limiting examples of such uses are discussed herein. In some embodiments, an iTR factor is used to enhance regeneration of an organ or tissue. In some embodiments, an iTR factor is used to enhance regeneration of a limb, digit, cartilage, heart, blood vessel, bone, esophagus, stomach, liver, gallbladder, pancreas, intestines, rectum, anus, endocrine gland (e.g., thyroid, parathyroid, adrenal, endocrine portion of pancreas), skin, hair follicle, thymus, spleen, skeletal muscle, focal damaged cardiac muscle, smooth muscle, brain, spinal cord, peripheral nerve, ovary, fallopian tube, uterus, vagina, mammary gland, testes, vas deferens, seminal vesicle, prostate, penis, pharynx, larynx, trachea, bronchi, lungs, kidney, ureter, bladder, urethra, eye (e.g., retina, cornea), or ear (e.g., organ of Corti). In some embodiments, an iTR factor is used to enhance regeneration of a stromal layer, e.g., a connective tissue supporting the parenchyma of a tissue. In some embodiments, an iTR factor is used to enhance regeneration following surgery, e.g., surgery that entails removal of at least a portion of a diseased or damaged tissue, organ, or other structure such as a limb, digit, etc. For example, such surgery might remove at least a portion of a liver, lung, kidney, stomach, pancreas, intestine, mammary gland, ovary, testis, bone, limb, digit, muscle, skin, etc. In some embodiments, the surgery is to remove a tumor. In some embodiments, an iTR factor is used to promote scarless regeneration of skin following trauma, surgery, disease, and burns.

Enhancing regeneration can include any one or more of the following, in various embodiments: (a) increasing the rate of regeneration; (b) increasing the extent of regeneration; (c) promoting establishment of appropriate structure (e.g., shape, pattern, tissue architecture, tissue polarity) in a regenerating tissue or organ or other body structure; (d) promoting growth of new tissue in a manner that retains and/or restores function. While use of an iTR factor to enhance regeneration is of particular interest, the invention encompasses use of an iTR factor to enhance repair or wound healing in general, without necessarily producing a detectable enhancement of regeneration. Thus, the invention provides methods of enhancing repair or wound healing, wherein an iTR factor is administered to a subject in need thereof according to any of the methods described herein.

Numerous aspects of aging and age-related disease are taught in the present invention to addressable with iTR therapy. These manifestations of aging include age-related vascular dysfunction including peripheral vascular, coronary, and cerebrovascular disease; musculoskeletal disorders including osteoarthritis, intervertebral disc degeneration, bone fractures, tendon and ligament tears, and limb regeneration; neurological disorders including stroke and spinal cord injuries; muscular disorders including muscular dystrophy, sarcopenia, myocardial infarction, and heart failure; endocrine disorders including Type I diabetes, Addison's disease, hypothyroidism, and pituitary insufficiency; digestive disorders including pancreatic exocrine insufficiency; ocular disorders including macular degeneration, retinitis pigmentosa, and neural retinal degeneration disorders; dermatological conditions including skin burns, lacerations, surgical incisions, alopecia, graying of hair, and skin aging; pulmonary disorders including emphysema and interstitial fibrosis of the lung; auditory disorders including hearing loss; and hematological disorders such as aplastic anemia and failed hematopoietic stem cell grafts.

In some embodiments, the invention provides a method of enhancing regeneration in a subject in need thereof, the method comprising administering an effective amount of an iTR factor to the subject. In some embodiments, an effective amount of a compound (e.g., an iTR factor) is an amount that results in an increased rate or extent of regeneration of damaged tissue as compared with a reference value (e.g., a suitable control value). In some embodiments, the reference value is the expected (e.g., average or typical) rate or extent of regeneration in the absence of the compound (optionally with administration of a placebo). In some embodiments, an effective amount of an iTR factor is an amount that results in an improved structural and/or functional outcome as compared with the expected (e.g., average or typical) structural or functional outcome in the absence of the compound. In some embodiments, an effective amount of a compound, e.g., an iTR factor, results in enhanced blastema formation and/or reduced scarring. Extent or rate of regeneration can be assessed based on dimension(s) or volume of regenerated tissue, for example. Structural and/or functional outcome can be assessed based on, e.g., visual examination (optionally including use of microscopy or imaging techniques such as X-rays, CT scans, MRI scans, PET scans) and/or by evaluating the ability of the tissue, organ, or other body part to perform one or more physiological processes or task(s) normally performed by such tissue, organ, or body part. Typically, an improved structural outcome is one that more closely resembles normal structure (e.g., structure that existed prior to tissue damage or structure as it exists in a normal, healthy individual) as compared with the structural outcome that would be expected (e.g., average or typical outcome) in the absence of treatment with an iTR factor. One of ordinary skill in the art can select an appropriate assay or test for function. In some embodiments, an increase in the rate or extent of regeneration as compared with a control value is statistically significant (e.g., with a p value of <0.05, or with a p value of <0.01) and/or clinically significant. In some embodiments, an improvement in structural and/or functional outcome as compared with a control value is statistically significant and/or clinically significant. “Clinically significant improvement” refers to an improvement that, within the sound judgement of a medical or surgical practitioner, confers a meaningful benefit on a subject (e.g., a benefit sufficient to make the treatment worthwhile). It will be appreciated that in many embodiments an iTR modulator, e.g., an iTR factor, administered to a subject of a particular species (e.g., for therapeutic purposes) is a compound that modulates, e.g., inhibits, the endogenous TR genes expressed in subjects of that species. For example, if a subject is human, a compound that inhibits the activity of human TR inhibitor gene products and activates the activity of human TR activator gene products would typically be administered.

In some embodiments, the iTR factor is used to enhance skin regeneration, e.g., after a burn (thermal or chemical), scrape injury, or other situations involving skin loss, e.g., infections such as necrotizing fasciitis or purpura fulminans. In some embodiments, a burn is a second or third degree burn. In some embodiments a region of skin loss has an area of at least 10 cm². In one aspect, an iTR factor enhances regeneration of grafted skin. In one aspect, an iTR factor reduces excessive and/or pathological wound contraction or scarring.

In some embodiments, an iTR factor is used to enhance bone regeneration, e.g., in a situation such as non-union fracture, implant fixation, periodontal or alveolar ridge augmentation, craniofacial surgery, or other conditions in which generation of new bone is considered appropriate. In some embodiments, an iTR factor is applied to a site where bone regeneration is desired. In some embodiments, an iTR factor is incorporated into or used in combination with a bone graft material. Bone graft materials include a variety of ceramic and proteinaceous materials. Bone graft materials include autologous bone (e.g., bone harvested from the iliac crest, fibula, ribs, etc.), allogeneic bone from cadavers, and xenogeneic bone. Synthetic bone graft materials include a variety of ceramics such as calcium phosphates (e.g. hydroxyapatite and tricalcium phosphate), bioglass, and calcium sulphate, and proteinaceous materials such as demineralized bone matrix (DBM). DBM can be prepared by grinding cortical bone tissues (generally to 100-500 μm sieved particle size), then treating the ground tissues with hydrochloric acid (generally 0.5 to 1 N). In some embodiments, an iTR factor is administered to a subject together with one or more bone graft materials. The iTR factor may be combined with the bone graft material (in a composition comprising an iTR factor and a bone graft material) or administered separately, e.g., after placement of the graft. In some embodiments, the invention provides a bone paste comprising an iTR factor. Bone pastes are products that have a suitable consistency and composition such that they can be introduced into bone defects, such as voids, gaps, cavities, cracks etc., and used to patch or fill such defects, or applied to existing bony structures. Bone pastes typically have sufficient malleability to permit them to be manipulated and molded by the user into various shapes. The desired outcome of such treatments is that bone formation will occur to replace the paste, e.g., retaining the shape in which the paste was applied. The bone paste provides a supporting structure for new bone formation and may contain substance(s) that promote bone formation. Bone pastes often contain one or more components that impart a paste or putty-like consistency to the material, e.g., hyaluronic acid, chitosan, starch components such as amylopectin, in addition to one or more of the ceramic or proteinaceous bone graft materials (e.g., DBM, hydroxyapatite) mentioned above.

In some embodiments, an iTR factor enhances the formation and/or recruitment of osteoprogenitor cells from undifferentiated mesechymal cells and/or enhances the differentiation of osteoprogenitor cells into cells that form new bone (osteoblasts).

In some embodiments, an iTR factor is administered to a subject with osteopenia or osteoporosis, e.g., to enhance bone regeneration in the subject.

In some embodiments, an iTR factor is used to enhance regeneration of a joint (e.g., a fibrous, cartilaginous, or synovial joint). In some embodiments, the joint is an intervertebral disc. In some embodiments, a joint is a hip, knee, elbow, or shoulder joint. In some embodiments, an iTR factor is used to enhance regeneration of dental and/or periodontal tissues or structures (e.g., pulp, periodontal ligament, teeth, periodontal bone).

In some embodiments, an iTR factor is used to reduce glial scarring in CNS and PNS injuries.

In some embodiments, an iTR factor is used to reduce adhesions and stricture formation in internal surgery.

In some embodiments, an iTR factor is used to decrease scarring in tendon and ligament repair improving mobility.

In some embodiments, an iTR factor is used to reduce vision loss following eye injury.

In some embodiments, an iTR factor is administered to a subject in combination with cells. The iTR factor and the cells may be administered separately or in the same composition. If administered separately, they may be administered at the same or different locations. The cells can be autologous, allogeneic, or xenogeneic in various embodiments. The cells can comprise progenitor cells or stem cells, e.g., adult stem cells. As used herein, a stem cell is a cell that possesses at least the following properties: (i) self-renewal, i.e., the ability to go through numerous cycles of cell division while still maintaining an undifferentiated state; and (ii) multipotency or multidifferentiative potential, i.e., the ability to generate progeny of several distinct cell types (e.g., many, most, or all of the distinct cell types of a particular tissue or organ). An adult stem cell is a stem cell originating from non-embryonic tissues (e.g., fetal, post-natal, or adult tissues). As used herein, the term “progenitor cell” encompasses cells multipotent and cells that are more differentiated than pluripotent stem cells but not fully differentiated. Such more differentiated cells (which may arise from embryonic progenitor cells) have reduced capacity for self-renewal as compared with embryonic progenitor cells. In some embodiments, an iTR factor is administered in combination with mesenchymal progenitor cells, neural progenitor cells, endothelial progenitor cells, hair follicle progenitor cells, neural crest progenitor cells, mammary stem cells, lung progenitor cells (e.g., bronchioalveolar stem cells), muscle progenitor cells (e.g., satellite cells), adipose-derived progenitor cells, epithelial progenitor cells (e.g., keratinocyte stem cells), and/or hematopoietic progenitor cells (e.g., hematopoietic stem cells). In some embodiments, the cells comprise induced pluripotent stem cells (iPS cells), or cells that have been at least partly differentiated from iPS cells. In some embodiments, the progenitor cells comprise adult stem cells. In some embodiments, at least some of the cells are differentiated cells, e.g., chondrocytes, osteoblasts, keratinocytes, hepatocytes. In some embodiments, the cells comprise myoblasts.

In some embodiments, an iTR factor is administered in a composition (e.g., a solution) comprising one or more compounds that polymerizes or becomes cross-linked or undergoes a phase transition in situ following administration to a subject, typically forming a hydrogel. The composition may comprise monomers, polymers, initiating agents, crosslinking agents, etc. The composition may be applied (e.g., using a syringe) to an area where regeneration is needed, where it forms a gel in situ, from which an iTR factor is released over time. Gelation may be triggered, e.g., by contact with ions in body fluids or by change in temperature or pH, or by light, or by combining reactive precursors (e.g., using a multi-barreled syringe). (See, e.g., U.S. Pat. No. 6,129,761; Yu L, Ding J. Injectable hydrogels as unique biomedical materials. Chem Soc Rev. 37(8):1473-81 (2008)). In some embodiments the hydrogel is a hyaluronic acid or hyaluronic acid and collagen I-containing hydrogel such as HyStem-C described herein. In some embodiments, the composition further comprises cells.

In some embodiments, an iTR factor is administered to a subject in combination with vectors expressing the catalytic component of telomerase. The vector may be administered separately or in the same composition. If administered separately, they may be administered at the same or different locations. The vector may express the telomerase catalytic component from the same species as the treated tissue or from another species. Said co-administration of the iTR factor with the telomerase catalytic component is particularly useful wherein the target tissue is from an aged individual and said individual is from the human species.

Other inventive methods comprise use of an iTR factor in the ex vivo production of living, functional tissues, organs, or cell-containing compositions to repair or replace a tissue or organ lost due to damage. For example, cells or tissues removed from an individual (either the future recipient, an individual of the same species, or an individual of a different species) may be cultured in vitro, optionally with an matrix, scaffold (e.g., a three dimensional scaffold) or mold (e.g., comprising a biocompatible, optionally biodegradable, material, e.g., a polymer such as HyStem-C), and their development into a functional tissue or organ can be promoted by contacting an iTR factor. The scaffold, matrix, or mold may be composed at least in part of naturally occurring proteins such as collagen, hyaluronic acid, or alginate (or chemically modified derivatives of any of these), or synthetic polymers or copolymers of lactic acid, caprolactone, glycolic acid, etc., or self-assembling peptides, or decellularized matrices derived from tissues such as heart valves, intestinal mucosa, blood vessels, and trachea. In some embodiments, the scaffold comprises a hydrogel. The scaffold may, in certain embodiments, be coated or impregnated with an iTR factor, which may diffuse out from the scaffold over time. After production ex vivo, the tissue or organ is grafted into or onto a subject. For example, the tissue or organ can be implanted or, in the case of certain tissues such as skin, placed on a body surface. The tissue or organ may continue to develop in vivo. In some embodiments, the tissue or organ to be produced at least in part ex vivo is a bladder, blood vessel, bone, fascia, liver, muscle, skin patch, etc. Suitable scaffolds may, for example, mimic the extracellular matrix (ECM). Optionally, an iTR factor is administered to the subject prior to, during, and/or following grafting of the ex vivo generated tissue or organ. In some aspects, a biocompatible material is a material that is substantially non-toxic to cells in vitro at the concentration used or, in the case of a material that is administered to a living subject, is substantially nontoxic to the subject's cells in the quantities and at the location used and does not elicit or cause a significant deleterious or untoward effect on the subject, e.g., an immunological or inflammatory reaction, unacceptable scar tissue formation, etc. It will be understood that certain biocompatible materials may elicit such adverse reactions in a small percentage of subjects, typically less than about 5%, 1%, 0.5%, or 0.1%.

In some embodiments, a matrix or scaffold coated or impregnated with an iTR factor or combinations of factors including those capable of causing a global pattern of iTR gene expression is implanted, optionally in combination with cells, into a subject in need of regeneration. The matrix or scaffold may be in the shape of a tissue or organ whose regeneration is desired. The cells may be stem cells of one or more type(s) that gives rise to such tissue or organ and/or of type(s) found in such tissue or organ.

In some embodiments, an iTR factor or combination of factors is administered directly to or near a site of tissue damage. “Directly to a site of tissue damage” encompasses injecting a compound or composition into a site of tissue damage or spreading, pouring, or otherwise directly contacting the site of tissue damage with the compound or composition. In some embodiments, administration is considered “near a site of tissue damage” if administration occurs within up to about 10 cm away from a visible or otherwise evident edge of a site of tissue damage or to a blood vessel (e.g., an artery) that is located at least in part within the damaged tissue or organ. Administration “near a site of tissue damage” is sometimes administration within a damaged organ, but at a location where damage is not evident. In some embodiments, following damage or loss of a tissue, organ, or other structure, an iTR factor is applied to the remaining portion of the tissue, organ, or other structure. In some embodiments, an iTR factor is applied to the end of a severed digit or limb) that remains attached to the body, to enhance regeneration of the portion that has been lost. In some embodiments, the severed portion is reattached surgically, and an iTR factor is applied to either or both faces of the wound. In some embodiments, an iTR factor is administered to enhance engraftment or healing or regeneration of a transplanted organ or portion thereof. In some embodiments, an iTR factor is used to enhance nerve regeneration. For example, an iTR factor may be infused into a severed nerve, e.g., near the proximal and/or distal stump. In some embodiments, an iTR factor is placed within an artificial nerve conduit, a tube composed of biological or synthetic materials within which the nerve ends and intervening gap are enclosed. The factor or factors may be formulated in a matrix to facilitate their controlled release over time. Said matrix may comprise a biocompatible, optionally biodegradable, material, e.g., a polymer such as that comprised of hyaluronic acid, including crosslinked hyaluronic acid or carboxymethyl hyaluronate crosslinked with PEGDA, or a mixture of carboxymethyl hyaluronate crosslinked by PEGDA with carboxymethyl-modified gelatin (HyStem-C).

In some embodiments the iTR factor is AgeX1547 described herein and may or may not be formulated for localization and slow release in carboxymethyl hyaluronate crosslinked by PEGDA with carboxymethyl-modified gelatin (HyStem-C) to induce iTR.

iTM and iCM factors such as exosomes derived from fetal or adult cells can be administered in physiological solutions such as saline, or slow-released in carboxymethyl hyaluronate crosslinked by PEGDA with carboxymethyl-modified gelatin (HyStem-C) to induce iTM or iCM.

In some embodiments, an iTR factor or combinations of factors is used to promote production of hair follicles and/or growth of hair. In some embodiments an iTR factor triggers regeneration of hair follicles from epithelial cells that do not normally form hair. In some embodiments, an iTR factor is used to treat hair loss, hair sparseness, partial or complete baldness in a male or female. In some embodiments, baldness is the state of having no or essentially no hair or lacking hair where it often grows, such as on the top, back, and/or sides of the head. In some embodiments, hair sparseness is the state of having less hair than normal or average or, in some embodiments, less hair than an individual had in the past or, in some embodiments, less hair than an individual considers desirable. In some embodiments, an iTR factor is used to promote growth of eyebrows or eyelashes. In some embodiments, an iTR factor is used to treat androgenic alopecia or “male pattern baldness” (which can affect males and females). In some embodiments, an iTR factor is used to treat alopecia areata, which involves patchy hair loss on the scalp, alopecia totalis, which involves the loss of all head hair, or alopecia universalis, which involves the loss of all hair from the head and the body. In some embodiments, an iTR factor is applied to a site where hair growth is desired, e.g., the scalp or eyebrow region. In some embodiments, an iTR factor is applied to or near the edge of the eyelid, to promote eyelash growth. In some embodiments, an iTR factor is applied in a liquid formulation. In some embodiments an iTR factor is applied in a cream, ointment, paste, or gel. In some embodiments, an iTR factor is used to enhance hair growth after a burn, surgery, chemotherapy, or other event causing loss of hair or hear-bearing skin.

In some embodiments, an iTR factor or combination of factors are administered to tissues afflicted with age-related degenerative changes to regenerate youthful function. Said age-related degenerative changes includes by way of nonlimiting example, age-related macular degeneration, coronary disease, osteoporosis, osteonecrosis, heart failure, emphysema, peripheral artery disease, vocal cord atrophy, hearing loss, Alzheimer's disease, Parkinson's disease, skin ulcers, and other age-related degenerative diseases. In some embodiments, said iTR factors are co-administered with a vector expressing the catalytic component of telomerase to extend cell lifespan.

In some embodiments, an iTR factor or factors are administered to enhance replacement of cells that have been lost or damaged due to insults such as chemotherapy, radiation, or toxins. In some embodiments such cells are stromal cells of solid organs and tissues.

Inventive methods of treatment can include a step of identifying or providing a subject suffering from or at risk of a disease or condition in which in which enhancing regeneration would be of benefit to the subject. In some embodiments, the subject has experienced injury (e.g., physical trauma) or damage to a tissue or organ. In some embodiments the damage is to a limb or digit. In some embodiments, a subject suffers from a disease affecting the cardiovascular, digestive, endocrine, musculoskeletal, gastrointestinal, hepatic, integumentary, nervous, respiratory, or urinary system. In some embodiments, tissue damage is to a tissue, organ, or structure such as cartilage, bone, heart, blood vessel, esophagus, stomach, liver, gallbladder, pancreas, intestines, rectum, anus, endocrine gland, skin, hair follicle, tooth, gum, lip, nose, mouth, thymus, spleen, skeletal muscle, smooth muscle, joint, brain, spinal cord, peripheral nerve, ovary, fallopian tube, uterus, vagina, mammary gland, testes, vas deferens, seminal vesicle, prostate, penis, pharynx, larynx, trachea, bronchi, lungs, kidney, ureter, bladder, urethra, eye (e.g., retina, cornea), or ear (e.g., organ of Corti).

In some embodiments a compound or composition is administered to a subject at least once within approximately 2, 4, 8, 12, 24, 48, 72, or 96 hours after a subject has suffered tissue damage (e.g., an injury or an acute disease-related event such as a myocardial infarction or stroke) and, optionally, at least once thereafter. In some embodiments a compound or composition is administered to a subject at least once within approximately 1-2 weeks, 2-6 weeks, or 6-12 weeks, after a subject has suffered tissue damage and, optionally, at least once thereafter.

In some embodiments of the invention, it may useful to stimulate or facilitate regeneration or de novo development of a missing or hypoplastic tissue, organ, or structure by, for example, removing the skin, removing at least some tissue at a site where regeneration or de novo development is desired, abrading a joint or bone surface where regeneration or de novo development is desired, and/or inflicting another type of wound on a subject. In the case of regeneration after tissue damage, it may be desirable to remove (e.g., by surgical excision or debridement) at least some of the damaged tissue. In some embodiments, an iTR factor is administered at or near the site of such removal or abrasion.

In some embodiments, an iTR factor is used to enhance generation of a tissue or organ in a subject in whom such tissue or organ is at least partially absent as a result of a congenital disorder, e.g., a genetic disease. Many congenital malformations result in hypoplasia or absence of a variety of tissues, organs, or body structures such as limbs or digits. In other instances a developmental disorder resulting in hypoplasia of a tissue, organ, or other body structure becomes evident after birth. In some embodiments, an iTR factor is administered to a subject suffering from hypoplasia or absence of a tissue, organ, or other body structure, in order to stimulate growth or development of such tissue, organ, or other body structure. In some aspects, the invention provides a method of enhancing generation of a tissue, organ, or other body structure in a subject suffering from hypoplasia or congenital absence of such tissue, organ, or other body structure, the method comprising administering an iTR factor to the subject. In some embodiments, an iTR factor is administered to the subject prior to birth, i.e., in utero. The various aspects and embodiments of the invention described herein with respect to regeneration are applicable to such de novo generation of a tissue, organ, or other body structure and are encompassed within the invention.

In some aspects, an iTR factor is used to enhance generation of tissue in any of a variety of situations in which new tissue growth is useful at locations where such tissue did not previously exist. For example, generating bone tissue between joints is frequently useful in the context of fusion of spinal or other joints.

iTR factors may be tested in a variety of animal models of regeneration. In one aspect, a modulator of iTR is tested in murine species. For example, mice can be wounded (e.g., by incision, amputation, transection, or removal of a tissue fragment). An iTR factor is applied to the site of the wound and/or to a removed tissue fragment and its effect on regeneration is assessed. The effect of a modulator of vertebrate TR can be tested in a variety of vertebrate models for tissue or organ regeneration. For example, fin regeneration can be assessed in zebrafish, e.g., as described in (Mathew L K, Unraveling tissue regeneration pathways using chemical genetics. J Biol Chem. 282(48):35202-10 (2007)), and can serve as a model for limb regeneration. Rodent, canine, equine, caprine, fish, amphibian, and other animal models useful for testing the effects of treatment on regeneration of tissues and organs such as heart, lung, limbs, skeletal muscle, bone, etc., are widely available. For example, various animal models for musculoskeletal regeneration are discussed in Tissue Eng Part B Rev. 16(1) (2010). A commonly used animal model for the study of liver regeneration involves surgical removal of a larger portion of the rodent liver. Other models for liver regeneration include acute or chronic liver injury or liver failure caused by toxins such as carbon tetrachloride. In some embodiments, a model for hair regeneration or healing of skin wounds involves excising a patch of skin, e.g., from a mouse. Regeneration of hair follicles, hair growth, re-epithelialization, gland formation, etc., can be assessed.

The compounds and compositions disclosed herein and/or identified using a method and/or assay system described herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or by inhalation, e.g., as an aerosol. The particular mode selected will depend, of course, upon the particular compound selected, the particular condition being treated and the dosage required for therapeutic efficacy. The methods of this invention, generally speaking, may be practiced using any mode of administration that is medically or veterinarily acceptable, meaning any mode that produces acceptable levels of efficacy without causing clinically unacceptable (e.g., medically or veterinarily unacceptable) adverse effects. Suitable preparations, e.g., substantially pure preparations, of one or more compound(s) may be combined with one or more pharmaceutically acceptable carriers or excipients, etc., to produce an appropriate pharmaceutical composition suitable for administration to a subject. Such pharmaceutically acceptable compositions are an aspect of the invention. The term “pharmaceutically acceptable carrier or excipient” refers to a carrier (which term encompasses carriers, media, diluents, solvents, vehicles, etc.) or excipient which does not significantly interfere with the biological activity or effectiveness of the active ingredient(s) of a composition and which is not excessively toxic to the host at the concentrations at which it is used or administered. Other pharmaceutically acceptable ingredients can be present in the composition as well. Suitable substances and their use for the formulation of pharmaceutically active compounds are well-known in the art (see, for example, “Remington's Pharmaceutical Sciences”, E. W. Martin, 19th Ed., 1995, Mack Publishing Co.: Easton, Pa., and more recent editions or versions thereof, such as Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types). Furthermore, compounds and compositions of the invention may be used in combination with any compound or composition used in the art for treatment of a particular disease or condition of interest.

A pharmaceutical composition is typically formulated to be compatible with its intended route of administration. For example, preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's. Examples of non-aqueous solvents are propylene glycol, polyethylene glycol, vegetable oils such as olive oil, and injectable organic esters such as ethyl oleate. fixed oils, polyethylene glycols, glycerine, propylene glycol or other synthetic solvents; preservatives, e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. pH can be adjusted with acids or bases, such as hydrochloric acid or sodium hydroxide. Such parenteral preparations can be enclosed in ampoules, disposable syringes or multiple dose vials made of glass or plastic.

For oral administration, compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like. Suitable excipients for oral dosage forms are, e.g., fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP).

For administration by inhalation, inventive compositions may be delivered in the form of an aerosol spray from a pressured container or dispenser which contains a suitable propellant, e.g., a gas such as carbon dioxide, a fluorocarbon, or a nebulizer. Liquid or dry aerosol (e.g., dry powders, large porous particles, etc.) can be used. The present invention also contemplates delivery of compositions using a nasal spray or other forms of nasal administration.

For topical applications, pharmaceutical compositions may be formulated in a suitable ointment, lotion, gel, or cream containing the active components suspended or dissolved in one or more pharmaceutically acceptable carriers suitable for use in such composition.

For local delivery to the eye, the pharmaceutically acceptable compositions may be formulated as solutions or micronized suspensions in isotonic, pH adjusted sterile saline, e.g., for use in eye drops, or in an ointment, or for intra-ocularly administration, e.g., by injection.

Pharmaceutical compositions may be formulated for transmucosal or transdermal delivery. For transmucosal or transdermal administration, penetrants appropriate to the barrier to be permeated may be used in the formulation. Such penetrants are generally known in the art. Inventive pharmaceutical compositions may be formulated as suppositories (e.g., with conventional suppository bases such as cocoa butter and other glycerides) or as retention enemas for rectal delivery.

In some embodiments, a composition includes one or more agents intended to protect the active agent(s) against rapid elimination from the body, such as a controlled release formulation, implants, microencapsulated delivery system, etc. Compositions may incorporate agents to improve stability (e.g., in the gastrointestinal tract or bloodstream) and/or to enhance absorption. Compounds may be encapsulated or incorporated into particles, e.g., microparticles or nanoparticles. Biodegradable, biocompatible polymers can be used, such as ethylene vinyl acetate, polyanhydrides, polyglycolic acid, PLGA, collagen, polyorthoesters, polyethers, and polylactic acid. Methods for preparation of such formulations will be apparent to those skilled in the art. For example, and without limitation, a number of particle, lipid, and/or polymer-based delivery systems are known in the art for delivery of siRNA. The invention contemplates use of such compositions. Liposomes or other lipid-based particles can also be used as pharmaceutically acceptable carriers.

Pharmaceutical compositions and compounds for use in such compositions may be manufactured under conditions that meet standards, criteria, or guidelines prescribed by a regulatory agency. For example, such compositions and compounds may be manufactured according to Good Manufacturing Practices (GMP) and/or subjected to quality control procedures appropriate for pharmaceutical agents to be administered to humans and can be provided with a label approved by a government regulatory agency responsible for regulating pharmaceutical, surgical, or other therapeutically useful products.

Pharmaceutical compositions of the invention, when administered to a subject for treatment purposes, are preferably administered for a time and in an amount sufficient to treat the disease or condition for which they are administered. Therapeutic efficacy and toxicity of active agents can be assessed by standard pharmaceutical procedures in cell cultures or experimental animals. The data obtained from cell culture assays and animal studies can be used in formulating a range of dosages suitable for use in humans or other subjects. Different doses for human administration can be further tested in clinical trials in humans as known in the art. The dose used may be the maximum tolerated dose or a lower dose. A therapeutically effective dose of an active agent in a pharmaceutical composition may be within a range of about 0.001 mg/kg to about 100 mg/kg body weight, about 0.01 to about 25 mg/kg body weight, about 0.1 to about 20 mg/kg body weight, about 1 to about 10 mg/kg. Other exemplary doses include, for example, about 1 μg/kg to about 500 mg/kg, about 100 μg/kg to about 5 mg/kg. In some embodiments a single dose is administered while in other embodiments multiple doses are administered. Those of ordinary skill in the art will appreciate that appropriate doses in any particular circumstance depend upon the potency of the agent(s) utilized, and may optionally be tailored to the particular recipient. The specific dose level for a subject may depend upon a variety of factors including the activity of the specific agent(s) employed, the particular disease or condition and its severity, the age, body weight, general health of the subject, etc. It may be desirable to formulate pharmaceutical compositions, particularly those for oral or parenteral compositions, in unit dosage form for ease of administration and uniformity of dosage. Unit dosage form, as that term is used herein, refers to physically discrete units suited as unitary dosages for the subject to be treated; each unit containing a predetermined quantity of active agent(s) calculated to produce the desired therapeutic effect in association with an appropriate pharmaceutically acceptable carrier. It will be understood that a therapeutic regimen may include administration of multiple doses, e.g., unit dosage forms, over a period of time, which can extend over days, weeks, months, or years. A subject may receive one or more doses a day, or may receive doses every other day or less frequently, within a treatment period. For example, administration may be biweekly, weekly, etc. Administration may continue, for example, until appropriate structure and/or function of a tissue or organ has been at least partially restored and/or until continued administration of the compound does not appear to promote further regeneration or improvement. In some embodiments, a subject administers one or more doses of a composition of the invention to him or herself.

In some embodiments, two or more compounds or compositions are administered in combination, e.g., for purposes of enhancing regeneration. Compounds or compositions administered in combination may be administered together in the same composition, or separately. In some embodiments, administration “in combination” means, with respect to administration of first and second compounds or compositions, administration performed such that (i) a dose of the second compound is administered before more than 90% of the most recently administered dose of the first agent has been metabolized to an inactive form or excreted from the body; or (ii) doses of the first and second compound are administered within 48, 72, 96, 120, or 168 hours of each other, or (iii) the agents are administered during overlapping time periods (e.g., by continuous or intermittent infusion); or (iv) any combination of the foregoing. In some embodiments, two or more iTR factors, or vectors expressing the catalytic component of telomerase and an iTR factor, are administered. In some embodiments an iTR factor is administered in combination with a combination with one or more growth factors, growth factor receptor ligands (e.g., agonists), hormones (e.g., steroid or peptide hormones), or signaling molecules, useful to promote regeneration and polarity. Of particular utility are organizing center molecules useful in organizing regeneration competent cells such as those produced using the methods of the present invention. In some embodiments, a growth factor is an epidermal growth factor family member (e.g., EGF, a neuregulin), a fibroblast growth factor (e.g., any of FGF1-FGF23), a hepatocyte growth factor (HGF), a nerve growth factor, a bone morphogenetic protein (e.g., any of BMP1-BMP7), a vascular endothelial growth factor (VEGF), a wnt ligand, a wnt antagonist, retinoic acid, NOTUM, follistatin, sonic hedgehog, or other organizing center factors.

### Sources of iTM and iCM Factors

iTM and iCM factors may be identified by exposing embryonic cells lacking markers of the EFT (such as, by way of nonlimiting example, stromal cells not expressing COX7A1) to a variety of agents and assaying for the induction of said markers such as COX7A1 or reporter constructs such as GFP expressed using the COX7A1 gene promoter.

Since exosomes carry potent protein and RNA factors capable of reprogramming cells to confer new growth, migration and differentiation properties, we examined whether they are capable of reprogramming the developmental state of a cell, i.e. iTM and iCM. We therefore tested exosomes from adult cells for induction of adult genes in embryonic cells. We assessed total RNA expression profile using Illumina microarray analysis of a series of 15 hESC derived clonal embryonic progenitor cell lines and compared these to 18 primary endothelial cell lines (newborn to adult) obtained from various anatomical sites (not shown). We determined that exosomes from cells that have passed the EFT are capable of inducing the expression of COX7A1 in embryonic cells previously lacking such expression, as well as maturing the cells using other markers described herein.

Those skilled in the art will recognize, or be able to ascertain using no more than routine experimentation, many equivalents to the specific embodiments of the invention described herein. The scope of the present invention is not intended to be limited to the Description or the details set forth therein. Articles such as “a”, “an” and “the” may mean one or more than one unless indicated to the contrary or otherwise evident from the context. Certain of the inventive methods are often practiced using populations of cells, e.g., in vitro or in vivo. Thus references to “a cell” should be understood as including embodiments in which the cell is a member of a population of cells, e.g., a population comprising or consisting of cells that are substantially genetically identical. However, the invention encompasses embodiments in which inventive methods is/are applied to an individual cell. Thus, references to “cells” should be understood as including embodiments applicable to individual cells within a population of cells and embodiments applicable to individual isolated cells.

Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. It is contemplated that all embodiments described herein are applicable to all different aspects of the invention. It is also contemplated that any of the embodiments can be freely combined with one or more other such embodiments whenever appropriate. Furthermore, it is to be understood that the invention encompasses all variations, combinations, and permutations in which one or more limitations, elements, clauses, descriptive terms, etc., from one or more of the claims (whether original or subsequently added claims) is introduced into another claim (whether original or subsequently added). For example, any claim that is dependent on another claim can be modified to include one or more elements or limitations found in any other claim that is dependent on the same base claim, and any claim that refers to an element present in a different claim can be modified to include one or more elements or limitations found in any other claim that is dependent on the same base claim as such claim. Furthermore, where the claims recite a composition, the invention provides methods of making the composition, e.g., according to methods disclosed herein, and methods of using the composition, e.g., for purposes disclosed herein. Where the claims recite a method, the invention provides compositions suitable for performing the method, and methods of making the composition. Also, where the claims recite a method of making a composition, the invention provides compositions made according to the inventive methods and methods of using the composition, unless otherwise indicated or unless one of ordinary skill in the art would recognize that a contradiction or inconsistency would arise. Where elements are presented as lists, e.g., in Markush group format, each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where phrases such as “less than X”, “greater than X”, or “at least X” is used (where X is a number or percentage), it should be understood that any reasonable value can be selected as the lower or upper limit of the range. It is also understood that where a list of numerical values is stated herein (whether or not prefaced by “at least”), the invention includes embodiments that relate to any intervening value or range defined by any two values in the list, and that the lowest value may be taken as a minimum and the greatest value may be taken as a maximum. Furthermore, where a list of numbers, e.g., percentages, is prefaced by “at least”, the term applies to each number in the list. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% or in some embodiments 10% of a number in either direction (greater than or less than the number) unless otherwise stated or otherwise evident from the context (e.g., where such number would impermissibly exceed 100% of a possible value). A “composition” as used herein, can include one or more than one component unless otherwise indicated. For example, a “composition comprising an activator or a TR activator” can consist or consist essentially of an activator of a TR activator or can contain one or more additional components. It should be understood that, unless otherwise indicated, an inhibitor or a TR inhibitor (or other compound referred to herein) in any embodiment of the invention may be used or administered in a composition that comprises one or more additional components including the presence of an activator of a TR activator.

### Novel Cancer Therapeutic Strategies

The methods and compositions of the present invention also provide for novel cancer therapeutics and companion diagnostics. Isoforms of the alpha and beta CPL are abundantly expressed in diverse types of embryonic cells up to the embryonic-fetal transition (i.e. pre-fetal) but, with the exception of some expression in CNS cells such as CNS neurons, are not expressed in diverse somatic cell types in subsequent fetal and adult development. Because the proteins encoded by the CPL isoform genes are expressed on the cell surface and exposed extracellularly, the present invention teaches that said proteins can be utilized as target antigens for cancer immunotherapy using members of the immunoglobulin superfamily that specifically recognize said alpha and beta CPL isoform proteins. By way of nonlimiting example, monoclonal or polyclonal antibodies to the proteins encoded by the alpha cluster genes: PCDHA1, PCDHA2, PCDHA3, PCDHA4, PCDHA5, PCDHA6, PCDHA7, PCDHA8, PCDHA9, PCDHA10, or PCDHA11, or the beta cluster genes: PCDHB1, PCDHB2, PCDHB3, PCDHB4, PCDHB5, PCDHB6, PCDHB7, PCDHB8, PCDHB9, PCDHB10, PCDHB11, PCDHB12, PCDHB13, PCDHB14, PCDHB15, PCDHB17P, PCDHB18P, or PCDHB19P may be administered to the patient to facilitate a humoral immune response to the cancer.

Alternatively, bi-specific antibodies that target an alpha or beta CPL isoform such as bi-specific T-cell engagers may be utilized to trigger an immune destruction specifically in cancer cells. Said bi-specific antibody may be composed, by way of nonlimiting example, of two single-chain variable fragments wherein one variable fragment binds to the target alpha or beta CPL isoform and the other to a T-Cell antigen such as CD3.

Alternatively, antibodies that target an alpha or beta CPL isoform may be conjugated to a toxin (antibody-drug conjugates) to specifically target and destroy tumor cells. The antibody-drug conjugates of the present invention include polyclonal, more preferably, monoclonal antibodies that target members of the alpha or beta CPL, and are chemically linked to a toxic payload. (Chao et al, 2019 The Lancet 394: 793-804; Teicher et al, 2022 Curr Cancer Drug Targets Feb 24 incorporated by reference). Said antibody-drug conjugate by way of nonlimiting example, may be IgA, IgD, IgE, IgG, or IgM chemically linked to a toxin such as DM4, monomethyl auristatin F (MMAF)), monomethyl auristatin E (MMAE), calicheamicin, DM1, using a linker such as valine-citrulline, Sulfo-SPDB, or hydrazone lysine-, cysteine-, or site-specific conjugation.

Alternatively, CAR T-cells may be utilized wherein the T-cell receptor is engineered to recognize an alpha or beta CPL isoform. Said CAR T-cells may be engineered into autologous T-cells and re-introduced into the patient, or more preferably, allogeneic CAR T-cells are produced from pluripotent stem cells such as iPSCs or hESCs in vitro, then introduced into the patient as adoptive immunotherapy for cancer.

Alternatively polyclonal, or more preferably, monoclonal antibodies specific to alpha or beta CPL isoform are conjugated to agents that facilitate in vivo imaging by MRI, SPECT, or PET imaging. Paramagnetic or superparamagnetic particles such as iron oxide may be conjugated to said antibodies for MRI. Radionuclides may be conjugated to said antibodies for nuclear imaging. ¹²⁴I and ⁸⁹Zr may be conjugated with said antibodies to image tumors by PET. These and related imaging techniques using the specific expression of alpha or beta CPL isoforms in cancer are useful in detecting and diagnosing cancer, as well as providing useful companion diagnostic data on the extent of tumor reduction following a therapeutic regimen, including those described herein.

Furthermore, the present invention teaches that certain molecular pathways associated with the EFT evolved in part as a method to restrain the replication of endogenous transposable elements and viruses including Class I transposable elements (retrotransposons), Class II transposable elements (DNA transposons), LINES, SINES, as well as other viruses such as retroviruses. Prior to the EFT and in mammalian pre-implantation embryos, some cells, such as cells of the inner cell mass or cells isolated from the inner cell mass such as cultured hES cells, are permissive for viral replication. The relative permissivity of some embryonic (pre-fetal) cells to endogenous transposable element replication is known in the art. For example, it is documented that human endogenous retroviruses such as HERVK replicate in some pluripotent stem cell lines (Grow, E. J. et al, (2015) Nature 522:221-225). However, the association of Lamin-A with the EFT and the suppression of viral replication has not been described.

The present invention teaches that lamin-A, in particular, its processing into mature filaments and association with LRRK2 and PLPP7 evolved as a means of guarding the integrity of the genome, in particular, regions of repetitive sequences such as those associated with telomeric repeats and tandemly-repeated paralogs such as those of the clustered protocadherin locus or regions of tandemly-repeated paralogs of zinc finger proteins that evolved to inactivate diverse viral sequences. In addition, Lamin A evolved as a means of limiting the plasticity of diverse differentiated somatic types, that is, stabilizing them in their differentiated state. In limiting their plasticity, it limited the potential of diverse somatic cell types and tissues to regenerate after injury or disease by utilizing diverse pathways. These pathways included the downregulation of the embryonic cell-cell recognition system of the clustered protocadherin locus and increased signaling associated with the epithelial-mesenchymal transformation (EMT) such as increased expression of extracellular matrix proteins such as those encoded by the genes: FN1, COL1A1, SPARC, and VIM that result in a fibrotic scarring of adult tissue in lieu of regeneration as seen in embryonic tissue following injury. As a result, Lamin A plays an important regulatory role as an inhibitor of tissue regeneration (See, e.g. U.S. provisional patent application No. 63/155,628, filed Mar. 2, 2021, the disclosure of which is incorporated by reference in its entirety), but also the formation of cancer stem cells (CSC) which have been disclosed to be not a more undifferentiated cell type as is the current consensus belief, but rather a more mature cell type corresponding to fetal/adult cells, as opposed to the embryonic (pre-fetal) state of many malignant cell types from diverse somatic cell origins.

### Oncolytic Viral Therapy

The permissive state of pre-EFT somatic cells therefore is consistent with the permissive replication of diverse viruses in cancer cells. While there are currently no efficient means of determining in advance which tumors or cancer cells types will be efficiently destroyed by said vectors, the embryonic and adult gene expression markers in previously disclosures (See, e.g. U.S. provisional patent application No. 61/831,421, filed Jun. 5, 2013, PCT patent application PCT/US2014/040601, filed Jun. 3, 2014 and U.S. Pat. No. 10,961,531, filed on Dec. 7, 2015, PCT patent application PCT/US2017/036452, filed Jun. 7, 2017 and U.S. patent application Ser. No. 16/211,690, filed on Dec. 6, 2018, and U.S. provisional patent application No. 63/256,286, filed Oct. 15, 2021, the disclosures of which are incorporated by reference in their entirety), as well as the differentially-methylated DNA sequences associated with embryonic vs fetal/adult cells (see, e.g. PCT patent application PCT/US2020/047707, filed Aug. 25, 2020, the disclosure of which is incorporated by reference in its entirety), provide useful means of determining which cancer cells or tumors will respond to oncolytic viral therapy. Cancer cells or tumors that express embryonic (pre-fetal) markers such as a lack of COX7A1 expression, relatively low expression of LMNA, or alternatively express embryonic (pre-fetal) markers such as the expression of PCAT7, are permissive for the replication of viruses and are therefore sensitive to oncolytic viral therapy. In addition, methods of inducing tissue regeneration such as those disclosed in (see, e.g. U.S. provisional patent application No. 61/831,421, filed Jun. 5, 2013, PCT patent application PCT/US2014/040601, filed Jun. 3, 2014 and U.S. Pat. No. 10,961,531, filed on Dec. 7, 2015, e.g. PCT patent application PCT/US2017/036452, filed Jun. 7, 2017 and U.S. patent application Ser. No. 16/211,690, filed on Dec. 6, 2018, U.S. provisional patent application No. 63/155,628, filed Mar. 2, 2021, and U.S. provisional patent application No. 63/256,286, filed Oct. 15, 2021, the disclosures of which are incorporated by reference in their entirety) are useful in transforming CSCs into their embryonic counterparts wherein the cancer cells will be responsive to oncolytic viral therapy.

The novel oncolytic viral therapies of the present invention include the use of viruses currently-disclosed as selectively destroying malignant cancer cells including: Herpes Simplex Virus Type I (HSV-1) such as Talimogene laherparepvec (T-VEC) modified to express GM-CSF with a promoter of an embryonic (pre-fetal) gene promoter such as the PCAT7, CPT1B, or PURPL promoters or other embryonic promoters previously disclosed (See, e.g. U.S. provisional patent application No. 63/256,284, filed Oct. 15, 2021, the disclosure of which is incorporated by reference in its entirety).

In addition, viruses useful in targeting cancer cells such as HSV-1, reovirus, picornaviruses (coxsackeievirus, rigavirus) rhabdoviruses such as vesicular stomatitis virus and Maraba virus, and paramyxoviruses such as Newcastle disease virus and Measles virus, and vaccinia virus may be modified to express toxic gene products or genes useful to express specifically in cancer cells such as GM-CSF that are useful in promoting dendritic cell activation wherein said introduced genes are expressed from a gene promoter such as the PCAT7, CPT1B, or PURPL promoters or other embryonic promoters previously disclosed (See, e.g. U.S. provisional patent application No. 63/256,284, filed Oct. 15, 2021, the disclosure of which is incorporated by reference in its entirety).

In addition, viruses useful in targeting cancer cells such as HSV-1, reovirus, picornaviruses (coxsackeievirus, rigavirus) rhabdoviruses such as vesicular stomatitis virus and Maraba virus, and paramyxoviruses such as Newcastle disease virus and Measles virus, and vaccinia virus may be modified to express RNAi to zinc finger protein genes that are activated in fetal/adult cells wherein said zinc finger proteins inhibit viral replication. As a result, infected cells, such as cancer cells with an fetal/adult-like phenotype are rendered more susceptible to lysis. Said fetal/adult-onset zinc finger genes activated by Lamin A include: ZNF280D (See, e.g. U.S. provisional patent application No. 61/831,421, filed Jun. 5, 2013, PCT patent application PCT/US2014/040601, filed Jun. 3, 2014 and U.S. Pat. No. 10,961,531, filed on Dec. 7, 2015, the disclosures of which are incorporated by reference in their entirety), ZNF300P1, ZNF-572 (See, e.g. PCT patent application PCT/US2017/036452, filed Jun. 7, 2017 and U.S. patent application Ser. No. 16/211,690, filed on Dec. 6, 2018, the disclosures of which are incorporated by reference in their entirety), and ZNF578, ZNF585B, ZNF736, and ZNF790-AS1 (See, e.g. U.S. provisional patent application No. 63/256,286, filed Oct. 15, 2021, the disclosure of which is incorporated by reference in its entirety).

In addition, the present invention provides for novel oncolytic viral therapy which when used alone or in combination with immune checkpoint inhibition, or adoptive immunotherapy, are useful in selectively destroying cancer cells with an embryonic phenotype. Numerous immune checkpoint inhibitors useful in treating cancer are known in the art and may be utilized as a combination therapy with the cancer therapeutics described herein. Nonlimiting examples of immune checkpoint inhibitors antibodies targeting PD-1 such as Nivolumab, Cemiplimab, Spartalizumab, and Pembrolizumab and antibodies targeting PD-L1 such as Atezolizumab, Avelumab, and Durvalumab, and antibodies targeting CTLA4 such as Ipilimumab. Additional immune checkpoint inhibition can be achieved by T-Cell Adoptive Cancer Immunotherapy. Said T-Cells are used wherein they express decreased levels of or have a knock-out of CISH (cytokine-inducible SH2-containing protein) or CBLB (Cbl Proto-oncogene, E3 Ubiquitin Protein Ligase B).

Additional combinations that are useful in achieving greater levels of reduction in tumor burden can be achieved by combining the oncolytic viruses of the present invention with the above mentioned immune checkpoint inhibitors, together with dendritic cell therapy and/or CAR-T cells targeting embryonic (pre-fetal) antigens such as those described herein.

The phenotypic alterations of the EFT are shared in common with the majority of all somatic cell types. Similarly, the abnormal embryonic phenotype (embryo-onco phenotype) of many cancer cells and the fetal/adult phenotype of CSCs are shared by many cancer types (i.e. are pan-cancer phenotypic alterations). They are useful in the diagnosis and treatment of primary and metastatic cancers including: Acanthoma, Acinar adenocarcinoma, Acinic cell carcinoma, Acrospiroma, Acute eosinophilic leukemia, Acute erythroid leukemia, Acute Lymphoblastic Leukemia (ALL), Acute megakaryoblastic leukemia, Acute monocytic leukemia, Acute Myeloid Leukemia (AML), Acute promyelocytic leukemia, Adamantinoma, Adenoid cystic carcinoma, Adenomatoid odontogenic tumor, Adenosquamous carcinoma, Adenosquamous lung carcinoma, Adipose tissue neoplasm, Adrenocortical carcinoma, Adrenocortical carcinoma childhood, Aggressive NK-cell leukemia, AIDS-related cancers, Alveolar rhabdomyosarcoma, Alveolar soft part sarcoma, Ameloblastic fibroma, Anal cancer, Anaplastic carcinoma, Anaplastic large-cell lymphoma, Anaplastic thyroid cancer, Angioimmunoblastic T-cell lymphoma, Angiosarcoma, Appendix cancer, Attenuated familial adenomatous polyposis, Atypical teratoid/rhabdoid tumor central nervous system childhood, B-cell chronic lymphocytic leukemia, B-cell lymphoma, Bellini duct carcinoma, Bile duct cancer, Bile duct cancer—Cholangiocarcinoma, Bladder cancer, Bladder cancer—Small cell carcinoma, Bladder cancer—Transitional cell carcinoma, Bladder cancer childhood, Blastoma, Bone cancer, Bone cancer—Osteosarcoma, Brain stem glioma, Brain tumors —other, Brain tumor—Glioblastoma multiforme, Brain tumor—Oligodendroglioma anaplastic, Brain tumor—cerebellar astrocytoma (childhood & adult), Brain tumor—cerebral astrocytoma/malignant glioma (childhood & adult), Brain tumor—ependymoma, Brain tumor—medulloblastoma, Brain tumor—supratentorial primitive neuroectodermal tumors, Brain tumor—visual pathway and hypothalamic glioma, Brain and spinal cord tumors childhood, Breast cancer, Breast cancer ductal adenocarcinoma, Breast cancer childhood, Brenner tumour, Bronchial adenomas/carcinoids, Bronchial tumors, Bronchial tumors childhood, Bronchioloalveolar carcinoma, Brown tumor, Burkitt lymphoma, Carcinoid tumor, Carcinoid tumor childhood, Carcinoid tumor gastrointestinal, Carcinoma of the penis, Carcinosarcoma, Cementoma, Central nervous system cancer, Cervical cancer—adenocarcinoma, Cervical cancer—squamous cell, Cervical Cancer—Neuroendocrine, Carcinoma of the cervix, Cervical cancer childhood, Childhood cancers, Childhood leukemia, Cholangiocarcinoma, Cholangiosarcoma, Chondromyxoid fibroma, Chondrosarcoma, Chordoma, Chorioadenoma destruens, Chorioblastoma, Choriocarcinoma, Choroid plexus tumor, Chorioepithelioma, Clear cell adenocarcinoma, Clear cell adenocarcinoma of the vagina, Clear-cell ovarian carcinoma, Clear-cell sarcoma of the kidney, Colon cancer, Colon cancer—adenocarcinoma, Colorectal cancer, Colorectal cancer childhood, Comedocarcinoma, Craniopharyngioma, Craniopharyngioma childhood, Cutaneous lymphoma, Cystadenocarcinoma, Degos disease, Dermatofibrosarcoma protuberans, Desmoplastic small round cell tumor, Diffuse large B-cell lymphoma, Digestive system neoplasm, Diktyoma, Ductal carcinoma In situ (DCIS), “Ductal, lobular, and medullary neoplasms”, Duodenal cancer, Dysembryoplastic neuroepithelial tumour, Dysgerminoma, ELM4-ALK positive lung cancer, Embryoma, Embryonal carcinoma, Embryonal rhabdomyosarcoma, Embryonal tumors central nervous system childhood, Endocrine gland neoplasm, Endodermal sinus tumor, Endometrial cancer, Endometrial—Stromal sarcoma, Endometrial—Adenocarcinoma, Endometrioid tumor, Enteropathy-associated T-cell lymphoma, Ependymoblastoma childhood, Ependymoma childhood, Epithelial-myoepithelial carcinoma of the lung, Epithelioid sarcoma, Epithelioma, Esophageal cancer, Esophageal cancer childhood, Esthesioneuroblastoma childhood, Ewing family of tumors, Ewing's sarcoma in the Ewing family of tumors, Exocrine cancer, Extracranial germ cell tumor childhood,Extragonadal germ cell tumor, Extrahepatic bile duct cancer, Extramammary Paget's disease, Eye cancer, “Eye cancer, intraocular melanoma”, “Eye cancer, retinoblastoma”, Fallopian tube cancer, Familial adenomatous polyposis, Fetal adenocarcinoma, Fibroepithelial neoplasms, Fibrolamellar hepatocellular carcinoma, Fibrosarcoma, Fibrous tissue neoplasm, Follicular lymphoma, Follicular thyroid cancer, GCB Diffuse Large B-Cell Lymphoma (DLBCL), Gallbladder cancer, Ganglioglioma, Ganglioneuroma, Gardner's syndrome, Gastric carcinoid, Gastric (stomach) cancer, Gastric (stomach) cancer—Adenocarcinoma, Gastric (stomach) cancer—Adenocarcinoma of gastroesophageal junction, Gastric (stomach) cancer childhood, Gastric lymphoma, Gastrinoma, Gastrointestinal carcinoid tumor, Gastrointestinal stromal tumors (GIST), Germ cell tumor, Extragonadal germ cell tumor, Ovarian germ cell tumor, Germinoma, Gestational choriocarcinoma, Gestational trophoblastic tumor, Giant-cell fibroblastoma, Giant-cell glioblastoma, Giant-cell tumor of bone, Gigantiform cementoma, Glial tumor, Gliomatosis cerebri, Glioblastoma Multiforme, Glioma, Glioma childhood visual pathway and hypothalamic, Gliosarcoma, Glucagonoma, Goblet cell carcinoid, Gonadoblastoma, Granulosa cell tumour, Gynandroblastoma, Head and neck cancer, Head and neck cancer childhood, Heart cancer, Hemangioblastoma, Hemangiopericytoma, Hemangiosarcoma, Hematological malignancy, Hepatic cancer—Cholangiocarcinoma, Hepatoblastoma, Hepatocellular (liver) cancer, Hepatosplenic T-cell lymphoma, Hereditary breast-ovarian cancer syndrome, Hereditary nonpolyposis colorectal cancer, Histiocytic sarcoma, Histiocytoma, Hypopharyngeal cancer, Inflammatory breast cancer, Inflammatory myeloblastic tumor, Intraductal carcinoma, Intraductal papillary mucinous neoplasm, Intraocular melanoma, Intratubular germ cell neoplasia, Invasive lobular carcinoma, Islet cell carcinoma, Islet cell tumors (endocrine pancreas), Juvenile granulosa cell tumor, Juvenile myelomonocytic leukemia, Juxtaglomerular cell tumor, Kaposi sarcoma, Kidney cancer childhood, Klatskin tumor, Krukenberg tumor, Langerhans cell histiocytosis, Large-cell lung carcinoma with rhabdoid phenotype, Laryngeal cancer, Laryngeal cancer—squamous cell carcinoma, Laryngeal cancer childhood, Leiomyosarcoma, Lentigo malignant melanoma, Leptomeningeal cancer, Leukemias, Leydig cell tumour, Chronic lymphocytic leukemia (CLL), Chronic myelogenous leukemia (CML), Hairy cell leukemia, Linitis plastica, Lip and oral cavity cancer, Liposarcoma, Liver cancer (primary), Lobular carcinoma, Lobular carcinoma In situ (LCIS), Giant-cell carcinoma of the lung, Large-cell lung carcinoma, Large-cell lung carcinoma with rhabdoid phenotype, Non-small cell lung cancer, Lung—Adenocarcinoma, Lung—Large cell_carcinoma, Lung—Small cell_carcinoma, Lung—Squamous cell_carcinoma, Luteoma, Lymphangioma, Lymphangiosarcoma, Lymphoepithelioma, Lymphomas, Lymphoma—Extranodal marginal zone B-cell of lymphoid tissue, Lymphoma—Follicular cancer of lymphoid tissue, AIDS-related_lymphoma, Cutaneous T-cell lymphoma, Hodgkin_lymphoma, Non-hodgkin lymphoma, Primary central nervous system lymphoma (CNS), Macroglobulinemia Waldenstrîm, Male breast cancer, Malignant fibrous histiocytoma of bone and osteosarcoma, Malignant peripheral nerve sheath tumor, Malignant triton tumor, MALT lymphoma, Mammary ductal carcinoma, Mantle cell lymphoma, Marginal zone B-cell lymphoma, “Marcus Whittle, deadly disease”, Mast cell leukemia, Mediastinal germ cell tumor, Mediastinal tumor, Medullary carcinoma, Medullary carcinoma of the breast, Medullary thyroid cancer, Medulloblastoma, Medulloblastoma childhood, Medulloepithelioma, Medulloepithelioma childhood, Melanoma, Melanoma childhood, Meningioma, Merkel cell carcinoma, Mesenchymal chondrosarcoma, Mesothelioma adult malignant, Mesothelioma adult malignant—pleural mixed, Mesothelioma childhood, Metastatic breast cancer, Metastatic squamous neck cancer with occult primary, Metastatic tumor of jaws, Metastatic urothelial carcinoma, Mixed Mullerian tumor, Mouth cancer, Mucinous cystadenocarcinoma of the lung, Mucinous tumor, Multiple endocrine neoplasia syndromes childhood, Multiple endocrine neoplasia type 2b, Multiple myeloma/plasma cell neoplasm, Muscle tissue neoplasm, Mycosis fungoides, Myelodysplastic/myeloproliferative neoplasms, Myelodysplastic syndromes, Myeloid leukemia adult acute, Myeloid leukemia childhood acute, Myeloid sarcoma, Chronic myeloproliferative disorders, Myosarcoma, Myxoid chondrosarcoma, Myxoid liposarcoma, Myxoma, Myxosarcoma, Nasal cavity and paranasal sinus cancer, Nasopharyngeal angiofibroma, Nasopharyngeal cancer, Nasopharyngeal cancer childhood, Nerve sheath tumor, Nervous system neoplasm, Neuroblastoma, Neurocytoma, Neurofibroma, Neuroma, Nipple adenoma, Nodular lymphocyte predominant Hodgkin's lymphoma, Nodular melanoma, Odontogenic tumor, Oncocytoma, Optic nerve sheath meningioma, Optic nerve tumor, Oral cancer, Oral cancer childhood, Oropharyngeal cancer, Oropharyngeal squamous cell carcinomas, Osteolipochondroma, Osteoma, Osteosarcoma, Ovarian cancer, Ovarian cancer—Adenocarcinoma of ovary serous, Ovarian cancer childhood, Ovarian cancer epithelial, Ovarian cancer germ cell tumor, Paget's disease of the breast, Pancoast tumor, Pancreatic cancer, Pancreatic cancer childhood, Pancreatic cancer—Neuroendocrine, Pancreatic cancer islet cell tumors, Pancreatic—Adenocarcinoma of pancreas ductal, Pancreatic serous cystadenoma, Papillary adenocarcinoma, Papillary serous cystadenocarcinoma, Papillary thyroid cancer, Papillomatosis childhood, Paraganglioma, Parathyroid adenoma, Parathyroid cancer, Parathyroid neoplasm, PEComa, Periampullary cancer, Peritoneal mesothelioma, Pharyngeal Cancer, Pheochromocytoma, Pineal astrocytoma, Pineal germinoma, Pineal parenchymal tumors of intermediate differentiation childhood, Pinealoblastoma, Pineoblastoma and supratentorial primitive neuroectodermal tumors childhood, Pineocytoma, Pituicytoma, Pituitary adenoma, Pituitary tumor, Plasma cell dyscrasia, Plasma cell leukemia, Plasma cell neoplasm/multiple myeloma, Plasmacytoma, Pleomorphic undifferentiated sarcoma, Pleomorphic xanthoastrocytoma, Pleuropulmonary blastoma, Pleuropulmonary blastoma childhood, Polyembryoma, Posterior urethral cancer, Precursor T-lymphoblastic lymphoma, Primary peritoneal carcinoma, Primitive neuroectodermal tumor, Prostate cancer, Prostate cancer—adenocarcinoma, Pseudomyxoma peritonei, Rectal cancer, Rectal cancer—adenocarcinoma, Renal cell carcinoma (kidney cancer), Renal medullary carcinoma, Renal pelvis and ureter transitional cell cancer, Reninoma, Respiratory tract neoplasm, Retinoblastoma, Rhabdomycin, Rhabdomyosarcoma childhood, Richter's transformation, Salivary gland cancer, Salivary gland cancer childhood, Salivary gland-like carcinoma of the lung, Salivary gland neoplasm, Sacrococcygeal teratoma, Sarcoma, Sarcoma botryoides, Sarcoma soft tissue, Sarcomatoid carcinoma, Schwannomatosis, Sclerosing rhabdomyosarcoma, Secondary neoplasm, Seminoma, Serous carcinoma, Serous cystadenocarcinoma, Serous tumour, Sertoli cell tumour, Sertoli-Leydig cell tumour, Sex cord-gonadal stromal tumour, Sçzary syndrome, Signet ring cell carcinoma, Skin cancer, Skin cancer childhood, Skin cancer—basal cell carcinoma, Skin cancer—basal-like carcinoma, Skin cancer—melanoma, Small-cell carcinoma, Small intestine cancer, “Small-, round-, blue-cell tumour”, Somatostatinoma, Soot wart, Spermatocytic seminoma, Spinal tumor, Spindle cell cancer, Spindle cell rhabdomyosarcoma, Splenic lymphoma with villous lymphocytes, Splenic marginal zone lymphoma, Squamous cell carcinoma, Squamous neck cancer with occult primary metastatic, Stewart_Treves syndrome, Stromal tumor, Supratentorial primitive neuroectodermal tumors childhood, Surface epithelial-stromal tumor, Synovial sarcoma, T-cell lymphoma, T-lymphoblastic lymphoma, Teratocarcinoma, Testicular cancer, Testicular cancer—Seminoma, Testicular cancer childhood, Thecoma, Throat cancer, “Thymoma, childhood”, Thymoma and thymic carcinoma, Thymoma and thymic carcinoma childhood, Thyroid cancer, Thyroid cancer—follicular, Thyroid cancer—papillary, Thyroid cancer childhood, Tonsil—Carcinoma of tonsil squamous cell, Trabecular cancer, Tracheal tumor, Transitional cell carcinoma, Trophoblastic tumor gestational, Tubulovillous adenoma, Urachal cancer, Ureteral cancer, Ureteral neoplasm, Urethral cancer, Urogenital neoplasm, Urothelial carcinoma, Urothelial cell carcinoma, Uterine cancer, Uterine cancer endometrial, Uterine clear cell carcinoma, Uterine sarcoma, Uterine serous carcinoma, Uveal melanoma, Vaginal cancer, Vaginal cancer childhood, Verrucous carcinoma, Vestibular schwannoma, VIPoma, Visual pathway glioma, Von Hippel_Lindau disease, Vulvar Cancer, “Wilms tumor (kidney cancer), childhood”.

### EXAMPLES

Certain conventional techniques of cell biology, cell culture, molecular biology, microbiology, recombinant nucleic acid (e.g., DNA) technology, immunology, etc., which are within the skill of the art, may be of use in aspects of the invention. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., editions as of 2008; Sambrook, Russell, and Sambrook, Molecular Cloning: A Laboratory Manual, 3.sup.rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Burns, R., Immunochemical Protocols (Methods in Molecular Biology) Humana Press; 3rd ed., 2005, Monoclonal antibodies: a practical approach (P. Shepherd and C Dean, eds., Oxford University Press, 2000); Freshney, R. I., “Culture of Animal Cells, A Manual of Basic Technique”, 5th ed., John Wiley & Sons, Hoboken, N J, 2005). All patents, patent applications, websites, databases, scientific articles, and other publications mentioned herein are incorporated herein by reference in their entirety.

### Methods

In addition to the methods described below, methods that find use in the production and use of cells with an embryonic pattern of gene expression corresponding with scarless regenerative potential can be found in the following: PCT application Ser. No. PCT/US2006/013519; U.S. patent application Ser. No. 11/604,047; and U.S. patent application Ser. No. 12/504,630, (See, e.g. PCT Patent Application Ser. No. PCT/US2014/040601, and U.S. patent application Ser. No. 14/896,664, each of which is incorporated by reference in its entirety), each of which is incorporated by reference herein in its entirety.

### Cell Culture

Pluripotent stem cells (PSCs) (hESCs and iPSCs) were cultured on Matrigel in mTeSR1 medium in a humidified incubator at 37° C. with 5% O₂ and 10% CO₂, Embryonic Progenitor Cells (PCs) were cultured on 0.1% gelatin in their specific growth medium used originally when cloning and scaling the line in a humidified incubator at 37° C. with 5% O₂ and 10% CO₂. The EP cell lines 4D20.8 and were cultured in DMEM 20% FBS and PromoCell endothelial (MV2) growth medium respectively. Cells were routinely passaged 1:3 at or near confluence using 0.05% trypsin. Adult-derived cells were cultured in their respective optimal growth mediums on 0.1% gelatin in Corning cultureware at 37° C. with 5% O₂ and 10% CO₂ or in select cases, total RNA was purchased from ScienCell (Carlsbad CA). The cancer cell lines were obtained from ATCC and cultured as suggested by ATCC prior to harvest. Prior to analysis, all lines were synchronized in quiescence when possible by culturing five days in 10% of the normal serum or related growth factor supplements.

### RNA Isolation

RNA was prepared upon lysis with RLT with 1% 2-PME, using Qiagen RNeasy mini kits (Cat #74104) following manufacturer's directions. The extracted RNA was then quantitated using a NanoDrop (ND-1000) spectrophotometer and the labeled tubes were stored at −80° C. for later use.

### RNA-Sequencing

Library Construction was performed by using Illumina Truseq mRNA library prep kit following manufacturer's directions. Library QC and library pooling was accomplished using Agilent Technologies 2100 Bioanalyzer™ to assay the library fragments. qPCR was used to quantify the libraries. Libraries were pooled, which have different barcodes/indexing and sequencing, in one lane. The paired-end sequencing was performed using the Illumina HiSeq4000™ sequencing instrument, yielding 100-bp paired-end reads. The sequencing was performed by BGI AMERICAS CORPORATION.

### RNA-Seq Data Analysis

The fastq files containing a minimum of 25 million reads per sample obtained by sequencing were analyzed using the Tuxedo protocol⁶². Reads are aligned against GRCh38 using short read aligner Bowtie2 (release 2.2.7) within the TopHat (release 2.1.1) splice junction mapper. Bowtie2 indices, as well as GRCh38 genome annotation) were obtained from Illumina, Inc. iGenomes. Alignment files were assembled into transcripts, and the abundances estimated using cufflinks 2.2.1 release. To allow for high abundance of transcripts, the parameter—max bundle-frags 2000000 was used. Cufflinks gtf files were merged with genes.gtf of GRCh38 annotation using Cuffmerge (release 2.2.1) into unified transcript catalog. Transcript abundance levels were computed using Cuffquant, and the resulting data normalized using Cuffnorm (both 2.2.1 Cufflinks release).

### Volcano Plot

Data analysis of the transcription levels (FPKM values) was carried out in R. Data was filtered to remove genes from the Y chromosome. FPKM values were rounded to two decimal places and low-expressing entities were removed by filtered for entities that had a mean FPKM value>0.5 in either the embryonic or adult group and a mean>0 in both groups. P-values were generated using a t-test with Bonferroni correction. Plots were generated in R.

### Statistical Analysis

Statistical significance of differences in FPKM values was determined using a two-tailed T-test assuming two samples of equal variance (homoscedastic). Error bars designate standard error of the mean unless otherwise noted.

### ATAC-seq

Embryonic progenitor cell lines (4D20.8 and 30-MV2-6), adult cell lines (MSC and HAEC), PSC lines, and cancer lines were harvested and frozen in culture media containing FBS and 5% DMSO. Cryopreserved cells were sent to Active Motif to perform the ATAC-seq assay. The cells were then thawed in a 37° C. water bath, pelleted, washed with cold PBS, and tagmented as previously described²¹,6³. Briefly, cell pellets were resuspended in lysis buffer, pelleted, and tagmented using the enzyme and buffer provided in the Nextera™ Library Prep Kit (Illumina). Tagmented DNA was then purified using the MinElute PCR purification kit (Qiagen), amplified with 10 cycles of PCR, and purified using Agencourt AMPure SPRI™ beads (Beckman Coulter). Resulting material was quantified using the KAPA Library Quantification Kit for Illumina platforms (KAPA Biosystems), and sequenced with PE42 sequencing on the NextSeq 500 sequencer (Illumina).

Analysis of ATAC-seq data was very similar to the analysis of ChIP-Seq data. Reads were aligned using the BWA algorithm (mem mode; default settings). Duplicate reads were removed, only reads mapping as matched pairs and only uniquely mapped reads (mapping quality >=1) were used for further analysis. Alignments were extended in silico at their 3′-ends to a length of 200 bp and assigned to 32-nt bins along the genome. The resulting histograms (genomic “signal maps”) were stored in bigWig files. Peaks were identified using the MACS 2.1.0 algorithm at a cutoff of p-value 1e-7, without control file, and with the—nomodel option. Peaks that were on the ENCODE blacklist of known false ChIP-Seq peaks were removed. Signal maps and peak locations were used as input data to Active Motifs proprietary analysis program, which creates Excel tables containing detailed information on sample comparison, peak metrics, peak locations and gene annotations.

### TOBIAS Analysis

To qualify the potential bindings of the transcription factors to the open regions characterized by ATAC-Seq, we used the TOBIAS algorithm⁶⁴. Histogram peaks were characterized from the BAM files using MACS2 callpeak function of MACS⁶⁵. To compensate for Tn5 transposase site insertion bias., TOBIAS ATACorrect function was used, and the data stored in bigwig output files. Corrected bigwig files were characterized for peaks using MACS2 call peaks function with the following parameters: —shift-100-extsize 200-broad. Obtained peaks were searched for the transcription factor binding footprints using TOBIAS Footprint. Scores function. Binding site footprint scores were characterized for transcription factor binding events with TOBIAS BIN Detect for the pairs of samples (4D20 vs HMSC, and 30MV2-6 vs HAEC) against JASPAR 2020 database⁶⁶. Resulting bed files were visualized by IGV⁶⁷.

### Chromatin Immunoprecipitation

Cells were fixed with 1% formaldehyde for 15 min and quenched with 0.125 M glycine. Frozen cell pellets were sent to Active Motif to perform the ChIP-Seq assay. Chromatin was isolated by the addition of lysis buffer, followed by disruption with a Dounce homogenizer. Lysates were sonicated and the DNA sheared to an average length of 300-500 bp. Genomic DNA (Input) was prepared by treating aliquots of chromatin with RNase, proteinase K and heat for de-crosslinking, followed by ethanol precipitation. Pellets were resuspended and the resulting DNA was quantified on a NanoDrop spectrophotometer. Extrapolation to the original chromatin volume allowed quantitation of the total chromatin yield.

An aliquot of chromatin (30 μg) was precleared with protein A or G agarose beads (Invitrogen). Genomic DNA regions of interest were isolated using 4 ug of antibody against the specific histone modification of interest. Complexes were washed, eluted from the beads with SDS buffer, and subjected to RNase and proteinase K treatment. Crosslinks were reversed by incubation overnight at 65° C., and ChIP DNA was purified by phenol-chloroform extraction and ethanol precipitation.

Quantitative PCR (QPCR) reactions were carried out in triplicate on specific genomic regions using SYBR Green Supermix (Bio-Rad). The resulting signals were normalized for primer efficiency by carrying out QPCR for each primer pair using Input DNA.

### ChIP Sequencing

Illumina sequencing libraries were prepared from the ChIP and Input DNAs by the standard consecutive enzymatic steps of end-polishing, dA-addition, and adaptor ligation. Steps were performed on an automated system (Apollo 342, Wafergen Biosystems/Takara). After a final PCR amplification step, the resulting DNA libraries were quantified and sequenced on Illumina's NextSeq 500 (75 nt reads, single end). Reads were aligned to the human genome (hg38) using the BWA algorithm (default settings). Duplicate reads were removed and only uniquely mapped reads (mapping quality >=25) were used for further analysis. Alignments were extended in silico at their 3′-ends to a length of 200 bp, which is the average genomic fragment length in the size-selected library, and assigned to 32-nt bins along the genome. The resulting histograms (genomic “signal maps”) were stored in bigWig files. For active histone marks, peak locations were determined using the MACS algorithm (v2.1.0) with a cutoff of p-value=1e-7. For repressive histone marks or histone modifications with broad distribution, enriched regions were identified using the SICER algorithm at a cutoff of FDR 1E-10 and a max gap parameter of 600 bp. Peaks that were on the ENCODE blacklist of known false ChIP-Seq peaks were removed. Signal maps and peak locations were used as input data to Active Motifs proprietary analysis program, which creates Excel tables containing detailed information on sample comparison, peak metrics, peak locations and gene annotations.

### Whole-Genome Bisulfite Sequencing

DNA was prepared from cell lines using Qiagen DNeasy Blood and Tissue kits (Cat #69504) following manufacturer's instructions. The labeled microfuge tubes were then stored at −80° C. Samples were sequenced as 150-bp paired-end reads, to the level of about 90G of raw data per sample. Sequencing was performed by BGI AMERICAS CORPORATION.

WGBS data analysis

Whole genome bisulfite sequencing data analysis was performed on obtained fastq reads files using Bismark suite with default parameters⁶⁸.

### Identification of DMRs

DMRs were identified from the WGBS data using Metilene software⁶⁹. DMRs were defined with a q-value <0.01 and a mean methylation difference >0.15 in a window of at least 250 nt, eight CpGs, and signal in at least three of four of the embryonic or adult cell lines in the cohort.

### TAD Method

Chromatin conformation capture data was generated using a Phase Genomics (Seattle, WA) Proximo Hi-C 2.0 Kit, which is a commercially available version of the Hi-C protocol⁷⁰. Following the manufacturer's instructions for the kit, intact cells from two samples were crosslinked using a formaldehyde solution, digested using the Sau3AI restriction enzyme, and proximity ligated with biotinylated nucleotides to create chimeric molecules composed of fragments from different regions of the genome that were physically proximal in vivo, but not necessarily genomically proximal. Continuing with the manufacturer's protocol, molecules were pulled down with streptavidin beads and processed into an Illumina-compatible sequencing library. Sequencing was performed on an Illumina HiSeq 4000.

Reads were aligned to the Homo sapiens genome assembly GRCh38 (hg38), also following the manufacturer's recommendations. Briefly, reads were aligned using BWA-MEM⁷¹ with the −5SP and −t 8 options specified, and all other options default. SAMBLASTER⁷² was used to flag PCR duplicates, which were later excluded from analysis. Alignments were then filtered with samtools⁷³ using the −F 2304 filtering flag to remove non-primary and secondary alignments. TopDom™⁷⁴ was used to identify topologically associated domains (TADs) at a 50 kb resolution by computing the average contact frequency among pairs of chromatin regions (one upstream and one downstream) in a small window (w=5) around the 50 kb bin. The resulting curve was used to identify local minima, or regions of low chromatin contact, along each chromosome. Regions of interest were plotted using pyplot and patches from the matplotlib Python package⁷⁵. The BED files documenting TAD calls by TopDom were simplified and viewed as tracks in the USCS genome browser. Data visualization of obtained .hic files were visualized using the Juicebox suite.

Induced Senolysis of Cancer Stem Cells (iS-CSC)

In addition, or in contrast, in cases where malignant cells have reverted to a post-EFT phenotype (AC cells) (surprisingly also known as what are commonly designated cancer stem cells), thereby becoming relatively resistant to apoptosis, the resistant “cancer stem cells” can be induced back to a pre-fetal phenotype to increase their susceptibility to treatments that induce apoptosis. These include the reprogramming of said AC cells using iTR reprogramming methods disclosed herein and previously disclosed in PCT Patent Application Ser. Nos. PCT/US2014/040601, PCT/US2017/036452, PCT/US2020/025512 and PCT/US2019/028816, each of which are incorporated in its entirety, (also known as the induction of senolysis of cancer stem cells (iS-CSC)), inhibiting the PI3K/AKT/mTOR (phosphoinositide 3-kinase/AKT/mammalian target of rapamycin) pathway such as with rapamycin or other inhibitors of mTOR, dietary restriction, or the use of dietary restriction mimetics. These and related uses of pathways related to the EFT in the diagnosis and treatment of cancer are the subject of the present invention.

### Example 1: Clustered Protocadherin Isoforms Differentially-Expressed in Embryonic Vs Fetal and Adult Non-Neuronal Somatic Cell Types

Pluripotent stem cell (PSC)-derived progenitors such as those derived clonally, display markers of primitive embryonic anlagen despite extensive passing or differentiation in vitro^{19, 20}. We therefore designated the lines “clonal embryonic progenitor cells” to distinguish them from fetal and adult cell counterparts. These cell lines represent primarily diverse stromal and parenchymal progenitors, as opposed to epithelial cell types, therefore we will designate the diverse clonal embryonic progenitor cell lines as “EPs” as compared to stromal fetal cells (“FCs”), and stromal adult non-epithelial lines (“ANE”). We utilized EPs and their fetal and adult cell type counterparts as an in vitro model of cells displaying a pre- and post EFT phenotype.

RNA-sequence data was obtained from diverse embryonic, adult, and cancer cell types including four different human ES cell lines and two iPSC lines (“PC” cells); 42 diverse clonal EP cell lines; eight FCs including three brown preadipocyte cultures and five fetal skin dermal fibroblasts spanning 8-16 weeks of development; 89 diverse stromal and parenchymal non-epithelial cell types (ANE), and five adult neuronal cell (NC) types including neurons and astrocytes. Since EP cells display a pre-fetal pattern of gene expression, we refer to them as “embryonic” contrasted with that of pluripotent stem cells (PCs) and data relating to diverse ANEs and adult epithelial cells (AECs) we designate “adult.”

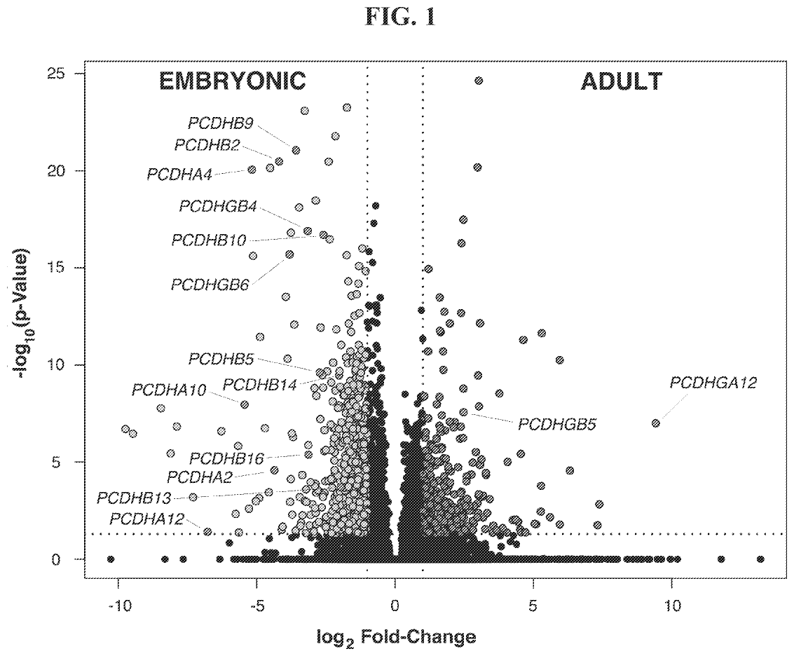

To identify global developmental alterations in gene expression that encompass numerous differentiated cell types that may account for the pre-EFT regenerative phenotype, we filtered the isoform data for highly statistically-significant gene expression differences distinguishing embryonic from adult cells regardless of differentiated cell type. 1079 isoforms showed a significant difference in expression (adjusted p value <0.05) in the EP vs ANE cells, with many of the most significant and largest fold-changes being isoforms of the CPL (FIG. 1) Embryonic up-regulated CPL isoforms were typically from the α and β clusters, while adult up-regulated isoforms were from the γ cluster. The γ isoforms with statistically-significant up-regulation in embryonic cells were PCDHA2, PCDHA4, PCDHA10, and PCDHA12. The γ isoforms with statistically-significant up-regulation in embryonic cells were PCDHB2, PCDHB5, PCDHB9, PCDHB10, PCDHB13, PCDHB14, and PCDHB16. The γ isoforms with statistically-significant up-regulation in embryonic cells were PCDHGB4 and PCDHGB5. In fetal and adult cells, all α and β isoforms were down-regulated while the γ isoforms with statistically-significant up-regulation in adult cells were PCDHGB6 and PCDHGA12.

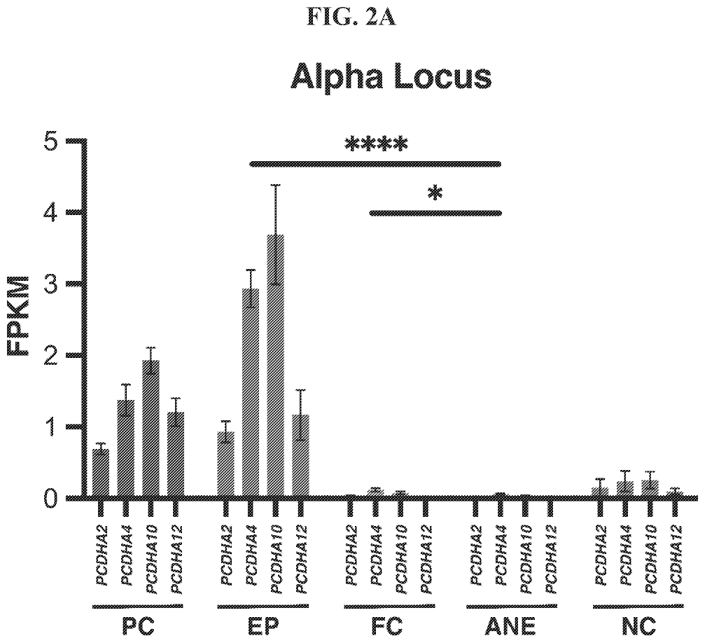

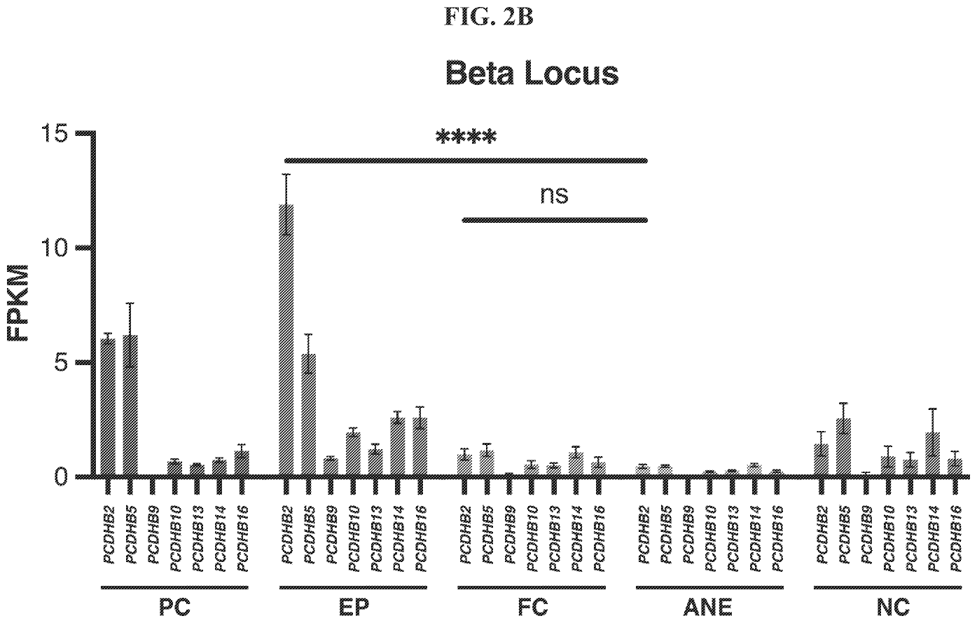

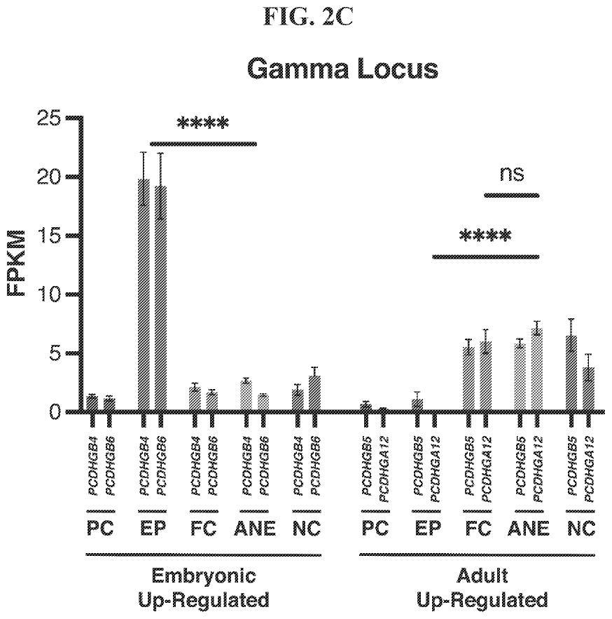

We chose for further study a representative member from each of the α and β loci (PCDHA4 and PCDHB2) that were significantly up-regulated in the majority of embryonic cells compared to adult counterparts (p-values of 8.8×10⁻²¹ and 3.3×10⁻²¹ respectively), and showed mean fold-changes of embryonic versus adult expression (FPKM) of 36.1 and 18.2 respectively (FIGS. 2A-2C). From the γ locus, the isoform PCDHGA12 showed adult up-regulation in the majority of adult cell types with an adjusted p-value of 1.0×10⁻⁷ and a mean fold-change in adult versus embryonic expression (FPKM) of 686-fold.

### Example 2: The Onset of Adult CPL Isoform Expression Occurs at or Before the Embryonic-Fetal Transition

The EFT is commonly associated with a loss of the capacity for scarless regeneration in numerous tissues of the mammalian body. In the case of human skin, this appears to coincide approximately with Carnegie stage 23 (eight weeks of gestation). We therefore tested the hypothesis that the transition from an embryonic scarless regenerative phenotype to a fetal/adult non-regenerative state correlates temporally with altered CPL isoform expression. We examined the expression of CPL isoforms in early passage fibroblasts from the medial aspect of the upper arm synchronized in quiescence (Fetal Cells (FCs)) in comparison with EP and ANE counterparts (FIGS. 2A-2C). In the case of the representative genes PCDHA4, PCDHB2, and PCDHGA12, CPL isoform expression in 8-16 week-old FCs appeared exhibited an adult-like pattern of expression with the possible exception of PCDHA4 where FCs showed a modestly-significant elevation over adult counterparts (ANE cells) (p<0.05). A broader analysis of 34 early passage dermal fibroblasts from adults aged 11-83 years all showed an adult pattern (low PCDHA4, low PCDHB2, and high PCDHGA12 with no significant alteration with during aging in vivo. We conclude that the transition in CPL expression may therefore occur before the EFT (Human Carnegie stage 23) in vivo.

PC cells such as embryonic stem (ES) and induced pluripotent stem (iPS) appeared to display a subset of isoforms including PCDHA2, PCDHA4, PCDHA10, PCDHA12, PCDHB2, PCDHB5, and like EPs, express low levels of the adult markers PCDHGB5 and PCDHGA12 (FIG. 2). Importantly, the expression level of the CPL isoforms up-regulated in embryonic cells (both PCs and EPs) was comparable to the CNS-derived cell types which included neurons and astrocytes of diverse origin. Given the evidence in support of the role of CPL isoforms in the development of the CNS, the comparable or even greater levels of transcripts for CPL isoforms in diverse somatic cell types outside the CNS in EPs is consistent with the critical role that CPL isoforms are expressed in the embryonic (pre-EFT) state, and may therefore play a role in cell-cell recognition during development.

### Example 3: Diverse Cancer Cell Lines Commonly Display an Embryonic Pattern of CPL Isoform Expression (Embryo-Onco Phenotype)

Shared gene expression changes spanning diverse somatic cell types subsequent to embryonic organogenesis may reflect antagonistic pleiotropy. That is, the repression of pathways critical for cell-cell recognition and morphogenesis may limit regeneration, but have the selective advantage of providing effective tumor suppression. We previously demonstrated such a role for the catalytic component of telomerase (TERT) by screening for evidence of repression across diverse normal somatic cell types together with their malignant counterparts 8. We therefore performed a similar survey using RNA-seq-based transcriptomic data of the previously-described PCs, diverse EPs, diverse ANE cell types as well as 24 diverse normal human adult epithelial cell (AEC) types, 39 diverse human sarcoma cell (SC) lines including cells derived from all three germ layers including normal counterparts to many sarcomas and carcinomas, and 35 diverse carcinoma and adenocarcinoma cell (CAC) lines.

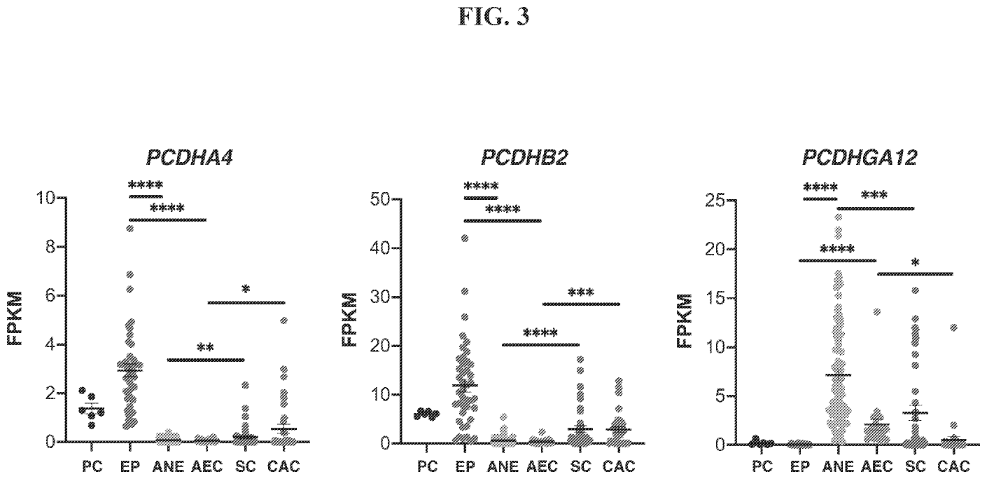

As shown in FIG. 3, the representative embryonic CPL isoform transcript markers PCDHA4 and PCDHB2 are significantly down-regulated in ANE cells and AECs compared to EPs (p<0.0001). Similarly, PCDHGA12 expression was significantly elevated in ANE cells and AECs compared to EPs (p<0.0001). Interestingly, diverse cancer lines such as SCs and CACs (representing cancers originating from stromal and epithelial cell types respectively) when compared to normal ANE and AEC cultures respectively showed a highly significant shift toward an embryonic pattern of CPL gene expression. In the case of the c loci, all α isoforms (PCDHA1-13) as well as PCDHAC1 and PCDHAC2 were up-regulated in diverse SCs and CACs including marked up-regulation in neuroblastomas, glioblastomas, sarcomas including uterine, synovial, rhabdomyosarcomas, renal rhabdomyosarcomas, Ewings sarcomas, chondrosarcomas, osteosarcomas, bone giant cell sarcomas, leiomyosarcomas, liposarcomas; carcinomas including breast ductal, and bronchioalveolar carcinomas; and breast adenocarcinomas; and B-cell lymphoblastic leukemias compared to adult counterparts.

α and β loci, PCDHA4 and PCDHB2 are significantly up-regulated in SCs and CACs compared to normal ANE and AEC counterparts (p<0.01 and p<0.05 respectively in the case of PCDHA4 and p<0.0001 and p<0.001 respectively in the case of PCDHB2). In the case of the β loci, all β isoforms (PCDHB1-6 and PCDHB8-16) as well as PCDHB17P, PCDHB18P and PCDHB19P were up-regulated in diverse SCs and CACs including marked up-regulation in neuroblastomas, glioblastomas, sarcomas including pagetoid, uterine, synovial, rhabdomyosarcomas, renal rhabdomyosarcomas, Ewings sarcomas, chondrosarcomas, osteosarcomas, leiomyosarcomas, bone giant cell sarcomas, liposarcomas; carcinomas including breast, ductal, endometrial, hepatocellular, and bronchioalveolar carcinomas; prostate and breast adenocarcinomas; and B-cell lymphoblastic leukemias compared to adult counterparts.

Similarly, PCDHGA12 expression was significantly reduced showing a more embryonic pattern in SCs and CACs (p<0.001 for SCs compared to normal ANE counterparts and p<0.05 for CACs compared to normal AEC counterparts). In the case of the γ loci, PCDHGB5 and/or PCDHGA12 were down-regulated in diverse SCs and CACs including marked down-regulation in neuroblastomas, glioblastomas, sarcomas including uterine, pagetoid, synovial, muscle rhabdomyosarcomas, renal rhabdomyosarcomas, Ewings sarcomas, Wilm's tumor, chondrosarcomas, fibrosarcomas, osteosarcomas, bone giant cell sarcomas, leiomyosarcomas, liposarcomas; carcinomas including squamous cell, epidermoid, hepatocellular, breast ductal, prostate, renal, and bronchioalveolar carcinomas; colorectal, and breast adenocarcinomas; and B-cell lymphoblastic leukemias compared to adult counterparts. Unexpectedly, PCDHGB4 and PCDHGB6 were not elevated in expression in SCs and CACs despite the fact that they are up-regulated in embryonic cells. Therefore, when the present invention refers to pre-cancer or cancer cells expressing an embryonic pattern of CPL isoform expression, we are not referring to PCDHGB4 and PCDHGB6.

Similarly, RNA-seq analysis showed expression of one or more of the α loci, isoforms (PCDHA1-13) as well as PCDHAC1 and PCDHAC2 in lung, esophageal, ovarian, endometrial, pancreatic, anaplastic large cell lymphoma, urinary tract, autonomic ganglial, Burkitt lymphoma, biliary tract, acute lymphoblastic T-cell leukemia, melanoma, diffuse large B-cell lymphoma, plasma cell myeloma, blast phase chronic myeloid leukemia, acute myeloid leukemia, Hodgkin lymphoma, small lymphocytic lymphoma, and mantle cell lymphoma cancer cells, whereas their normal adult counterparts expressed low to no levels of the transcripts.

Similarly, RNA-seq analysis showed expression of one or more of the β loci, (PCDHB1-6 and PCDHB8-16) as well as PCDHB17P, PCDHB18P and PCDHB19P in lung, plasma cell myeloma, melanoma, pleural, ovarian, acute myeloid leukemia, endometrial, renal, Burkitt lymphoma, acute lymphoblastic T-cell leukemia, liver, astrocytoma, esophageal, pancreatic, gastric, plasma cell myeloma, Hodgkin lymphoma, autonomic ganglia, anaplastic large cell lymphoma, biliary tract, B-cell lymphoma, adult T-cell lymphoma, essential thrombocythemia, acute myeloid leukemia, and blast phase chronic myeloid leukemia whereas normal counterparts did not express said isoforms.

Similarly, RNA-seq analysis showed low to no expression of one or both of the γ cluster isoforms PCDHGB5 and/or PCDHGA12 in lung, stomach, urinary tract, esophageal, pancreatic, ovarian, biliary tract, pleural, thyroid, salivary gland, whereas normal counterparts expressed the gene. Furthermore, expression of one or both of the γ cluster isoforms PCDHGB5 and/or PCDHGA12 was markedly higher in the hematopoietic or lymphoid cancer lines compared to their normal blood cell counterparts.

While both sarcoma and carcinoma cell lines appear to frequently display an embryonic pattern of CPL gene expression (i.e. up-regulated PCDHA4 and PCDHB2 and down-regulated PCDHGA12), the combination of individual isoform expression in the lines appear heterogeneous. This is consistent with the hypothesis that CPL isoforms play a dual role in both differentiated cell type-specific cell-cell recognition as well as regulating alterations unique to the EFT. In addition, sarcoma cell lines appeared heterogeneous in regard to their embryonic vs adult-like CPL isoform profile, carcinoma more frequently embryonic. In the case of the marker PCDHGA12, the majority of normal adult cells expressed the isoform while the majority of cancer cell lines did not. For example, using the cutoff of 0.5 FPKM as a lower limit of expression, only 2/97 adult-derived stromal and parenchymal cell types (hepatocytes in both cases), and 1/23 cultured epithelial cell types did not express PCDHGA12. However, 21/39 (54%) of sarcoma lines and 33/35 (94%) of carcinoma and adenocarcinoma cell lines did not express PCDHGA12 at a level of at least 0.5 FPKM. In a manner unique to cancer cell lines, 6/39 (15%) of sarcoma and 5/35 (14%) of carcinoma lines expressed PCDHA1 at a level >0.5 FPKM, while no PCs, EPs, FCs, ANEs, or BCs expressed the gene at that threshold level.

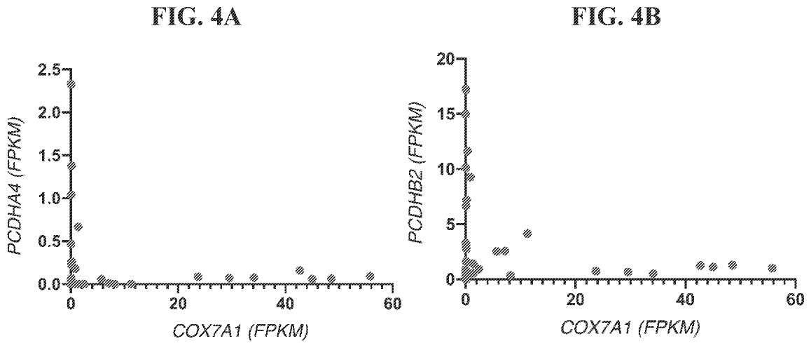

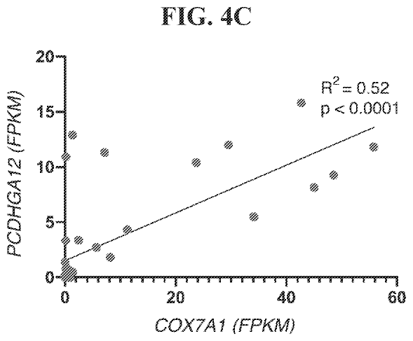

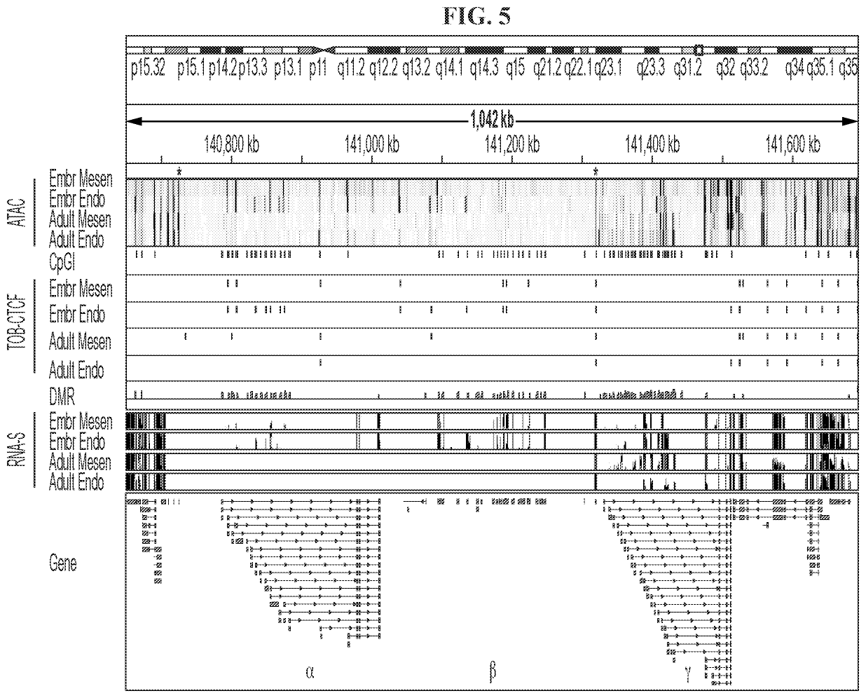

To determine whether the embryo-onco phenotype extends to genes other than those of the CPL, we examined the correlation of PCDHA4, PCDHB2, and PCDHGA12 expression with COX7A1, previously reported to be a robust marker of the EFT in many stromal and parenchymal cell types¹⁹. COX7A1 expression commences at approximately the EFT, plateauing in adulthood, and is not expressed in the majority of cancer cell lines¹⁹. As shown in FIG. 4, the adult marker COX7A1 appears to be inversely correlated with the embryonic markers PCDHA4 and PCDHB2 while showing a trend toward directly correlating with the expression of the adult marker PCDHGA12 in diverse sarcoma cell lines (R²=0.52, p<0.0001). These data therefore suggest that the majority of sarcoma, carcinoma, and adenocarcinoma cell lines display an embryonic pattern of CPL isoform expression, correlating with a down-regulation of COX7A1 in support of an embryo-onco phenotype inclusive of the altered expression of multiple genes.

### Example 4: Diagnosis of CPL Isoform Status in Blood Cells and Related Therapeutic Methods

As disclosed in Example 3 supra, blood cell cancers, especially lymphoid cancers, surprisingly abnormally express embryonic and/or fetal marker CPL isoforms. One skilled in the art will recognize that this observation leads to a number of novel diagnostic and therapeutic strategies to treat patients with these cancers.

The diagnosis of the CPL isoforms expressed by tumor cells can be determined by RNA, DNA, or protein-based assays. In the case of RNA assays, by way of nonlimiting examples, the transcriptome of tumor cells from a patient can be measured using gene expression microarrays, PCR, or RNA-sequencing.

As an example of the determination of CPL c isoform expression in hematopoietic and lymphoid cancers, RNA-sequence data from 170 blood cancer cell lines showed that acute myeloid leukemia, blast phase chronic myeloid leukemia, mantle cell lymphoma, Hodgkin lymphoma, plasma cell myeloma, and diffuse large B-cell lymphoma commonly showed abnormally high levels of LMNA and PCDHA12 expression characteristic of an adult CPL isoform pattern of expression. Therefore, PCDHGA12 antigen is a novel target for dendritic cell or CAR-T cell based therapeutic strategies.

### Example 5: Altered Embryonic Vs Adult Gene Expression Coincides with Modifications in Chromatin Accessibility, CTCF Binding, and Hypermethylated CpG Islands

We next examined the epigenetic status of the α, β, and γ loci beginning with ATAC (Assay of Transposase Accessible Chromatin) sequencing to identify accessible regions of chromatin and potential interactions with DNA binding proteins²¹. Accessibility coincided with expressed CPL genes in embryonic and adult counterparts of osteogenic mesenchyme and vascular endothelium as determined by the mRNA read coverage (FIG. 5). ATAC accessibility also frequently coincided with CTCF footprints as determined by TOBIAS. CTCF has previously been reported to participate in gene regulation in the CPL by regulating the structure of chromatin domains and regulating cis interactions with enhancers²².

We next examined CpG methylation in the α, β, and γ loci as determined by Whole Genome Bisulfite Sequencing (WGBS). Four hES cell-derived clonal EP cell lines were sequenced together with their respective adult-derived counterparts, namely: 4D20.8, a clonal embryonic osteochondral progenitor²³ and adult bone marrow-derived mesenchymal stem cells (MSCs); 30-MV2-6, a clonal embryonic vascular endothelial cell line together with adult-derived aortic endothelial cells (HAECs); SK5 a clonal embryonic skeletal muscle progenitor together with adult skeletal myoblasts; and E3, an embryonic white preadipocyte cell line²⁴ together with adult subcutaneous white preadipocytes. Differentially-methylated regions (DMRs) were identified based on significant (p<0.05) difference in methylated CpGs in three of four of the EP/ANE pairs using Metilene software. DMRs in the CPL are shown in FIG. 5 as previously described (see PCT Patent Application Ser. No. PCT/US2020/047707 and U.S. patent application Ser. No. 17/251,145, each of which are incorporated in its entirety). DMRs co-localized with the first exon of α and γ isoforms and the gene body of β isoforms, with CpG islands (CpGI), the pattern of read coverage, and were uniformly hypermethylated in embryonic cells compared to adult counterparts regardless of whether the isoform was embryonic or adult-specific. Of the 21,317 significant DMRs identified in the entire genome, the majority (20,022) were hypermethylated (data not shown), however, overall genomic CpG methylation was not significantly different in EPs vs their adult counterparts.

### Example 6: Embryonic DMRs within the CPL are Observed in Diverse Cancer Cell Lines and Appear Distinct from the CpG Island Methylator Phenotype (CIMP)

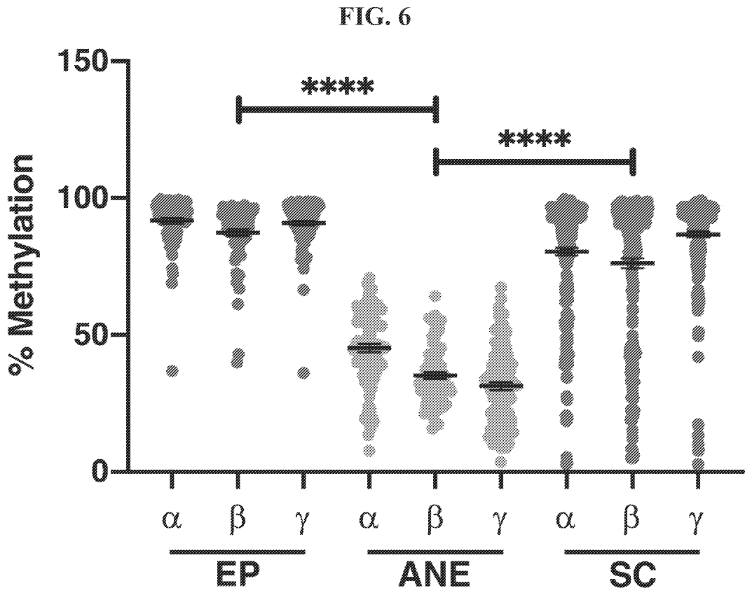

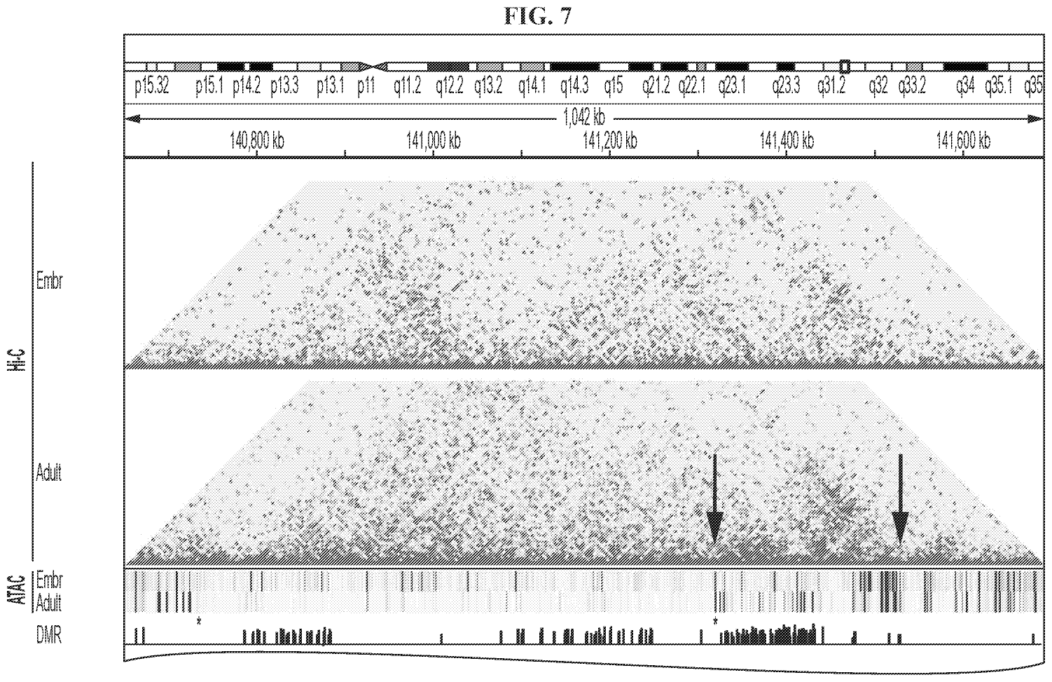

We next determined DMR methylation status in sarcoma cell (SC) lines from cell types corresponding to the EPs and ACs of mesenchymal, endothelial, myoblast, and preadipocyte cell differentiated states. The results are summarized in FIG. 6 for the genes PCDHA4, PCDHB2, and PCDHGA12. In the case of PCDHA4 and PCDHB2, the differences in percent methylation between embryonic and adult cells was highly significant (p<0.0001). In the case of PCDHA4 and PCDHB2, DMRs tended to be associated with gene bodies rather than promoter regions. The DMR associated with the adult-onset gene PCDHGA12 overlapped with the promoter of the gene, and was significantly (p<0.001) hypermethylated in embryonic cells, despite the accessibility and expression of the gene being adult onset. We therefore conclude that the sarcoma cell lines studied exhibited a DMR pattern more closely resembling an embryonic pattern of methylation than that of their adult counterparts (FIG. 6).

The CpG Island Methylator Phenotype (CIMP) is a commonly-studied category of DMRs hypermethylated in cancer²⁵. A panel of CIMP DMRs markers commonly used in colorectal cancer include those associated with CACNA1G, CDKN2A, CRABP1, IGF2, MLH1, NEUROG1, RUNX3, and SOCS1 genes²⁶. While these DMRs within CpGIs show hypermethylation in a number of the cancer cell lines, they do not appear to be significantly (q-value <0.05) differentially-methylated in embryonic vs adult cell types. The IGF2 and RUNX3 CpGIs were an exception, however they are significantly hypermethylated in adult cells, in contract to the DMRs in the CPL locus. Therefore, we conclude that the DMRs associated with the embryo-onco phenotype in the CPL do not reflect the CIMP as it is commonly described, but instead may reflect an embryo-specific methylator phenotype which we refer to herein as CIMP-E.

### Example 7: Chromatin Architecture in the CPL Appears Altered in the Embryonic Vs Adult Cell Types in Alignment with Lamina-Associated Domains

As shown in FIG. 5, the ˜0.6 MB region spanning the ca and R clusters (marked with asterisks) displayed markedly less accessibility in both adult osteogenic mesenchymal cells (MSCs) and adult aortic endothelial cell lines compared to their embryonic counterparts. Lamin interactions, such as those associated with lamin A/C and lamin B1 can impart heterochromatic alterations in chromatin spanning >0.1 megabases associated with the nuclear periphery (lamin-associated domains (LADs)), we therefore examined the association of LADs with the CPL locus. Recognizing that lamin A appears to have dual roles at the nuclear periphery (isolated by sonication), and intranuclear (isolated by micrococcal nuclease digestion), we utilized previously identified lamina-interacting domains (LiDs) associated with lamin B1, and lamin A in HeLa cells from both sonicated and micrococcal nuclease-prepared samples assayed by ChIP-seq²⁷ as a more comprehensive survey of chromatin interactions with lamins.

As shown in FIG. 7, the lamin B1 LAD co-localized closely with the inaccessible region of the CPL locus in adult cells and was demarcated by CTCF binding sites²⁸. Furthermore, the micrococcal nuclease-prepared Lamin A ChIP-seq sample appeared to closely overlap the region associated with Lamin B1 but to also extend over the γ locus and a 3′ superenhancer region identified using dbSuper²⁹.

LADs have commonly been associated with peripheral heterochromatin marked by H3K9me3, demarcated by CTCF binding sites which are thought to play a role in recruiting transcription factors as well as functioning as insulators in defined topological domains thereby regulating the function of enhancers, and for instituting a repressive environment for gene expression²⁸ 30. Consistent with these markers of LADs, we observed a pronounced island of the heterochromatic marker H3K9me3 as well as H4K20me3 overlapping with the CPL LAD in both embryonic and adult cells. Additionally, H3K27me3 marks were observed to be markedly higher in the γ locus in the region of MNase-derived (presumably subnuclear (nucleoplasmic) as opposed to the peripheral inner nuclear membrane-associated) lamin A-specific binding. H3K27me3, a product of the EZH2 methyltransferase, like H3K9me3 and H4K20me3, is generally considered to repress gene expression. However, as described above, the CPL isoforms are nevertheless expressed in diverse embryonic and adult cell types despite these heterochromatic marks. In addition, unlike the generally-accepted belief that H3K4me3 marks are reduced in the repressive environment of a LAD²⁸, we observed strong H3K4me3, as well as H3K27Ac, and H3K9Ac marks in association with all the expressed isoforms with the α, β, and γ loci (FIG. 7). H3K27Ac and H3K9Ac, like H3K4me3 are considered markers of active gene expression³¹, consistent with the pattern of gene expression noted above.

ATAC-Seq showed open footprints for CTCF binding sites as determined by TOBIAS in regions other than those associated with CPL CpGIs (FIG. 7, marked with black arrows). The most 3′ of these two CTCF sites is outside of the γ cluster and resides within a superenhancer region. The presence of CTCF binding at these two sites as determined by TOBIAS in all cells assayed (FIG. 5), suggests that a potential topological domain exists in both embryonic and adult cells at this location. To confirm this potential constitutive topological domain, we utilized chromosome conformation capture (Hi-C) to reconstruct potential pairwise interactions based on domain topology. As shown in FIG. 7, both embryonic and adult cells appear to show an interaction between the aforementioned twin CTCF sites (marked by the black arrows).

### Example 8: Homologous Expression of CPL Isoforms in Varied Types of Differentiated Cells and Anatomical Locations

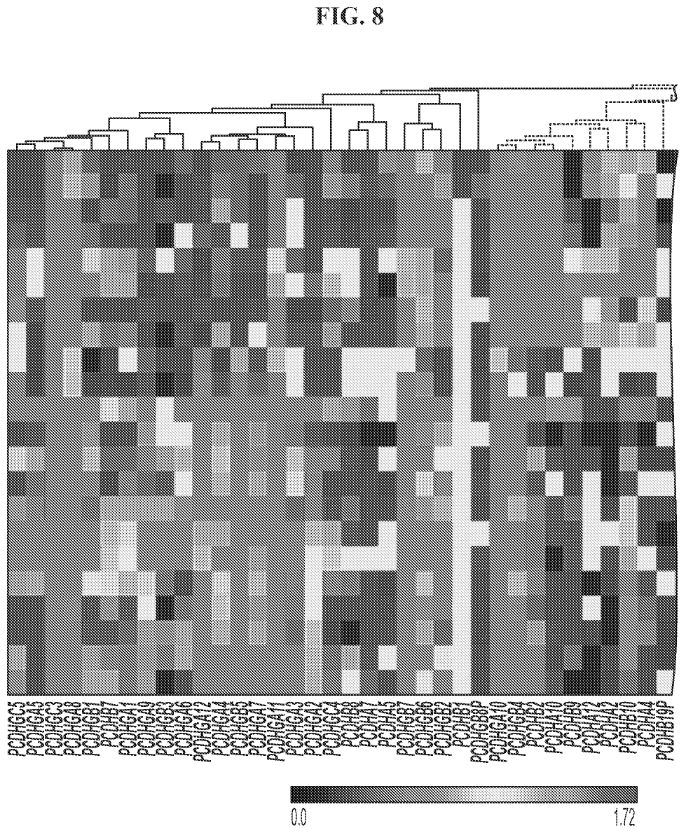

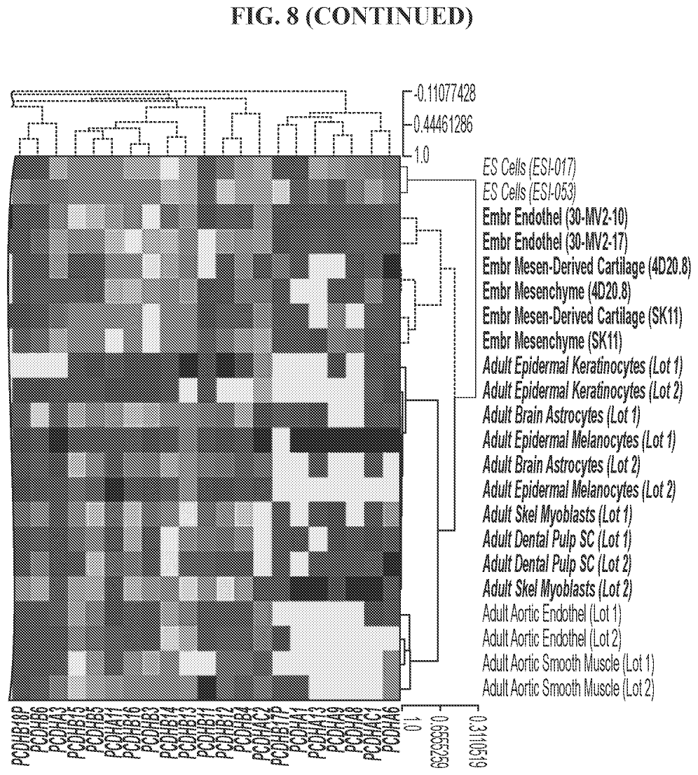

Homologous expression of CPL isoforms has been previously demonstrated to lead to cell-cell aggregation^{13, 32}. While CPL isoforms are reported to be expressed stochastically from each allele in neuronal cells, presumably to regulate self-recognition³³, patterns CPL isoforms would be expected to be uniquely and uniformly expressed within particular differentiated cell types if they were to form homophilic interactions with their ectodomains and thereby play a role in embryonic tissue morphogenesis. We tested whether our data was consistent with this hypothesis in silico by examining the pattern of CPL isoform expression in biological replicates of particular differentiated cell types with hierarchical clustering objectively determining expression homology. We therefore performed a hierarchical clustering of a subset of the EP and ACs. As shown in FIG. 8, ES cells (ESI-017 and ESI-053) clustered together but with the greatest divergence from the diverse differentiated cell types. The next layer of clustering effectively differentiated EP and ANE cells as predicted, since the basis of this study was their differential expression of α, β, and γ isoforms between these two categories of cells. Most significantly, replicates of embryonic ES-derived vascular endothelium (30-MV2-10 and 30-MV2-17) clustered closely together. Similarly, ES-derived progenitors of cartilage (4D20.8 and SK11)³⁴, and resulting chondrocytes clustered closely together. In the case of adult cells, ectodermally-derived cell types such as keratinocytes, melanocytes, and astrocytes clustered closely as did dental pulp and skeletal myoblasts. Interestingly, Adult aortic endothelial and adult aortic smooth muscle cells reproducibly clustered together, consistent with their anatomical colocation and capacity for self-assembly³⁵. Therefore, this in silico modelling is consistent with the hypothesis that combinations of CPL isoform expression in diverse somatic cell types may uniquely profile them, potentially playing a role in the adhesion of similar cells, or their adhesion to anatomically-related cells.

### Example 8: CPL γ Isoforms are Down-Regulated During Cell Senescence in Vitro

The alterations we observed in α, β, and γ isoform gene expression in the course of embryonic-fetal development may reflect evolutionary selection for tumor suppression once embryonic organogenesis is complete. Since telomerase repression early in embryonic development leading to subsequent somatic cell replicative mortality is believed to reflect a similar example of antagonistic pleiotropy, we asked whether there are any alterations in CPL isoform expression occur during the aging of dermal fibroblasts in vivo and in vitro.

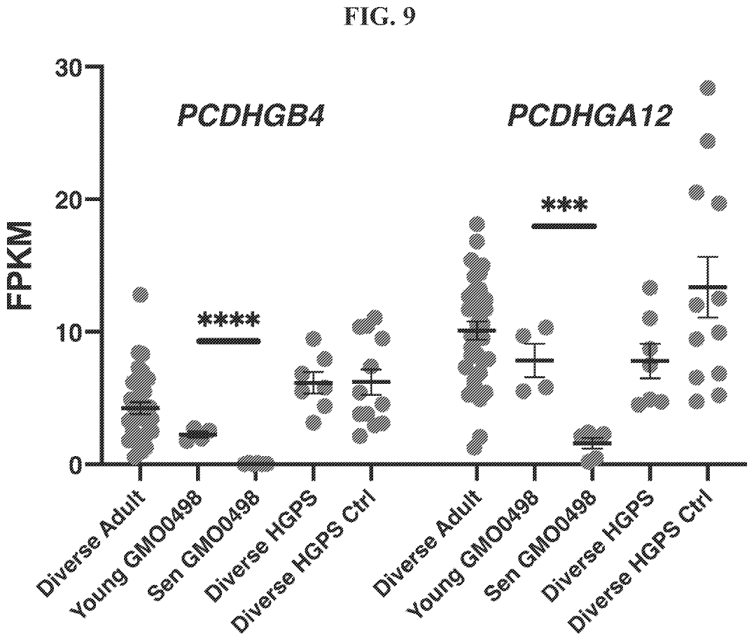

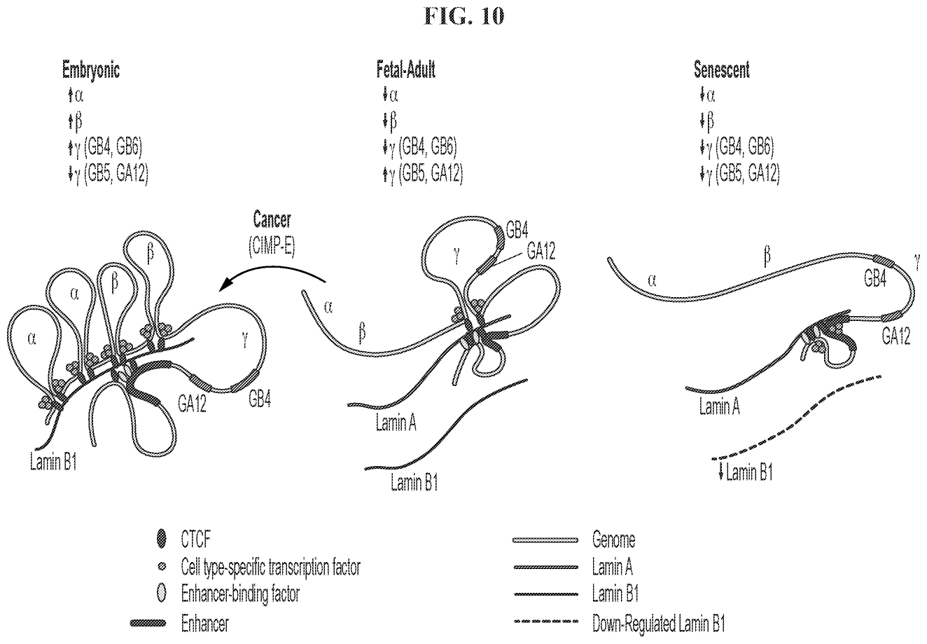

The γ isoform that was down-regulated during the EFT such as PCDHGB4, or up-regulated during EFT (such as PCDHGA12), and indeed all γ isoforms showed down-regulation during senescence in vitro. In regard to in vivo aging, significant up-regulation of γ isoforms including PCDHGB4 and PCDHGA12 were observed in postnatal aged fibroblasts compared to fetal (synchronized dermal fibroblast lines from the medial aspect of the upper arm aged 11-83 years and 8-16 weeks gestation respectively). However, no significant trends were observed during aging in vivo in the post-natal period. In addition, dermal fibroblasts derived from the Hutchinson-Gilford Progeria Syndrome (HGPS) showed no significant differences compared to 12 age-matched control lines. However, as shown in FIG. 9, isoforms in the γ locus decreased significantly with replicative senescence in vitro. Culture of cells in log growth in 10% serum as opposed to quiescence in 0.5% serum appeared to increase PCDHGA12 expression, highlighting the importance of growth and culture synchronization in expression analysis of CPL isoforms.

### Example 9: Lamin and CPL Isoform Expression in Development, Aging, and Cancer

We next asked whether CPL isoform expression correlated with that of lamin A or lamin B1 during development, aging, or cancer. As previously reported³⁶, we observed that pluripotent stem cells expressed markedly lower levels of LMNA, while relatively high levels of LMNB1 compared to fetal or adult counterparts. In regard to in vitro senescence, we again observed serum and/or growth-related effects on expression of both lamins. When comparing early passage (P19) dermal fibroblasts with senescent counterparts (P38), we saw a significant increase in LMNA and a significant decrease in LMNB1 as previously reported³⁷.

In the case of the diverse sarcoma and carcinoma cell lines used in this study, we saw a significant correlation of increasing expression of LMNA and decreasing expression of LMNB1 correlating with PCDHGA12 expression in the cancer cell lines. The role of lamins A and B1 in organizing expression domains leads to the intriguing question as to whether they play causative role in the regulation of altered gene expression in the embryonic-fetal transition during normal development and cancer or are downstream of other yet unknown regulatory events.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

### Example 10: Targeting Embryonic (Pre-Fetal) CPL Isoforms on Cancer Cells with Members of the Immunoglobulin Superfamily to Induce Cell Death

The surprising expression of members of the alpha and beta CPL isoforms in embryonic (prefetal, or pre-EFT) cells and diverse cancer cell types, but not most adult cell types with the exception of CNS cells, offers the opportunity to facilitate an immunological attack on said members of the alpha and betal CPL isoforms as a therapeutic strategy for diverse type of cancer. We first confirmed that embryonic (pre-fetal)-specific CPL isoforms such as PCDHB2 and PCDHB2 were expressed on a protein level similar to mRNA levels. The hESC-derived clonal embryonic (pre-fetal) progenitor cell lines 4D20.8 (osteochondral progenitor) and 30-MV2-6 (embryonic vascular endothelium) showed protein intensities of 3301 and 6807 respectively compared to the protein intensities in normal human aortic endothelial cells and mesenchymal stem cell counterparts (protein intensities of 1 and 73 respectively). The PCDHB2 and PCDHB3 proteins were the most significantly-differentially-expressed proteins measured out of 7418 proteins assayed by mass spectrometry. Therefore, since CPL proteins are expressed on the plasma membrane and exposed extracellularly, they may be targeted by members of the immunoglobulin superfamily including monoclonal or polyclonal antibodies such as IgG or IgM with specific affinity to the members of the alpha or beta CPL isoforms, or said antibodies conjugated to toxins or imaging ligards for diagnostic purposes, or bi-specific antibodies such as bi-specific T-cell engagers composed, by way of nonlimiting example, of two single-chain variable fragments wherein one variable fragment binds to the target alpha or beta CPL isoform and the other to a T-Cell antigen such as CD3.

Two types of cancer cells that express embryonic (pre-fetal) CPL isoforms were examined to test the ability of immunoglobulin to target and facilitate lysis specifically of adult-derived human cancer cells, but not normal adult human cells. The breast cancer cell line BT-20 which expressed 8.57 FPKM of PCDHB3 transcript and the lung cancer cell line NCI-H358 which expressed 6.56 FPKM of PCDHA3 transcript and normal human fibroblasts (MDW-1) which did not express alpha or beta CPL isoforms were examined. Cells were exposed to rabbit polyclonal anti-sera specific to CPL isoforms expressed on the cancer cells or isotype antibody control. Cells were then counted after exposure to the specific polyclonal Abs against embryonic PCDHs on cancer lines and rabbit PBMCs containing CD8 T-cells and natural killer cells.

Two human carcinoma cell lines (1) BT-20 (ATCC HTB-19 mammary gland carcinoma, epithelial) and (2) NCI-H358 (ATCC CRL-5807 bronchioalveolar carcinoma, non-small cell) and one dermal fibroblast line MDW-1 (AgeX Therapeutics, Alameda) were cultured in T-175 flasks with DMEM plus glutamax and 10% FBS in a humidified incubator at 37° C. and 5% CO₂.

The cells of each line were detached, counted, and evenly seeded at 25,000 cells into 24 well plates.

Next day, after attachment, the cells were treated with individual Abs (0.1 mg/ml, 100 ul, polyclonal rabbit IgG anti-human from ThermoFisher, (Cat #s PA5-119380, 101272-2-AP, PA5-31297, PA5-110079, PA5-31129, BS-13726R, PA5-63484), or IgG isotype control (Cat #02-6102) and Ab combinations. Each cancer cell line was exposed to Abs based on their expression of PCDH transcript which was determined previously. Also, a normal adult-derived dermal fibroblast line (MDW-1) was treated similarly with each Ab and each Ab combination used on the cancer lines as an additional control.

To 250 ul medium (DMEM+10% FBS) per well 2 ul and 4 ul of individual Abs (representing Ab dilutions of 125 and 62.5 respectively), as well as the Ab combinations (at 2 ul and 4 ul per Ab), were added to each well.

After addition of the Abs, the plates were placed in a humidified incubator at 37° C. with ambient O₂ and 5% CO₂ for 90 minutes. Then, the wells were washed 3× with PBS (containing Ca and Mg) to remove unbound Ab.

In 250 ul medium volume 10 ul of rabbit PBMC (SKU: IQB-RbPB102, IQ Biosciences, Berkeley CA) at 5.0×10e6 cells per ml was added to each well and placed in the incubator for 24 hours. The medium containing PBMC was removed and the wells were washed 3× with PBS.

To examine whether there was any loss of cells or cell lysis, the cells were stained with Live/Dead viability/cytotoxicity kit (ThermoFisher Cat. V13154) agents Calcein AM at 2.0 uM and 4 uM ethidium bromide in 20 mM HEPES buffered HANKS balanced salt medium for 45 min in a humidified incubator at 37° C., 5% CO₂. The wells were then rinsed to remove free dyes and fresh growth medium was added.

After staining, the cells were visualized using an EVOS cell imaging system equipped with fluorescence (Invitrogen). Lastly, total cell counts per well were obtained following detachment of cells (75 ul TrypLE and 225 ul medium) using a ThermoFisher Countess cell counter.

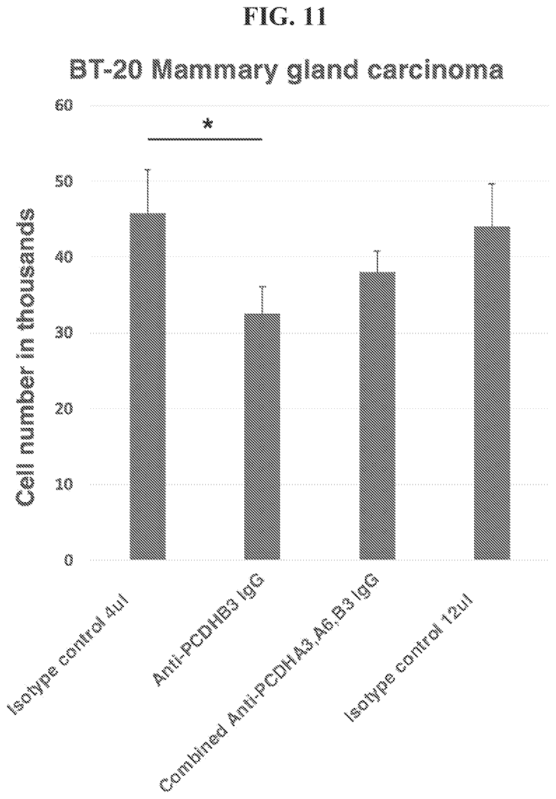

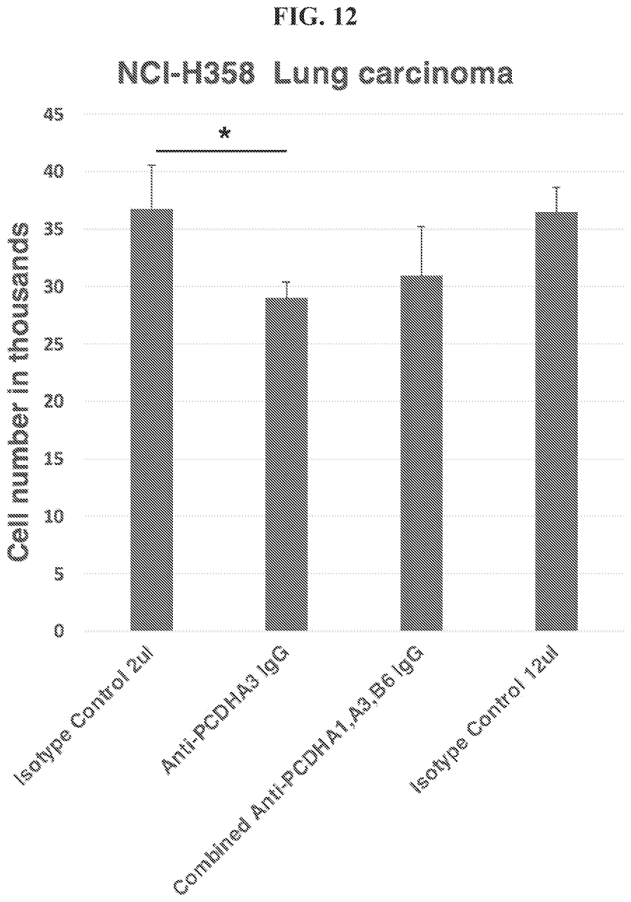

Total cell counts were taken of each well to assess any cell loss or lysis. With both cancer lines there was statistically-significant (p<0.05) fewer remaining cells in the PCDH Ab treated wells, which was most pronounced at the higher Ab concentration as might be expected. FIGS. 11 and 12 show the cell counts in breast cancer and lung cancer with antibody to the CPL isoforms shown together with isotype antobody. In particular, the greatest loss of cells was observed when PCDHB3 Ab was used to treat BT-20 HTB-19 (mammary gland carcinoma, epithelial). In this case the number of cells remaining using PCDHB3 Ab at 2.0 ul and 4.0 ul were (41,500 and 32,500) compared to isotype control treatment (46,250 and 45,750), and as expected, was lowest at the higher Ab concentration. Consistent with this observation, the antibody combination (PCDHA3, PCDHA6, and PCDHB3) which includes PCDHB3, at both Ab concentrations tested, had a trend toward fewer cells on average (39,500 and 38,000) than isotype controls (43,000 and 44,000 respectively).

Similarly, treatment with expressed PCDH antibodies (including PCDHA1, A3 and B6) of the bronchioalveolar cancer cell line NCI-H358 demonstrated statistically-significantly fewer remaining cells (PC 0.05) compared to isotype treated controls. The most striking data on the lung cancer line was obtained using PCDHA3 antibody which showed fewer cells 32,000 and 29,000, at the lower and higher Ab concentration respectively, compared to isotype control 36,750 and 37,000 respectively. As with the mammary cancer line the antibody combination treatment that included PCDHA3 (i.e. PCDHA1, A3, B6) also had a trend toward fewer cells, 32,000 and 31,000, compared to isotype control, 35,000 and 36,500.

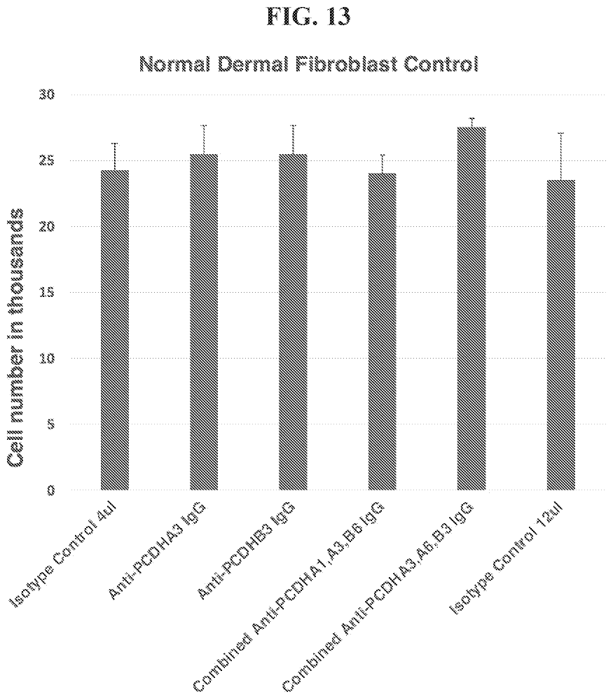

Lastly, the fibroblast line control, treated with all the same PCDH Abs and Ab combinations used in the cancer lines, displayed consistent cell numbers in all wells, as expected, since they lack the embryonic surface PCDH antigens which the Abs interact (FIG. 13).

Therefore, CPL isoforms are expressed on a protein level, and can be targeted with immunoglobulin superfamily members such as antibody-based therapies or T-cell receptor strategies such as CAR-T cells. Furthermore, use of said immunoglobulin superfamily members have the potential of targeting cancer cells while sparing normal adult human tissues, with the possible exception of CNS cells.

### INCORPORATION BY REFERENCE

All publications and patents cited in this specification are herein incorporated by reference as if each individual publication or patent were specifically and individually indicated to be incorporated by reference and are incorporated herein by reference in their entirety to disclose and describe the methods and/or materials in connection with which the publications are cited. The citation of any publication is for its disclosure prior to the filing date and should not be construed as an admission that the present invention is not entitled to antedate such publication by virtue of prior invention.

The following references, to the extent that they provide exemplary procedural or other details supplementary to those set forth herein, are specifically incorporated herein by reference.

Although illustrative embodiments of the present invention have been described herein, it should be understood that the invention is not limited to those described, and that various other changes or modifications may be made by one skilled in the art without departing from the scope or spirit of the invention.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### REFERENCES

1. Grunwald, G. B. The conceptual and experimental foundations of vertebrate embryonic cell adhesion research. Dev Biol (N Y 1985) 7, 129-158 (1991).
2. Steinberg, M. S. & Gilbert, S. F. Townes and Holtfreter (1955): directed movements and selective adhesion of embryonic amphibian cells. J Exp Zool A Comp Exp Biol 301, 701-706 (2004).
3. Wilson, H. V. On Some Phenomena of Coalescence and Regeneration in Sponges. J. Exp. Zool. 5, 245-258 (1907).
4. Spemann, H. & Mangold, H. Induction of embryonic primordia by implantation of organizers from a different species. 1923. Int J Dev Biol 45, 13-38 (2001).
5. Moscona, A. & Moscona, H. The dissociation and aggregation of cells from organ rudiments of the early chick embryo. J Anat 86, 287-301 (1952).
6. Li, G. X., Moscona, M. H. & Moscona, A. A. Effect of embryonic age on aggregability, histogenesis and biochemical differentiation in the embryonic chick and quail neural retina. Sci Sin B 27, 371-379 (1984).
7. Williams, G. Pleiotropy, Natural Selection, and the Evolution of Senescence. Evolution 11, 398-411 (1957).
8. Kim, N. W. et al. Specific association of human telomerase activity with immortal cells and cancer. Science 266, 2011-2015 (1994).
9. Dreyer, W. J. G., W. R.; Hood, L. The Genetic, Molecular, and Cellular Basis of Antibody Formation: Some Facts and a Unifying Hypothesis. Cold Spring Harb Symp Quant Biol 32, 353-367 (1967).
10. Dreyer, W. J. & Roman-Dreyer, J. Cell-surface area codes: mobile-element related gene switches generate precise and heritable cell-surface displays of address molecules that are used for constructing embryos. Genetica 107, 249-259 (1999).
11. Wu, Q. & Maniatis, T. A striking organization of a large family of human neural cadherin-like cell adhesion genes. Cell 97, 779-790 (1999).
12. Thu, C. A. et al. Single-cell identity generated by combinatorial homophilic interactions between alpha, beta, and gamma protocadherins. Cell 158, 1045-1059 (2014).
13. Schreiner, D. & Weiner, J. A. Combinatorial homophilic interaction between gamma-protocadherin multimers greatly expands the molecular diversity of cell adhesion. Proc Natl Acad Sci USA 107, 14893-14898 (2010).
14. Kaneko, R. et al. Allelic gene regulation of Pcdh-alpha and Pcdh-gamma clusters involving both monoallelic and biallelic expression in single Purkinje cells. J Biol Chem 281, 30551-30560 (2006).
15. Lefebvre, J. L., Kostadinov, D., Chen, W. V., Maniatis, T. & Sanes, J. R. Protocadherins mediate dendritic self-avoidance in the mammalian nervous system. Nature 488, 517-521 (2012).
16. Halbleib, J. M. & Nelson, W. J. Cadherins in development: cell adhesion, sorting, and tissue morphogenesis. Genes & development 20, 3199-3214 (2006).
17. Thomson, J. A. et al. Embryonic stem cell lines derived from human blastocysts. Science 282, 1145-1147 (1998).
18. Clevers, H. Modeling Development and Disease with Organoids. Cell 165, 1586-1597 (2016).
19. West, M. D. et al. Use of deep neural network ensembles to identify embryonic-fetal transition markers: repression of COX7A1 in embryonic and cancer cells. Oncotarget 9, 7796-7811 (2018).
20. West, M. D. et al. The ACTCellerate initiative: large-scale combinatorial cloning of novel human embryonic stem cell derivatives. Regen Med 3, 287-308 (2008).
21. Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat Methods 10, 1213-1218 (2013).
22. Yang, J. & Corces, V. G. Insulators, long-range interactions, and genome function. Curr Opin Genet Dev 22, 86-92 (2012).
23. Sternberg, H. et al. A human embryonic stem cell-derived clonal progenitor cell line with chondrogenic potential and markers of craniofacial mesenchyme. Regen Med 7, 481-501 (2012).
24. West, M. D. et al. Clonal derivation of white and brown adipocyte progenitor cell lines from human pluripotent stem cells. Stem cell research & therapy 10, 7 (2019).
25. Toyota, M. et al. CpG island methylator phenotype in colorectal cancer. Proc Natl Acad Sci USA 96, 8681-8686 (1999).
26. Ogino, S. et al. Evaluation of markers for CpG island methylator phenotype (CIMP) in colorectal cancer by a large population-based sample. J Mol Diagn 9, 305-314 (2007).
27. Lund, E. G., Duband-Goulet, I., Oldenburg, A., Buendia, B. & Collas, P. Distinct features of lamin A-interacting chromatin domains mapped by ChIP-sequencing from sonicated or micrococcal nuclease-digested chromatin. Nucleus 6, 30-39 (2015).
28. Guelen, L. et al. Domain organization of human chromosomes revealed by mapping of nuclear lamina interactions. Nature 453, 948-951 (2008).
29. Khan, A. & Zhang, X. dbSUPER: a database of super-enhancers in mouse and human genome. Nucleic Acids Res 44, D164-171 (2016).
30. Kim, S., Yu, N. K. & Kaang, B. K. CTCF as a multifunctional protein in genome regulation and gene expression. Exp Mol Med 47, e166 (2015).
31. Igolkina, A. A. et al. H3K4me3, H3K9ac, H3K27ac, H3K27me3 and H3K9me3 Histone Tags Suggest Distinct Regulatory Evolution of Open and Condensed Chromatin Landmarks. Cells 8 (2019).
32. Goodman, K. M. et al. Protocadherin cis-dimer architecture and recognition unit diversity. Proc Natl Acad Sci USA 114, E9829-E9837 (2017).
33. Kehayova, P., Monahan, K., Chen, W. & Maniatis, T. Regulatory elements required for the activation and repression of the protocadherin-alpha gene cluster. Proc Natl Acad Sci USA 108, 17195-17200 (2011).
34. Sternberg, H. et al. Seven diverse human embryonic stem cell-derived chondrogenic clonal embryonic progenitor cell lines display site-specific cell fates. Regen Med (2012).
35. Jakab, K. et al. Tissue engineering by self-assembly and bio-printing of living cells. Biofabrication 2, 022001 (2010).
36. Constantinescu, D., Gray, H. L., Sammak, P. J., Schatten, G. P. & Csoka, A. B. Lamin A/C expression is a marker of mouse and human embryonic stem cell differentiation. Stem Cells 24, 177-185 (2006).
37. Shimi, T. et al. The role of nuclear lamin B1 in cell proliferation and senescence. Genes & development 25, 2579-2593 (2011).
38. West, M. D. et al. Toward a unified theory of aging and regeneration. Regen Med 14, 867-886 (2019).
39. Sternberg, H., Janus, J. & West, M. D. Defining cell-matrix combination products in the era of pluripotency. Biomatter 3 (2013).
40. Almenar-Queralt, A. et al. Chromatin establishes an immature version of neuronal protocadherin selection during the naïve-to-primed conversion of pluripotent stem cells. Nat Genet 51, 1691-1701 (2019).
41. El Hajj, N., Dittrich, M. & Haaf, T. Epigenetic dysregulation of protocadherins in human disease. Semin Cell Dev Biol 69, 172-182 (2017).
42. Kaczkowski, B. et al. Transcriptome Analysis of Recurrently Deregulated Genes across Multiple Cancers Identifies New Pan-Cancer Biomarkers. Cancer research 76, 216-226 (2016).
43. K., V. R. L. Cellular Pathology (Originally Published 1859). (Classics of Medicine, U.S.A.; 1978).
44. Warburg, O. On the origin of cancer cells. Science 123, 309-314 (1956).
45. Manzo, G. Similarities Between Embryo Development and Cancer Process Suggest New Strategies for Research and Therapy of Tumors: A New Point of View. Front Cell Dev Biol 7, 20 (2019).
46. Abe, M. et al. CpG island methylator phenotype is a strong determinant of poor prognosis in neuroblastomas. Cancer research 65, 828-834 (2005).
47. Henrich, K. O. et al. Integrative Genome-Scale Analysis Identifies Epigenetic Mechanisms of Transcriptional Deregulation in Unfavorable Neuroblastomas. Cancer research 76, 5523-5537 (2016).
48. Vega-Benedetti, A. F. et al. Clustered protocadherins methylation alterations in cancer. Clin Epigenetics 11, 100 (2019).
49. West, M. D. et al. The germline/soma dichotomy: implications for aging and degenerative disease. Regen Med 11, 331-334 (2016).
50. Rober, R. A., Weber, K. & Osborn, M. Differential timing of nuclear lamin A/C expression in the various organs of the mouse embryo and the young animal: a developmental study. Development 105, 365-378 (1989).
51. Solovei, I. et al. LBR and lamin A/C sequentially tether peripheral heterochromatin and inversely regulate differentiation. Cell 152, 584-598 (2013).
52. Tardat, M. & Dejardin, J. Telomere chromatin establishment and its maintenance during mammalian development. Chromosoma 127, 3-18 (2018).
53. van Steensel, B. & Belmont, A. S. Lamina-Associated Domains: Links with Chromosome Architecture, Heterochromatin, and Gene Repression. Cell 169, 780-791 (2017).
54. Dixon, J. R. et al. Chromatin architecture reorganization during stem cell differentiation. Nature 518, 331-336 (2015).
55. McClay, J. L. et al. A methylome-wide study of aging using massively parallel sequencing of the methyl-CpG-enriched genomic fraction from blood in over 700 subjects. Hum Mol Genet 23, 1175-1185 (2014).
56. Salpea, P. et al. Postnatal development- and age-related changes in DNA-methylation patterns in the human genome. Nucleic Acids Res 40, 6477-6494 (2012).
57. Mah, K. M., Houston, D. W. & Weiner, J. A. The gamma-Protocadherin-C3 isoform inhibits canonical Wnt signalling by binding to and stabilizing Axin1 at the membrane. Sci Rep 6, 31665 (2016).
58. Ocampo, A. et al. In Vivo Amelioration of Age-Associated Hallmarks by Partial Reprogramming. Cell 167, 1719-1733 e1712 (2016).
59. Lanza, R. P., Cibelli, J. B. & West, M. D. Human therapeutic cloning. Nat Med 5, 975-977 (1999).
60. Plath, K. & Lowry, W. E. Progress in understanding reprogramming to the induced pluripotent state. Nat Rev Genet 12, 253-265 (2011).
61. Soufi, A., Donahue, G. & Zaret, K. S. Facilitators and impediments of the pluripotency reprogramming factors' initial engagement with the genome. Cell 151, 994-1004 (2012).
62. Trapnell, C. et al. Differential gene and transcript expression analysis of RNA-seq experiments with TopHat and Cufflinks. Nat Protoc 7, 562-578 (2012).
63. Corces, M. R. et al. An improved ATAC-seq protocol reduces background and enables interrogation of frozen tissues. Nat Methods 14, 959-962 (2017).
64. Bentsen, M. et al. ATAC-seq footprinting unravels kinetics of transcription factor binding during zygotic genome activation. Nat Commun 11, 4267 (2020).
65. Zhang, Y. et al. Model-based analysis of ChIP-Seq (MACS). Genome Biol 9, R137 (2008).
66. Fornes, O. et al. JASPAR 2020: update of the open-access database of transcription factor binding profiles. Nucleic Acids Res 48, D87-D92 (2020).
67. Robinson, J. T. et al. Integrative genomics viewer. Nat Biotechnol 29, 24-26 (2011).
68. Krueger, F. & Andrews, S. R. Bismark: a flexible aligner and methylation caller for Bisulfite-Seq applications. Bioinformatics 27, 1571-1572 (2011).
69. Juhling, F. et al. metilene: fast and sensitive calling of differentially methylated regions from bisulfite sequencing data. Genome Res 26, 256-262 (2016).
70. Mahmoudi, S., and Brunet, A. (2012) Aging and reprogramming: a two-way street. Curr Opin Cell Biol 24, 744-756
71. Lapasset, L., Milhavet, O., Prieur, A., Besnard, E., Babled, A., Ait-Hamou, N., Leschik, J., Pellestor, F., Ramirez, J. M., De Vos, J., Lehmann, S., and Lemaitre, J. M. (2011) Rejuvenating senescent and centenarian human cells by reprogramming through the pluripotent state. Genes Dev 25, 2248-2253
72. Ocampo, A., Reddy, P., Martinez-Redondo, P., Platero-Luengo, A., Hatanaka, F., Hishida, T., Li, M., Lam, D., Kurita, M., Beyret, E., Araoka, T., Vazquez-Ferrer, E., Donoso, D., Roman, J. L., Xu, J., Rodriguez Esteban, C., Nunez, G., Nunez Delicado, E., Campistol, J. M., Guillen, I., Guillen, P., and Izpisua Belmonte, J. C. (2016) In Vivo Amelioration of Age-Associated Hallmarks by Partial Reprogramming. Cell 167, 1719-1733 e1712
73. West, M. D., Labat, I., Sternberg, H., Larocca, D., Nasonkin, I., Chapman, K., Singh, R., Larocca, J., Makarev, E., Aliper, A. M., Kazennov, A., Alekseenko, A., Shuvalov, N., Cheskidova, E., Alekseev, A., Artemov, A., Putin, E., Mamoshina, P., Nikita P., Copeland, K., Korzinkin, M., Lezhnina, K., Izumchenko, E., Zhavoronkov, A. (2017) Use of Deep Neural Network Ensembles to Identify Embryonic-Fetal Transition Markers: Repression of COX7A1 in Embryonic and Cancer Cells. Oncotarget
74. Zhou, W., Choi, M., Margineantu, D., Margaretha, L., Hesson, J., Cavanaugh, C., Blau, C. A., Horwitz, M. S., Hockenbery, D., Ware, C., and Ruohola-Baker, H. (2012) HIF1alpha induced switch from bivalent to exclusively glycolytic metabolism during ESC-to-EpiSC/hESC transition. EMBO J 31, 2103-2116
75. Mishra, N., Timilsina, U., Ghimire, D., Dubey, R. C., and Gaur, R. (2017) Downregulation of cytochrome c oxidase subunit 7A1 expression is important in enhancing cell proliferation in adenocarcinoma cells. Biochem Biophys Res Commun 482, 713-719
76. West, M. D., Sargent, R. G., Long, J., Brown, C., Chu, J. S., Kessler, S., Derugin, N., Sampathkumar, J., Burrows, C., Vaziri, H., Williams, R., Chapman, K. B., Larocca, D., Loring, J. F., and Murai, J. (2008) The ACTCellerate initiative: large-scale combinatorial cloning of novel human embryonic stem cell derivatives. Regen Med 3, 287-308
77. West, M. D., Chang, C. F., Larocca, D., Li, J., Jiang, J., Sim, P., Labat, I., Chapman, K. B., Wong, K. E., Nicoll, J., Van Kanegan, M. J., de Grey, A., Nasonkin, I. O., Stahl, A., and Sternberg, H. (2019) Clonal derivation of white and brown adipocyte progenitor cell lines from human pluripotent stem cells. Stem Cell Res Ther 10, 7
78. Ohnuki, M., Tanabe, K., Sutou, K., Teramoto, I., Sawamura, Y., Narita, M., Nakamura, M., Tokunaga, Y., Nakamura, M., Watanabe, A., Yamanaka, S., and Takahashi, K. (2014) Dynamic regulation of human endogenous retroviruses mediates factor-induced reprogramming and differentiation potential. Proc Natl Acad Sci USA 111, 12426-12431
79. Olova, N., Simpson, D. J., Marioni, R. E., and Chandra, T. (2019) Partial reprogramming induces a steady decline in epigenetic age before loss of somatic identity. Aging Cell 18, e12877
80. Tanabe, K., Nakamura, M., Narita, M., Takahashi, K., and Yamanaka, S. (2013) Maturation, not initiation, is the major roadblock during reprogramming toward pluripotency from human fibroblasts. Proc Natl Acad Sci USA 110, 12172-12179
81. Kurita, M., Araoka, T., Hishida, T., O'Keefe, D. D., Takahashi, Y., Sakamoto, A., Sakurai, M., Suzuki, K., Wu, J., Yamamoto, M., Hernandez-Benitez, R., Ocampo, A., Reddy, P., Shokhirev, M. N., Magistretti, P., Nunez Delicado, E., Eto, H., Harii, K., and Izpisua Belmonte, J. C. (2018) In vivo reprogramming of wound-resident cells generates skin epithelial tissue. Nature 561, 243-247
82. Tang, Y., and Cheng, L. (2017) Cocktail of chemical compounds robustly promoting cell reprogramming protects liver against acute injury. Protein Cell 8, 273-283
83. Marion, R. M., and Blasco, M. A. (2017) Long Live Partial Reprogramming. Circ Res 120, 1381-1383
84. Tamanini, S., Comi, G. P., and Corti, S. (2018) In Vivo Transient and Partial Cell Reprogramming to Pluripotency as a Therapeutic Tool for Neurodegenerative Diseases. Mol Neurobiol 55, 6850-6862
85. Beyret, E., Martinez Redondo, P., Platero Luengo, A., and Izpisua Belmonte, J. C. (2018) Elixir of Life: Thwarting Aging With Regenerative Reprogramming. Circ Res 122, 128-141
86. Doeser, M. C., Scholer, H. R., and Wu, G. (2018) Reduction of Fibrosis and Scar Formation by Partial Reprogramming In Vivo. Stem Cells 36, 1216-1225

1. A method for treating cancer in a subject, a method comprising steps: 1) obtaining a biological sample from the subject comprising cancer cells; 2) identifying cancer cells that exhibit an embryonic phenotype; 3) identifying one or more isoforms of a clustered protocadherin locus expressed in the cancer cell that exhibit an embryonic phenotype; and 4) administering to the subject an anti-cancer vaccine comprising an antigen, thereby inducing an immune response.

2. The method of claim 1, wherein the identified one or more isoforms are members of the alpha cluster protocadherins and/or beta cluster protocadherins.

3. The method of claim 2, wherein the one or more isoforms are PCDHA1, PCDHA3, PCDHA6, PCDHB3, or a combination thereof.

4. (canceled)

5. The method of claim 1, wherein the anti-cancer vaccine is mRNA.

6. The method of claim 5, wherein the mRNA encodes one or more isoforms of the alpha cluster protocadherins and/or beta cluster protocadherins.

7. The method of claim 6, wherein the mRNA encodes PCDHA1, PCDHA3, PCDHA6, PCDHB3, or a combination thereof.

8.-10. (canceled)

11. The method of claim 1, wherein the anti-cancer vaccine is one or more polypeptides of isoforms of the alpha cluster protocadherins and/or beta cluster protocadherins, or fragments thereof.

12. The method of claim 11, wherein the one or more polypeptides are PCDHA1, PCDHA3, PCDHA6, PCDHB3, fragments thereof, or a combination thereof.

13.-21. (canceled)

22. A method for inducing an immune response against mammalian cancer cells in a subject, comprised of the steps: 1) obtaining a biological sample from the subject comprising cancer cells; 2) identifying cancer cells that exhibit an embryonic phenotype; 3) identifying one or more isoforms of a clustered protocadherin locus expressed in the cancer cell that exhibit an embryonic phenotype; and 4) administering to the subject genetically-modified immune cells capable of generating an immune response to the cancer in the subject.

23. The method of claim 22, wherein the identified one or more isoforms are PCDHA1, PCDHA3, PCDHA6, PCDHB3, or a combination thereof.

24. The method of claim 22, wherein the genetically-modified immune cells comprise an antigen-binding domain, wherein the antigen-binding domain binds PCDHA1, PCDHA3, PCDHA6, PCDHB3, fragments thereof, or a combination thereof.

25.-28. (canceled)

29. The method of claim 22, wherein the genetically-modified immune cells are Chimeric Antigen Receptor T-cells (CAR T-cells).

30. The method of claim 29, wherein the CAR comprises an antigen binding domain that binds an isoform of the clustered protocadherin locus expressed in the cancer cell.

31. (canceled)

32. The method of claim 30, wherein the isoform are PCDHA1, PCDHA3, PCDHA6, PCDHB3, fragment thereof, or a combination thereof.

33.-35. (canceled)

36. A method for inducing an immune response against mammalian cancer cells in a subject, comprised of the steps: 1) obtaining a biological sample from the subject comprising cancer cells; 2) identifying cancer cells that exhibit an embryonic phenotype; 3) identifying one or more isoforms of a clustered protocadherin locus expressed in the cancer cell that exhibit an embryonic phenotype; and 4) administering to the subject an immunoglobulin superfamily member to direct an immune response specifically to the cancer cells.

37. The method of claim 36, wherein the one or more isoforms identified in the cancer cells are PCDHA1, PCDHA3, PCDHA6, PCDHB3, or a combination thereof.

38. The method of claim 36, wherein the immunoglobulin superfamily member is a monoclonal or polyclonal antibody.

39. The method of claim 38, wherein the antibody binds PCDHA3 or PCDHB3.

40.-53. (canceled)

54. The method of claim 1, wherein the method further comprises administering a chemotherapeutic agent to the subject.

55. The method of claim 54, wherein the chemotherapeutic agent is a DNA damaging agent, checkpoint inhibitor, antibody, alkylating agent, antimetabolites, anthracyclines, nitrosoureas, topisomerase inhibitor, isomerase inhibitor, mitotic inhibitor, tyrosine kinase inhibitors, protease inhibitor, or a combination thereof.

56.-57. (canceled)

58. The method of claim 1, wherein the cancer is B cell cancer, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, bone cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, polycythemia vera, lymphoma, multiple myeloma, bladder cancer, colon cancer, gynecologic cancers, renal cancer, laryngeal cancer, oral cancer, head and neck cancer, or skin cancer.

59.-60. (canceled)
